(19) United States
(12) Patent Application Publication
(10) Pub. No.: US 2022/0372101 A1
(43) Pub. Date: Nov. 24, 2022

(54) NOVEL RECEPTORS HAVING A HETEROLOGOUS STOP TRANSFER SEQUENCE FOR LIGAND-DEPENDENT TRANSCRIPTIONAL REGULATION

(71) Applicant: The Regents of the University of California, Oakland, CA (US)

(72) Inventors: Kole T. ROYBAL, Oakland, CA (US); Raymond LIU, Oakland, CA (US); Iowis ZHU, Oakland, CA (US)

(21) Appl. No.: 17/763,110

(22) PCT Filed: Sep. 23, 2020
(86) PCT No.: PCT/US2020/052337

§ 371 Date: Mar. 23, 2022

Related U.S. Application Data
(60) Provisional application No. 62/905,262, filed on Sep. 24, 2019.

(51) Int. Cl. C07K 14/705; C07K 14/47; C07K 16/28; A61P 35/02; A61K 35/17; A61K 45/06; A61K 39/395; A61K 38/17; C07K 14/575; A61K 38/22; A61P 35/00

(52) U.S. Cl. CPC C07K 14/705; C07K 14/47; C07K 16/2803; A61P 35/02; A61K 35/17; A61K 45/06; A61K 39/3955; A61K 38/1709; A61K 38/1774; C07K 14/70517; C07K 14/575; A61K 38/22; A61K 38/177; A61P 35/00; A61K 2039/505

(57) ABSTRACT

The present disclosure generally relates to, among other things, a new class of receptors engineered to modulate transcription in a ligand-dependent manner. The new receptors provide a selectable degree of noise, expression level, and signal to noise ratio. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### CROSS-REFERENCE TO RELATED APPLICATIONS

This application claims priority to U.S. Provisional Patent Application No. 62/905,262, filed Sep. 24, 2019, the disclosure of which is incorporated by reference herein in its entirety, including any drawings.

### STATEMENT REGARDING FEDERALLY SPONSORED R&D

This invention was made with government support under grant no. OD025751 awarded by The National Institutes of Health. The government has certain rights in the invention.

### INCORPORATION OF THE SEQUENCE LISTING

This application contains a Sequence Listing which is hereby incorporated by reference in its entirety. The accompanying Sequence Listing text file, named “048536_657001WO_Sequence_Listing_ST25.txt,” was created on Sep. 23, 2020 and is 828 KB.

### FIELD

The present disclosure relates generally to new synthetic cellular receptors that bind cell-surface ligands and have selectable specificities and activities. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### BACKGROUND

An important problem which limits the development of engineered cell therapies in humans is the ability to regulate therapeutic gene expression and engineered cell activity. For example, the first generation of chimeric antigen receptor T cells (CAR-T) lack the ability to modulate or turn off CAR-T activity when needed; other problems include off-target activity and off-tumor/on-target activity (i.e., wherein the CAR-T target antigen is also found on normal cells outside the tumor). One possible solution to these problems is to use a synthetic receptor that is capable of modifying gene expression and/or cellular behavior.

Notch receptors are transmembrane proteins that mediate cell-cell contact signaling and play a central role in development and other aspects of cell-to-cell communication, e.g., communication between two contacting cells, in which one contacting cell is a “receiver” cell and the other contacting cell is a “sender” cell. Notch receptors expressed in a receiver cell recognize their ligands (the delta/serrate/lag, or “DSL” family of proteins) expressed on a sending cell. The engagement of notch and delta on these contacting cells leads to a two-step proteolysis of the notch receptor, which ultimately causes the release of the intracellular portion of the receptor (“ICD”) from the membrane into the cytoplasm. Notch has a matrix metalloprotease cleavage site (denoted “S2”), which, when the receptor is not activated is protected from cleavage by the Notch negative regulatory region (“NRR”). The NRR consists of three LIN-12-Notch repeat (“LNR”) modules and a heterodimerization domain (“HD”). It is believed that this proteolysis is regulated by the force exerted by the sending cell: the DSL ligand pulls on the Notch receptor, which changes the conformation of the NRR and exposes the metalloprotease site. This is cleaved by a constitutively active protease (such as ADAM10), which releases the extracellular binding portion and negative regulatory region of the receptor. Release of the ligand binding portion of the receptor in turn exposes another cleavage site (denoted “S3”), which is cleaved by γ-secretase within the cell membrane: this cleavage releases the nuclear homing ICD from the cell membrane. W.R. Gordon et al., Dev Cell (2015) 33:729-36. This released domain alters receiver cell behavior by regulating transcription. Notch receptors are involved in and are required for a variety of cellular functions during development, and are important for the function of a vast number of cell-types across species.

Examples of existing first-generation synthetic derivatives of Notch receptors, which are often referred to as “SynNotch,” employ this straightforward signaling behavior by replacing the extracellular ligand-binding domain, which in wild-type Notch contains multiple EGF-like repeats, with an antibody derivative, and replacing the cytoplasmic domain with a transcription activator of choice, but still relying on the Notch NRR (L. Morsut et al., Cell (2016) 164:780-91) and the standard two-step proteolysis described above. Additionally, the NRR spans approximately 160 amino acids, making this domain alone about three times the size of some mature proteins, such as insulin or epidermal growth factor (EGF). This makes expression of the receptor less efficient, and can exceed the capacity of some widely used cloning and transfection vectors.

Receptors, whether native or synthetic, have varying characteristics, such as noise (i.e., the level of expression induced in the absence of the intended ligand), and signal or sensitivity (the amount of expression induced by binding of the intended ligand). Generally, Notch and SynNotch signaling correlates with ligand binding, but it is difficult to adjust the sensitivity and response of the receptor. More tools are needed in order to provide synthetic receptors with a wide range of characteristics.

### SUMMARY

The present disclosure describes a previously unknown feature of Notch and SynNotch receptors, the stop-transfer sequence (STS). The STS comprises a charged, lipophobic sequence. Without being bound by any theory, the STS serves as a membrane anchor, and is believed to prevent passage of the intracellular domain into the plasma membrane. Surprisingly, altering this sequence dramatically affects the signal characteristics, such as the noise and expression/signal levels, of the receptor. The disclosure provides synthetic chimeric receptors that exhibit a range of signal characteristics determined by the STS. These receptors provide a range of sensitivity, including a receptor that is sensitive to the degree of T cell activation when it is expressed in a T cell. In such receptors, when expressed in a T cell, a higher ligand-induced signal is obtained when the T cell is activated, as compared to the ligand-induced signal when the T cell is not activated.

The present disclosure provides, inter alia, novel receptors containing a heterologous STS. Since this feature prevents the receptor from being pulled through the plasma membrane during ligand binding, it is believed that the modulation of the STS facilitates the optimization and/or improvement of the expression and activity of the chimeric receptors disclosed herein, which in turn can be particularly useful in modulating cell activity and/or in treating health conditions (e.g., diseases).

In one aspect, provided herein is a chimeric receptor polypeptide comprising, from N-terminus to C-terminus: (a) an extracellular ligand-binding domain having a binding affinity for a selected ligand; (b) a linking polypeptide having: (i) at least about 80% sequence identity to a Notch juxtamembrane domain (JMD); (ii) at least about 80% sequence identity to a Notch JMD in which the LIN-12-Notch repeat (LNR) and/or a heterodimerization domain (HD) of a Notch receptor has been deleted; (iii) at least about 80% sequence identity to a polypeptide hinge domain; (iv) at least about 80% sequence identity to a Robo1 JMD including at least one fibronectin repeat; or (v) a polypeptide having about 2 to about 40 amino acids; (c) a transmembrane domain (TMD) comprising one or more ligand-inducible proteolytic cleavage sites; (d) an STS, wherein the STS is heterologous to the TMD; and (e) an intracellular domain (ICD) comprising a transcriptional regulator, wherein binding of the selected ligand to the extracellular ligand-binding domain induces cleavage at the ligand-inducible proteolytic cleavage site between the transcriptional regulator and the linking polypeptide.

Non-limiting exemplary embodiments of the chimeric polypeptides provided herein include chimeric polypeptides wherein the STS is selected from the group of STS sequences consisting of APLP1, APLP2, APP, TGBR3, CSF1R, CXCL16, CX3CL1, DAG1, DCC, DNER, DSG2, CDH1, GHR, HLA-A, IFNAR2, IGF1R, IL1R1, ERN2, KCNE1, KCNE2, CHL1, LRP1, LRP2, LRP18, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, KL, IL6R, EFNB1, CD44, CLSTN1, LRP8, PCDHGC3, NRG1, LRP1B, JAG2, EFNB2, DLL1, CLSTN2, EPCAM, ErbB4, KCNE3, CDH2, NRG2, PTPRK, BTC, EPHA4, IL1R2, KCNE4, SCN2B, Nradd, PTPRM, Notch1, Notch2, Notch3, and Notch4.

In some embodiments, the extracellular domain includes an antigen-binding moiety capable of binding to a ligand on the surface of a cell. In some embodiments, the cell is a pathogen. In some embodiments, the ligand includes a protein or a carbohydrate. In some embodiments, the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, BCMA (CD269), ALPPL2, GFP, eGFP, and SIRPα.

In another aspect, provided herein are nucleic acids comprising a nucleotide sequence encoding a chimeric polypeptide as disclosed herein. In some embodiments, the nucleotide sequence is incorporated into an expression cassette or an expression vector.

In another aspect, provided herein are recombinant cells including (a) a chimeric polypeptide as disclosed herein and/or (b) a recombinant nucleic acid as disclosed herein. In another aspect, further provided herein are cell cultures including at least one recombinant cell as disclosed herein and a culture medium.

In another aspect, provided herein are pharmaceutical compositions including a pharmaceutically acceptable carrier and one or more of the following: (a) a recombinant nucleic acid as disclosed herein, and (b) a recombinant cell as disclosed herein. In some embodiments, the disclosed pharmaceutical composition includes a recombinant nucleic acid as disclosed herein and a pharmaceutically acceptable carrier. In some embodiments, the recombinant nucleic acid is encapsulated in a viral capsid or a lipid nanoparticle.

In another aspect, provided herein are methods for modulating an activity of a cell, including: (a) providing a recombinant cell of the disclosure, and (b) contacting it with a selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell. Another aspect relates to methods for inhibiting an activity of a target cell in an individual, including administering to the individual an effective number of the recombinant cell of the disclosure, wherein the recombinant cell inhibits an activity of the target cell in the individual.

In another aspect, provided herein are methods for treating a health condition (e.g., disease) in an individual, the methods comprising a step of administering to the individual an effective number of the recombinant cell of the disclosure, wherein the recombinant cell treats the health condition in the individual.

In another aspect, provided herein are systems for modulating an activity of a cell, inhibiting a target cancer cell, or treating a health condition (e.g., disease) in an individual in need thereof, wherein the system includes one or more of: a chimeric polypeptide of the disclosure; a polynucleotide of the disclosure; a recombinant cell of the disclosure; or a pharmaceutical composition of the disclosure.

In another aspect, provided herein are methods for making a recombinant cell of the disclosure, including: (a) providing a cell capable of protein expression; and (b) contacting the provided cell with a recombinant nucleic acid of the disclosure. In some embodiments, the cell is obtained by leukapheresis performed on a sample obtained from a human subject or patient, and the cell is contacted ex vivo. In some embodiments, the recombinant nucleic acid is encapsulated in a viral capsid or a lipid nanoparticle.

In another aspect, provided herein is the use of one or more of: a chimeric polypeptide of the disclosure, a polynucleotide of the disclosure, a recombinant cell of the disclosure, or a pharmaceutical composition of the disclosure, for the treatment of a health condition (e.g., disease). In some embodiments, the health condition is cancer.

In another aspect, provided herein is the use of one or more of: a chimeric polypeptide of the disclosure, a polynucleotide of the disclosure, a recombinant cell of the disclosure, or a pharmaceutical composition of the disclosure, in the manufacture of a medicament for the treatment of a health condition (e.g., disease).

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 schematically illustrates differences between a SynNotch receptor, an Fn Notch receptor, a Mini Notch receptor, a Minimal linker Notch receptor, and a Hinge Notch receptor. The SynNotch receptor differs from the wild-type Notch receptor in that the large extracellular ligand-binding region comprising EGF repeats is replaced with a heterologous extracellular ligand-binding domain, in this example, with an scFv specific for the CD19 B-cell antigen. In the exemplary Fn Notch receptor, the Notch regulatory region and juxtamembrane domain has been deleted and replaced with a truncated Robo1 juxtamembrane domain that includes three fibronectin repeats (Fn). In the exemplary Mini Notch receptor, the Notch negative regulatory region (NRR) that is present in SynNotch is deleted. In the exemplary Minimal Linker Notch receptor, all of the extracellular Notch sequence is deleted and replaced with a linking polypeptide, such as a (GGS)ₙ linker. In the exemplary Hinge Notch receptor, the extracellular Notch sequence is deleted and replaced with a linking polypeptide domain that promotes oligomerization, such as dimerization or trimerization, in this example based on the hinge region of CD8α.

FIGS. 2A-2B depict a schematic synNotch receptor and reporter system as used in Example 5. In an exemplary experiment, as shown in FIG. 2A, T cells express the receptor and carry a reporter plasmid containing transcription factor-specific response elements (e.g., UAS response elements recognized by Gal4-VP64 transcriptional activators) driving a fluorescent reporter protein (e.g., BFP). SynNotch receptor activation is measured by recording the BFP reporter signal in T cells after co-incubation with cell expressing a target surface antigen (e.g., CD19). An example of a “successful” synNotch activation is shown as a flow cytometry histogram, with BFP reporter signal on the x-axis and counts on the y-axis. A representative synNotch receptor activation plot is depicted, where a CD19-presenting cell (for example, a K562 CD19+ cell) induces BFP expression and signal greater than control cells (e.g., K562 without CD19). Screening is performed with synNotch receptors containing STS sequences from a library of Type 1 receptor proteins reported to be regulated by gamma (γ) secretase activity. FIG. 2B schematically depicts the array of receptor proteins tested in Example 5, with the protein that was the source of the STS indicated. “Reporter” was a control T cell, a T cell carrying the reporter plasmid, but without a receptor of the disclosure.

FIG. 3 shows a heat map array of receptor protein expressions in Jurkat T cells as tested in Example 5 (as measured by myc tag expression), with the STS protein source indicated. This demonstrated that the choice of STS sequence affects the expression level of the receptor.

FIG. 4 shows an array of flow cytometry overlay plot diagrams, depicting BFP signals from Jurkat T cells expressing synNotch receptors when incubated with CD19− or CD19+ K562 target cells. The STS sources in the array are as shown in FIG. 2B and FIG. 3.

FIG. 5 shows a heat map of percent positive Jurkat T cells expressing the BFP reporter, subtracted from background levels of activation and normalized for myc-tag expression, as described in Example 5.

FIGS. 6A-6C schematically depicts validation of STS screen hits in the Hinge-Notch scaffold. FIG. 6A shows a diagram for a truncated Hinge Notch receptor having a heterologous STS. The STS is located immediately C-terminal to the TMD. A library of truncated Hinge Notch receptors was constructed, each having one of ten different STSs. The STS source proteins used were, in order of decreasing activation level: EPCAM (Uniprot ID P16422), LRP18 (ID Q9ZR2), JAG2 (ID Q9Y219), PTPRK (ID Q15262), NOTCH2 (ID Q04721), NRG2 (ID 014511), PTPRM (ID P28827), CXCL16 (ID Q9H2A7), NRG1 (ID Q02297), and GHR (ID P10912). In FIG. 6B, primary human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive mCitrine gene located on the reporter plasmid. Double positive cells were sorted for myc tag and mCitrine expression on Day 5 post initial T cell stimulation, and expanded further for activation testing. 1×10⁵ double positive T cells expressing anti-CD19 scFv were co-cultured with: no additions (upper trace), 1×10⁵ K562 cells (middle trace), or 1×10⁵ CD19+ K562 cells (lower trace) for 24 hours. Transcriptional activation of an inducible BFP reporter gene was subsequently measured using a Fortessa X-50 (BD Biosciences). FIG. 6C shows overlaid activation histograms of STS hits with the Hinge Notch receptor.

FIGS. 7A-7C schematically depict validation of STS screen hits in the Mini Notch scaffold. Mini Notch receptors constructed with heterologous STS. The STS is found immediately C-terminal to the TMD. Here, as shown in FIG. 7A, the heterologous STS is defined as a region of basic residues (R, K, H) that terminates before two consecutive non-basic residues. The STS source proteins used were, in order of decreasing activation level: EPCAM, PTPRK, NOTCH2, PTPRM, LRP1B, GHR, JAG2, NRG2, CXCL16, and NRG1. In FIG. 7B, primary human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive mCitrine gene located on the reporter plasmid. Double positive cells were sorted for on Day 5 post initial T cell stimulation and expanded further for activation testing. 1×10⁵ double positive T cells expressing anti-CD19 receptors are co-cultured with: no additions (upper trace), 1×10⁵ K562 cells (middle trace), or 1×10⁵ CD19+ K562 cells (lower trace) for 24 hours. Transcriptional activation of an inducible BFP reporter gene was measured using a Fortessa X-50 (BD Biosciences). The flow histograms for each receptor are shown in FIG. 7B. FIG. 7C shows the overlaid histograms for all of the constructs.

FIG. 8 shows the results of experiments comparing the Notch1 STS with the Notch2 STS. Receptors were constructed with SynNotch (using Notchl), Mini Notch1, and Hinge Notch, each with either the Notch1 STS or the Notch2 STS. Primary human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive mCitrine gene located on the reporter plasmid. Double positive cells were sorted for on Day 5 post initial T cell stimulation and expanded further for activation testing. 1×10⁵ double positive T cells expressing anti-CD19 receptors are co-cultured with: no additions (upper trace), 1×10⁵ K562 cells (middle trace), or 1×10⁵ CD19+ K562 cells (lower trace) for 24 hours. Transcriptional activation of an inducible BFP reporter gene was subsequently measured using a Fortessa X-50 (BD Biosciences). The resulting histograms are shown in the left panels of FIG. 8. The right panel of FIG. 8 shows the same data as a bar graph.

FIG. 9 schematically summarizes the results from experiments performed to test Hinge-Notch variants with different binding domains and their dependence on proteolytic activity. Primary human CD4+ T-cells were activated with anti-CD3/anti-CD28 Dynabeads and transduced with two lentiviral constructs, one expressing a hinge receptor with indicated binding head truncation variant receptor, and the other a transcriptional reporter. Cells containing both constructs were sorted on Day 5 post initial T-cell stimulation and expanded further for activation testing. For testing, 1×10⁵ double positive T-cells expressing receptors were co-cultured with 1×10⁵ K562 cells (top trace), 1×10⁵ Ligand+K562 cells (second trace from top), 1×10⁵ Ligand+K562 cells with an ADAM10 inhibitor (third trace from top), or 1×10⁵ Ligand+K562 cells with a gamma-secretase inhibitor, DAPT (bottom trace). Transcriptional activation of an inducible BFP reporter gene was subsequently measured using a Fortessa X-50.

FIG. 10 schematically summarizes the results from experiments performed to compare activation of Hinge-Notch variants with different promoters and STS domains. For testing, 1×10⁵ double positive T-cells expressing anti-CD19 receptors were co-cultured with no additions (top trace), 1×10⁵ ALPPL2+ K562 cells (second trace from top), 1×10⁵ CD19+ K562 cells (third trace from top), or 1×10⁵ ALPPL2+ CD19+ K562 cells (bottom trace). Transcriptional activation of an inducible BFP reporter gene was subsequently measured using a Fortessa X-50. Activation using murine and human original synNotch constructs were included for comparison.

FIG. 11 schematically summarizes the results from experiments for mutational analysis for the transmembrane domain (TMD) and the STS domain in Hinge-Notch constructs. Four types of exemplary Hinge Notch receptors were used in this Example, all of which including an anti-CD19 scFv domain, a truncated CD8 Hinge domain, and a Gal4VP64 domain, plus different TMD domains (CLSTN1 TMD or CLSTN2 TMD) and different STS domains (CLSTN1 STS, CLSTN2 STS, or Notch1 STS). Primary human CD4+ T-cells were activated with anti-CD3/anti-CD28 Dynabeads (Gibco) and transduced with two lentiviral constructs, one expressing a hinge receptor with TMD/STS combination as indicated, and the other a transcriptional reporter with constitutively expressed anti-ALPPL2 CAR. Cells containing both constructs were sorted for on Day 5 post initial T-cell stimulation and expanded further for activation testing. For testing, 1×10⁵ double positive T-cells expressing receptors were co-cultured with: 1×10⁵ K562 cells (“−CAR” panels, blue), or 1×10⁵ CD19+K562 cells (“−CAR” panels, red). Similarly, 1×10⁵ double positive T-cells expressing receptors were tested in the presence of CAR activity by co-culture with 1×10⁵ ALPPL2+ K562 cells (“+CAR” panels, blue), or 1×10⁵ ALPPL2+ CD19+ K562 cells (“+CAR” panels, red). Transcriptional activation of an inducible BFP reporter gene was subsequently measured using a Fortessa X-50 (BD Biosciences).

FIGS. 12A-12 schematically summarize the results from experiments for tunable, ligand-dependent expansion of T cells using Hinge-Notch-controlled expression of an engineered cytokine. FIG. 12A shows a diagram of T cells engineered with Hinge-Notch STS variants to provide ligand-triggered secretion of an engineered cytokine for autocrine and paracrine expansion of T cells. Expression profile of anti-CD19 Hinge-Notch receptors with the indicated STS modifications are shown in FIG. 12B. Primary human T-cells were activated with anti-CD3/anti-CD28 Dynabeads and transduced with two lentiviral constructs, one expressing a CAR against the MCAM antigen, and one expressing a Hinge-Notch receptor with inducible super-IL2 under Gal4-UAS control. Cells containing both constructs were sorted on Day 5 post initial T-cell stimulation and expanded further for activation testing. Receptor expression was determined by anti-myc-tag staining (y-axis).

FIG. 13 schematically summarizes the results from experiments performed to demonstrate that ligand-triggered expression of super-IL2 improves cell viability of CAR-T cells. 1×10⁵ double positive T-cells expressing anti-CD19 HingeNotch Notch1 STS receptors were co-cultured in media without IL-2, with no K562 cells (top left), with CD19+ K562 cells to trigger Hinge-Notch (top right), with MCAM+ K562 cells to trigger CAR activation (bottom left) or with MCAM+ and CD19+ K562 cells to trigger activation of both receptors (bottom right). After 9 days the proportion of live T cells by forward and side-scatter measurements using a Fortessa X-50 was assessed.

FIG. 14 schematically summarizes the results from experiments performed to demonstrate tunable proliferation of T cells with STS-variants of Hinge-Notch. Primary human T-cells were activated with anti-CD3/anti-CD28 Dynabeads and transduced with two lentiviral constructs, one expressing a CAR against the MCAM antigen, and one expressing a Hinge-Notch receptor with inducible super-IL2 under Gal4-UAS control (the right four panels). Hinge-Notch receptors containing 3 different STS variants (NRG1, Notch1, Notch2) were tested against a no Hinge-Notch control. Similarly, primary human T-cells were generated without CAR expression (left panels). T cells were stained with CellTrace Violet according to manufacturer's protocols, co-incubated with CD19+ K562 target cells in media without IL-2 and measured using a Fortessa X-50 at the indicated timepoints to assess proliferation by CTV signal decay.

FIGS. 15A-15B schematically summarize the results from experiments performed to demonstrate tunable secretion of super-IL2 with STS-variants of Hinge-Notch. Primary human T-cells were activated with anti-CD3/anti-CD28 Dynabeads and transduced with a lentiviral construct Hinge-Notch receptor with inducible super-IL2 under Gal4-UAS control

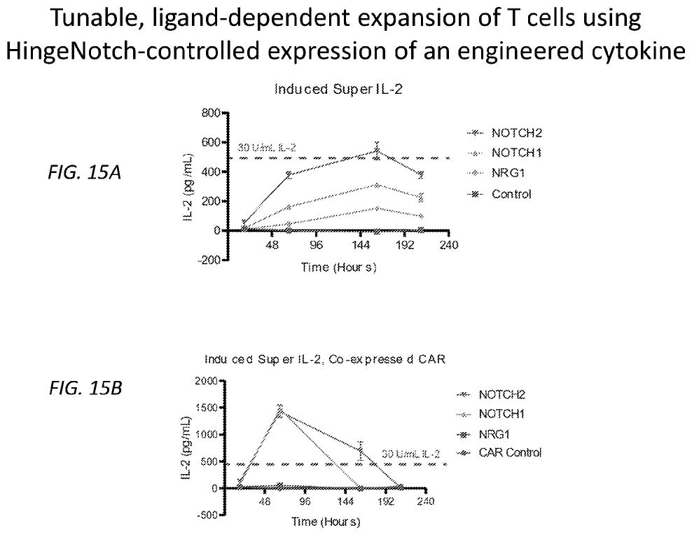

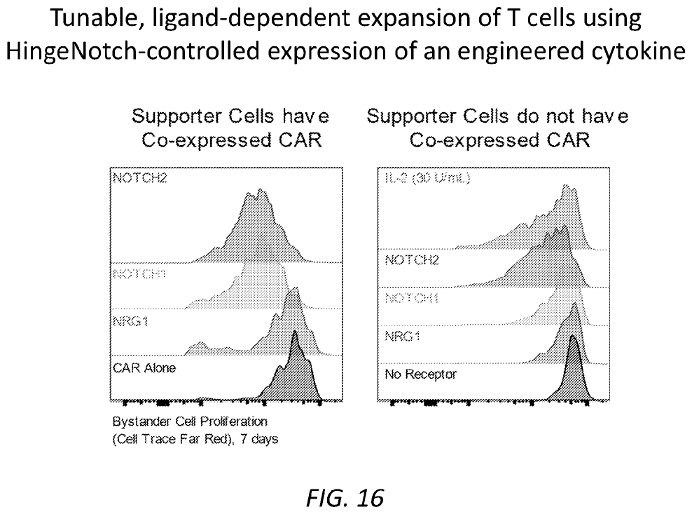

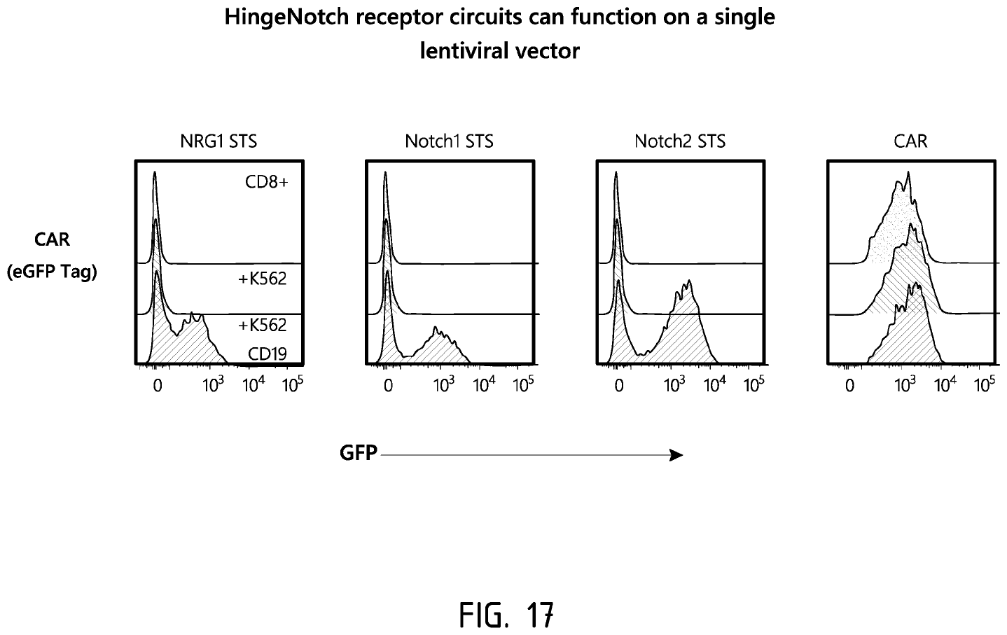

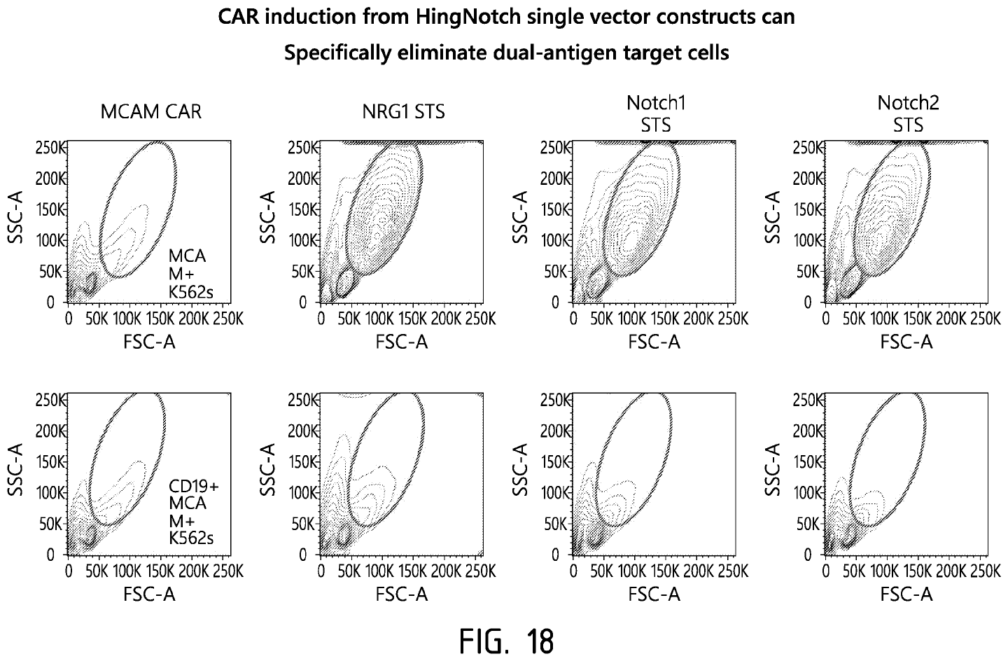

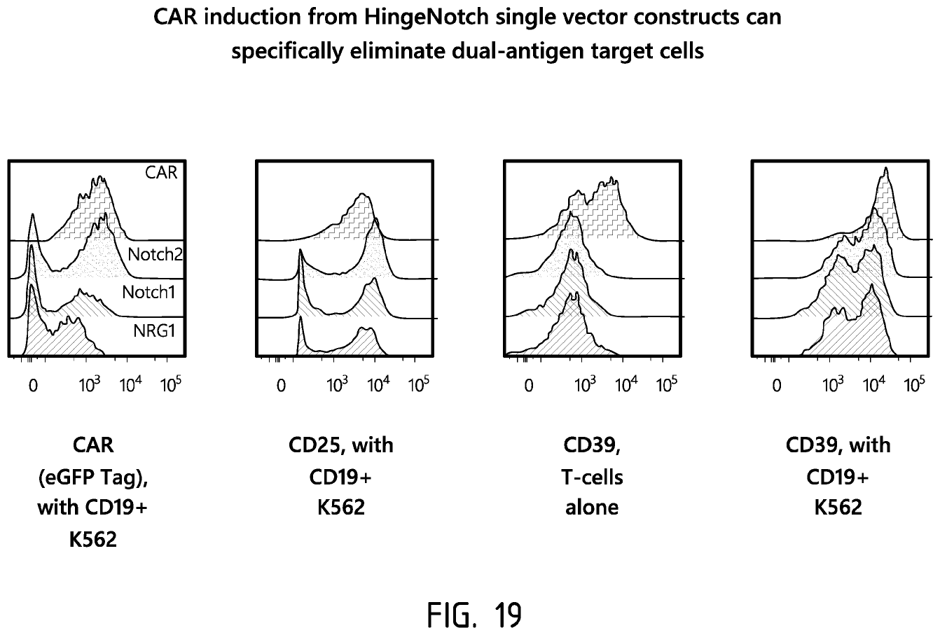

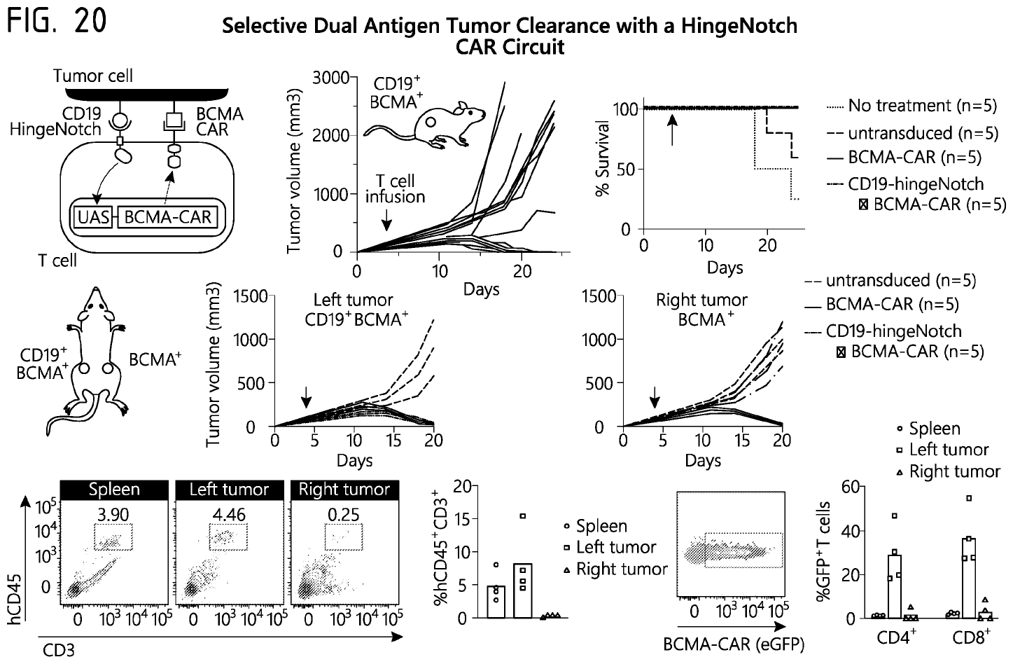

(FIG. 15A). Hinge-Notch receptors containing 3 different STS variants (NRG1, Notch1, Notch2) were tested against a no HingeNotch control. T cells were co-incubated with MCAM+ CD19+ K562 cells in media lacking IL-2, and at the indicated timepoints, supernatant IL-2 was measured using the Instant ELISA Kit according to manufacturer's protocols with a microplate reader. Red dotted line indicates a standard concentration of IL-2 used for culturing T cells. Graded secretion of super-IL2 was achieved by activation of STS-tuned Hinge-Notch receptors. For FIG. 15B, primary human T-cells were generated with an additional lentiviral vector expressing a CAR against MCAM.

FIG. 16 schematically summarizes the results from experiments performed to demonstrate tunable secretion of super-IL2 with STS-variants of Hinge-Notch enhances proliferation of bystander T cells. Primary human T-cells were activated with anti-CD3/anti-CD28 Dynabeads and transduced with a lentiviral construct comprising a Hinge-Notch receptor with inducible super-IL2 under Gal4-UAS control (right panels). HingeNotch receptors containing 3 different STS variants (NRG1, Notch1, Notch2) were tested against a no HingeNotch control. HingeNotch T cells were co-incubated with “bystander” T cells stained with CellTrace Far Red expressing a CAR against MCAM (left panel) or with no CAR (right panel). T cells were co-incubated with MCAM+ CD19+ K562 cells in media lacking IL-2, and proliferation of the bystander T cells were assessed by measuring signal decay on a Fortessa X-50.

FIG. 17 schematically summarizes the results from experiments performed to test single lentiviral vector constructs containing Hinge-Notch receptors CAR circuits. Primary human T-cells were activated with anti-CD3/anti-CD28 Dynabeads and transduced with a single lentiviral construct containing constitutively expressed Hinge-Notch receptors with an inducible anti-MCAM CAR cassette under Gal4-UAS control. Cells were sorted for Hinge-Notch receptor expression via myc-tag on Day 5 post initial T-cell stimulation and expanded further for activation testing. Three STS-variants were tested as indicated, with constitutively expressed CAR used as a control. For testing, 1×10⁵ T cells expressing anti-CD19 receptors were co-cultured with: no additions (upper trace), 5×10⁵ K562 cells (middle trace), or 5×10⁴ CD19+ K562 cells (lower trace). Transcriptional activation of the inducible CAR was subsequently measured by a GFP tag using a Fortessa X-50.

FIG. 18 schematically summarizes the results from experiments performed to demonstrate specific dual antigen target cell killing by T cells engineered with a single lentivector containing a HingeNotch CAR circuit. Primary human T-cells were activated with anti-CD3/anti-CD28 Dynabeads and transduced with a single lentiviral construct containing constitutively expressed HingeNotch -receptors with an inducible anti-MCAM CAR cassette under Gal4-UAS control. Cells were sorted for Hinge-Notch receptor expression via myc-tag on Day 5 post initial T-cell stimulation and expanded further for activation testing. Three STS-variants were tested as indicated, with constitutively expressed CAR used as a control. For testing, 1×10⁵ T-cells expressing anti-CD19 receptors were co-cultured with 5×10⁵ MCAM+ K562 cells or 5×10⁴ MCAM+ CD19+ K562 cells. Target cell killing was assessed by forward/side-scatter of the K562 population using a Fortessa X-50.

FIG. 19 schematically summarizes the results from experiments performed for testing single lentiviral vector constructs containing Hinge-Notch receptors for control of T cell activation and exhaustion. Primary human T-cells were activated with anti-CD3/anti-CD28 Dynabeads and transduced with a single lentiviral construct containing constitutively expressed Hinge-Notch receptors with an inducible anti-MCAM CAR cassette under Gal4-UAS control. Cells were sorted for Hinge-Notch receptor expression via myc-tag on Day 5 post initial T-cell stimulation and expanded further for activation testing. Three STS-variants were tested as indicated, with constitutively expressed CAR used as a control. For testing, 1×10⁵ T-cells expressing anti-CD19 receptors were co-cultured with 5×10⁴ CD19+ K562 cells. Transcriptional activation of the inducible CAR was subsequently measured by a GFP tag using a Fortessa X-50 (the left most panel). T cell activation and exhaustion were measure by expression of CD25 (the second panel from the left side) and CD39 (the third and fourth panels from the left side), respectively.

FIG. 20 schematically summarizes the results from experiments performed for in vivo testing of Hinge-Notch-to-CAR circuits. For unilateral tumors, NOD.Cg-Prkdc^{scid}Il2rg^{tm1Wjl}/SzJ (NSG) mice were implanted with 1×10⁶ K562-BCMA/CD19 tumor cells subcutaneously on the left flank. For contralateral tumors, NSG mice were implanted with 1×10⁶ K562-BCMA/CD19 tumor cells on the left flank and with 1×10⁶ K562-CD19 tumor cells on the right flank. Four days after tumor implantation, 2.5×106 engineered primary human CD4+ and CD8+ T cells (total of 5×10⁶ T cells) were infused i.v. through tail vein injection. Tumor size was monitored via caliper 2-3 per week and mice were determined to have reached endpoint when tumors measured ≥20 mm. For immunophenotypic analysis, tumors and spleens were harvested 10 days post T cell implantation. Tumors were manually minced and digested in RPMI-1640 with 4 mg/ml Collagenase IV and 0.1 mg/ml DNase I at 37° C. for 30 min and spleens were manually dissociated and subjected to red blood cell lysis. The following antibodies were used: anti- CD45, anti-CD3, anti-CD4, and anti-CD8. Dead cells were excluded with Draq7. Samples were analyzed using FACSymphony X50 SORP and data was analyzed using FlowJo software.

### DETAILED DESCRIPTION OF THE DISCLOSURE

The present disclosure generally relates to, among other things, a new class of engineered chimeric polypeptide receptors which modulate transcriptional regulation in a ligand-dependent manner. These new receptors have heterologous stop-transfer sequences, which permit the construction of receptors having a broad range of desired characteristics. For example, by altering the STS as described herein, and selecting different juxtamembrane domains, one can produce receptors having different noise levels (i.e., the basal level of receptor activation in the absence of the selected target ligand), different signal levels (i.e., the expression level obtained in the presence of the selected target ligand), and different signal:noise ratios. This principle is applicable to a variety of Notch-like Type 1 receptors, including SynNotch, as further described herein.

The general structure of the synthetic receptors of the disclosure is a single-span transmembrane protein (i.e., Type 1) having a TMD that has a cleavage site for γ-secretase, comprising from N-terminus to C-terminus (1) an extracellular ligand-binding domain; (2) a linking polypeptide joining the extracellular ligand-binding domain and the TMD; (3) a transmembrane domain comprising one or more protease cleavage sites; (4) an STS; and (5) an intracellular domain that includes a transcriptional regulator.

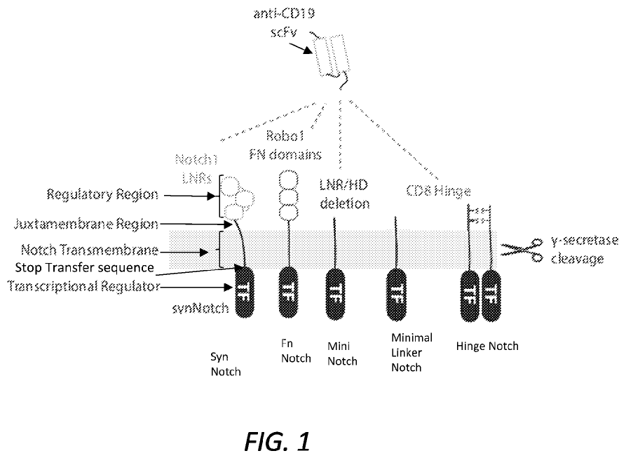

Several distinct classes of synthetic receptor are discussed and/or disclosed herein. As depicted schematically in FIG. 1, “SynNotch” receptors comprise a heterologous extracellular ligand binding domain, a linking polypeptide having substantial sequence identity with a Notch receptor including the NRR, a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor (lacking the NRR), a TMD, and an ICD. “Minimal Linker Notch” receptors comprise a heterologous extracellular ligand binding domain, a linking polypeptide lacking substantial sequence identity with a Notch receptor (e.g., a synthetic (GGS)ₙ polypeptide sequence), a TMD, and an ICD. “Hinge Notch” receptors comprise a heterologous extracellular ligand binding domain, a hinge sequence comprising an oligomerization domain (i.e., a domain that promotes dimerization, trimerization, or higher order multimerization with a synthetic receptor and/or an existing host receptor), a TMD, and an ICD. Surprisingly, the MiniNotch, Fn Notch, Hinge Notch, and Minimal Linker Notch receptors, even though derived from Notch, do not require the Notch regulatory regions previously thought to be essential for the activity of the receptors. All of these receptor classes are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Although various features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In addition, the materials, methods, and examples are illustrative only and not intended to be limiting.

### DEFINITIONS

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration

The term “heterologous” refers to a polypeptide sequence or domain which is not native to a flanking sequence, e.g., wherein the heterologous sequence is not found in nature coupled to the polypeptide sequences occurring at one or both ends. Thus, for example, a Notch4 STS is heterologous to a Notch1 TMD.

The terms “host cell” and “recombinant cell” are used interchangeably herein. It is understood that such terms, as well as “cell”, “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the originally cell or cell line.

The term “operably linked”,” as used herein, denotes a physical or functional linkage between two or more elements, e.g., polypeptide sequences or polynucleotide sequences, which permits them to operate in their intended fashion.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof

As used herein, and unless otherwise specified, a “therapeutically effective amount” of an agent is an amount sufficient to provide a therapeutic benefit in the treatment or management of a health condition, such as a disease (e.g., a cancer), or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” is an individual under the care of a physician. Thus, the subject can be a human individual or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

One skilled in the art will understand that the chimeric receptors disclosed herein provide signals having a range of characteristics, from low to high ligand-induced transduction and (independently) low to moderate non-induced signal transduction. This range of activities is a new feature that can be exploited to enhance and tune the actions of engineered cells. Further, as described in greater detail below, a number of the receptor variants disclosed herein exhibit improved expression compared to existing SynNotch receptors.

### NOTCH RECEPTORS

Notch receptors are large transmembrane proteins that normally communicate signals upon binding to surface-bound ligands expressed on adjacent cells. Notch signals rely on cell-cell contact. Evolutionary divergence of vertebrates and invertebrates was accompanied by at least two rounds of gene duplication in the Notch lineage: flies possess a single Notch gene, worms two (GLP-1 and LIN-12), and mammals four (NOTCH1-4). Transduction of Notch signals relies on three key events: (i) ligand recognition; (ii) conformational exposure of the ligand-dependent cleavage site, and (iii) assembly of nuclear transcriptional activation complexes.

Canonical Notch signals are transduced by a process called regulated intramembrane proteolysis. Notch receptors are normally maintained in a resting, proteolytically resistant conformation on the cell surface, but ligand binding initiates a proteolytic cascade that releases the intracellular domain of the receptor (ICD) from the membrane. The critical, regulated cleavage step is effected by ADAM metalloproteases and occurs at a site called S2 immediately external to the plasma membrane. This truncated receptor, dubbed NEXT (for Notch extracellular truncation), remains membrane-tethered until it is processed at site S3 by γ-secretase, a multiprotein enzyme complex.

After γ-secretase cleavage, the ICD ultimately enters the nucleus, where it nucleates assembly of a transcriptional activation complex that contains a DNA-binding transcription factor, and a transcriptional coactivator of the Mastermind family. This complex then engages one or more additional coactivator proteins such as p300 to recruit the basal transcription machinery and activate the expression of downstream target genes.

Notch receptors have a modular domain organization. The ectodomains of Notch receptors consist of a series of N-terminal epidermal growth factor (EGF)-like repeats that are responsible for ligand binding. O-linked glycosylation of these EGF repeats, including modification by O-fucose, Fringe, and Rumi glycosyltransferases, also modulates the activity of Notch receptors in response to different ligand subtypes in flies and mammals.

The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

### COMPOSITIONS OF THE DISCLOSURE

The receptors of the disclosure provide a range of sensitivity, including a receptor that, when expressed in a T cell, exhibits a higher ligand-induced signal when the T cell is activated. Additionally, by omitting the Notch/SynNotch regulatory regions, some polynucleotides encoding the receptors of the disclosure can be made smaller than SynNotch-encoding polynucleotides, which enables the use of vectors having more limited capacity, or the inclusion of additional elements that would otherwise be excluded due to vector capacity-related size constraints.

As described in greater detail below, several chimeric polypeptide receptors disclosed herein have improved signal transduction compared to existing SynNotch receptors and provide a more modular platform for engineering. Improved signal transduction can take the form of higher stimulated (ligand-induced) transduction, reduced baseline (non- stimulated) transduction, and higher ratios of stimulated to baseline transduction. Signal transduction can be measured based on the expression of a reporter gene or other signal. Existing SynNotch receptors can be engineered with ligand-binding domains such scFvs and nanobodies, but it has been difficult to use natural extracellular domains from other receptors or ligands on SynNotch receptors. In contrast, the second-generation Notch receptors disclosed herein work well with extracellular ligand binding domains comprising non-antibody derived ligand binding domains, thus expanding the landscape of targetable diseases and tissues.

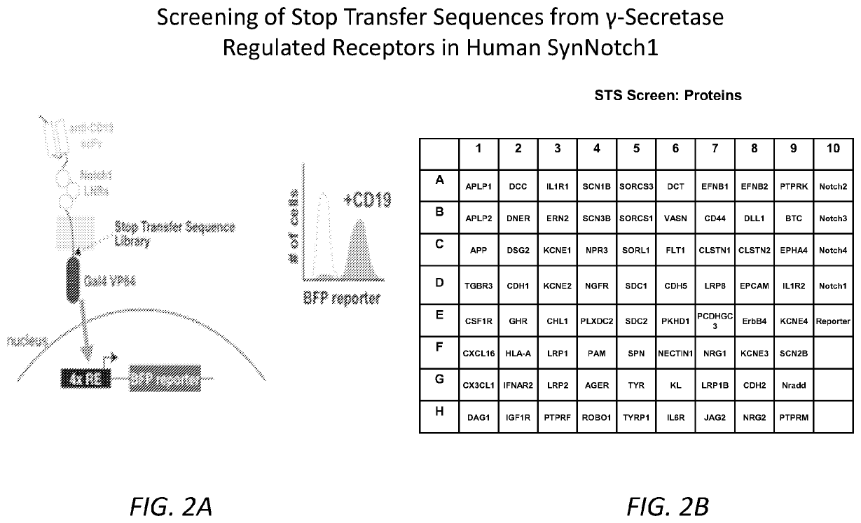

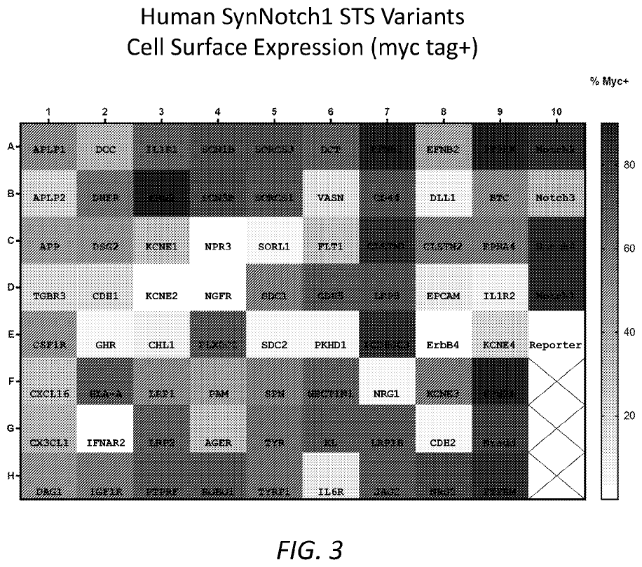

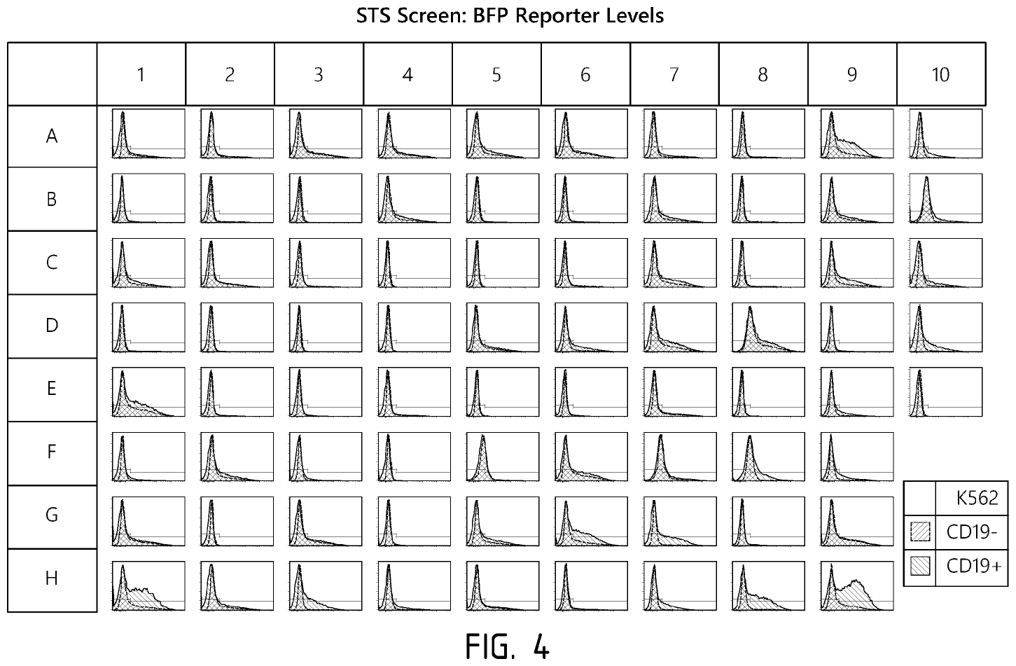

As described in Example 3 and FIGS. 2 and 3, chimeric polypeptide receptors have been tested and validated in primary human T cells. Without being bound to any particular theory, it is contemplated that these new receptors show similar performance in mouse models. The receptors disclosed herein may be engineered into various immune cell types for enhanced discrimination and elimination of tumors, or in engineered cells for control of autoimmunity and tissue regeneration. Accordingly, engineered cells, such as immune cells engineered to express one of more of the chimeric receptors disclosed herein, are also within the scope of the disclosure.

### Chimeric Polypeptides

This disclosure provides novel, non-naturally occurring recombinant chimeric polypeptides engineered to modulate transcriptional regulation in a ligand-dependent manner. In some embodiments, the receptors disclosed herein bind a target cell-surface displayed ligand, which triggers proteolytic cleavage of the receptors and release of a transcriptional regulator that modulates a custom transcriptional program in the cell.

In some embodiments, provided herein is a chimeric polypeptide including, from N-terminus to C-terminus (a) an extracellular ligand-binding domain (ECD) having a binding affinity for a selected ligand; (b) a linking polypeptide; (c) a transmembrane domain (TMD) including one or more ligand-inducible proteolytic cleavage sites; (d) a stop-transfer sequence (STS); and (e) an intracellular domain (ICD) including a transcriptional regulator, wherein binding of the selected ligand to the extracellular ligand-binding domain induces cleavage at one or more ligand-inducible proteolytic cleavage sites between the transcriptional regulator and the linking polypeptide, and wherein the STS is heterologous to the TMD. In some embodiments, the linking polypeptide has substantial sequence identity to the JMD of Notch1, Notch2, Notch3, and/or Notch4. In some embodiments, the linking polypeptide has substantial sequence identity to the Notch1, Notch2, Notch3, and/or Notch4 JMD, but does not include a LIN-12-Notch repeat (LNR) and/or a heterodimerization domain (HD) of a Notch receptor. In some embodiments, the linking polypeptide does not have substantial sequence identity to the Notch1, Notch2, Notch3, and/or Notch4 JMD. In some embodiments, the linking polypeptide includes an oligimerization domain which promotes formation of dimers, trimers, or higher order assemblages of the receptor.

In some embodiments, the STS is selected from the STS sequence in APLP1, APLP2, APP, TGBR3, CSF1R, CXCL16, CX3CL1, DAG1, DCC, DNER, DSG2, CDH1, GHR, HLA-A, IFNAR2, IGF1R, IL1R1, ERN2, KCNE1, KCNE2, CHL1, LRP1, LRP2, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, KL, IL6R, EFNB1, CD44, CLSTN1, LRP8, PCDHGC3, NRG1, LRP1B, JAG2, EFNB2, DLL1, CLSTN2, EPCAM, ErbB4, KCNE3, CDH2, NRG2, PTPRK, BTC, EPHA4, IL1R2, KCNE4, SCN2B, Nradd, PTPRM, Notch1, Notch2, Notch3, or Notch4. In some embodiments, the STS is selected from the STS sequence in DAG1, PTPRF, CDH5, KL, LRP1B, JAG2, KCNE3, NRG2, PTPRK, EPHA4, PTPRM, NOTCH1, NOTCH2, NOTCH3, NOTCH4, CXCL16, or NRG1.

### Extracellular Domains (ECD)

In some embodiments, the (ECD) of the chimeric polypeptides receptors disclosed herein has a binding affinity for one or more target ligands. The target ligand is expressed on a cell surface, or is otherwise immobilized or restrained to the extent that it can exert a mechanical force on the chimeric receptor. For example, an otherwise soluble ligand may be targeted if it is bound to a surface, or to a molecule in the extracellular matrix. In some embodiments, the target ligand is a cell-surface ligand. Non-limiting examples of suitable ligands include cell surface receptors; adhesion proteins; carbohydrates, lipids, glycolipids, lipoproteins, and lipopolysaccharides that are surface-bound; integrins; mucins; and lectins. In some embodiments, the ligand is a protein. In some embodiments, the ligand is a carbohydrate. In some embodiments, the ligand is a cluster of differentiation (CD) marker. In some embodiments, the CD marker is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof In some embodiments, the antigen-binding moiety is selected from the group consisting of an antibody, a nanobody, a diabody, a triabody, or a minibody, a F(ab′)₂ fragment, a Fab fragment, a single chain variable fragment (scFv), and a single domain antibody (sdAb), or a functional fragment thereof In some embodiments, the antigen-binding moiety includes an scFv.

The antigen-binding moiety can include naturally-occurring amino acid sequences or can be engineered, designed, or modified so as to provide desired and/or improved properties, e.g., increased binding affinity. Generally, the binding affinity of an antigen-binding moiety, e.g., an antibody, for a target antigen (e.g., CD19 protein) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding moiety that binds the antigen with high affinity and does not significantly bind other unrelated antigens.

A skilled artisan can select an extracellular domain based on the desired localization or function of a cell that is genetically modified to express an engineered receptor of the present disclosure. For example, an extracellular domain comprising an antibody specific for an estrogen receptor can target cells to estrogen-dependent breast cancer cells. In some embodiments, the extracellular domain of the disclosed engineered receptors is capable of binding a tumor associated-antigen (TAA) or a tumor-specific antigen (TSA). A skilled artisan in the art will understand that TAAs comprise molecules, e.g., proteins, present on tumor cells and on some fraction of normal cells, or on many normal cells but at much lower concentration than on tumor cells. Examples include, without limitation, CEA, AFP, HER2, CTAG1B and MAGEA1. In contrast, TSAs comprise molecules, e.g., proteins, present on tumor cells but absent from normal cells. Examples include, without limitation, oncoviral antigens and mutated proteins (also known as neoantigens).

In some cases, the antigen-binding moiety is specific for an epitope present in an antigen that is expressed by a tumor cell, i.e., a TSA or a TAA. The TSA or TAA can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a colorectal cancer cell, and the like. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

Non-limiting examples of suitable target antigens include CD19, B7H3 (CD276), BCMA (CD269), alkaline phosphatase, placental-like 2 (ALPPL2), green fluorescent protein (GFP), enhanced green fluorescent protein (EGFP), signal regulatory protein α (SIRPα), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SSEA-4, TAG72, TEM1CD248, TEM7R, TSHR, VEGFR2, ALPI, citrullinated vimentin, cMet, and Axl.

In some embodiments, the target antigen is selected from CD19, B7H3 (CD276), BCMA (CD269), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, Axl, GPC2, human epidermal growth factor receptor 2 (Her2/neu), CD276 (B7-H3), IL-13Rα1, IL-13Rα2, alphα-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNF-α, VEGF-A, pyruvate kinase isoenzyme type M2 (tumor M2-PK), CD20, CD5, CD7, CD3, TRBC1, TRBC2, CD38, CD123, CD93, CD34, CD1a, SLAMF7/CS1, FLT3, CD33, CD123, TALLA-1, CSPG4, DLL3, Kappa light chain, Lamba light chain, CD16/ FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin.

Further non-limiting examples of suitable antigens include PAP (prostatic acid phosphatase), prostate stem cell antigen (PSCA), prostein, NKG2D, TARP (T cell receptor γ alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), Ral-B, GPC2, CD276 (B7-H3), or IL-13Rα. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

Additional antigens that can be suitable for the chimeric polypeptide receptors disclosed herein include, but are not limited to GPC2, human epidermal growth factor receptor 2 (Her2/neu), CD276 (B7-H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional suitable antigens include, but are not limited to, those associated with an inflammatory disease such as, AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11a), myostatin, OX-40, scleroscin, SOST, TGFβ1, TNFα, and VEGF-A.

Further antigens suitable for the chimeric receptors disclosed herein include, but are not limited to the pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor y alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is GPC2, CD19, Her2/neu, CD276 (B7-H3), IL-13Rα1, or IL-13Rα2. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen-binding moiety include a LaG17 anti-GFP nanobody. In some embodiments, the antigen-binding moiety of the ECD includes an anti-BCMA fully humanized VH domain (FHVH).

In some embodiments, antigens suitable for targeting by the chimeric polypeptides and chimeric receptors disclosed herein include ligands derived from a pathogen. For example, the antigen can be HER2 produced by HER2-positive breast cancer cells. In some embodiments, the antigen can be CD19 that is expressed on B-cell leukemia. In some embodiments, the antigen can be EGFR that is expressed on glioblastoma multiform (GBM) but much less expressed so on healthy CNS tissue. In some embodiments, the antigen can be CEA that is associated with cancer in adults, for example colon cancer.

In some embodiments, the antigen-binding moiety of the extracellular domain is specific for a cell surface target, where non-limiting examples of cell surface targets include CD19, CD30, Her2, CD22, ENPP3, EGFR, CD20, CD52, CD11α, and α-integrin. In some embodiments, the chimeric receptors disclosed herein include an extracellular domain having an antigen-binding moiety that binds CD19, CEA, HER2, MUC1, CD20, or EGFR. In some embodiments, the chimeric receptors disclosed herein include an extracellular domain including an antigen-binding moiety that binds CD19. In some embodiments, the antigen- binding moiety includes an amino acid sequence having at least 70%, at least 75%, at least 80%, at least 85%, at least 90%, at least 95%, at least 96%, at least 97%, at least 98%, at least 99% sequence identity to SEQ ID NO: 199 in the Sequence Listing. In some embodiments, the antigen-binding moiety includes an amino acid sequence having at least 90% sequence identity to SEQ ID NO: 199. In some embodiments, the antigen-binding moiety includes an amino acid sequence having at least 95% sequence identity to SEQ ID NO: 199. In some embodiments, the antigen-binding moiety includes an amino acid sequence having 100% sequence identity to SEQ ID NO: 199. In some embodiments, the antigen-binding moiety includes an amino acid sequence having a sequence of SEQ ID NO: 199, wherein one, two, three, four, or five of the amino acid residues in SEQ ID NO: 199 is/are substituted by a different amino acid residue.

### Linking Polypeptide

The ECD and the TMD are linked to each other with a linking polypeptide. “SynNotch” receptors comprise a heterologous extracellular ligand-binding domain, a linking polypeptide having substantial sequence identity with a Notch receptor JMD (including the NRR), a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor JMD but lacking the NRR (the LIN-12-Notch repeat (LNR) modules, and the heterodimerization domain), a TMD, and an ICD. “Minimal Linker Notch” receptors comprise a heterologous extracellular ligand-binding domain, a linking polypeptide lacking substantial sequence identity with a Notch receptor (for example, without limitation, having a synthetic (GGS)ₙ polypeptide sequence), a TMD, and an ICD. “Hinge Notch” receptors comprise a heterologous extracellular ligand-binding domain, a hinge sequence comprising an oligomerization domain (i.e., a domain that promotes dimerization, trimerization, or higher order multimerization with a synthetic receptor and/or an existing host receptor), a TMD, and an ICD.

In the Mini Notch receptor, the linking polypeptide is derived from a Notch JMD sequence after deletion of the NRR and HD domain. The Notch JMD sequence may be the sequence from Notch1, Notch2, Notch3, or Notch4, and can be derived from a non-human homolog, such as those from Drosophila, Gallus, Danio, and the like. Four to 50 amino acid residues of the remaining Notch sequence (e.g., 4, 5, 6, 7, 8, 9, 10, 11, 12, 13, 14, 15, 16, 17, 18, 19, 20, or more, amino acid residues) can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide, such as the signal transduction level when ligand induced or in the absence of ligand. In some embodiments, the linker polypeptide sequence includes a sequence having at least 80% sequence identity, such as, at least 80%, at least 85%, at least 90%, at least 95%, at least 96%, at least 97%, at least 98%, or 99% sequence identity to a sequence selected from the group consisting of SEQ ID NOS: 23-33 in the Sequence Listing. In some embodiments, the linker polypeptide sequence includes an amino acid sequence having at least 90% sequence identity to a sequence selected from the group consisting of SEQ ID NOS: 23-33. In some embodiments, the linker polypeptide sequence includes an amino acid sequence having at least 95% sequence identity to a sequence selected from the group consisting of SEQ ID NOS: 23-33. In some embodiments, the linker polypeptide sequence includes an amino acid sequence having at least 100% sequence identity to a sequence selected from the group consisting of SEQ ID NOS: 23-33. In some embodiments, the linker polypeptide sequence includes an amino acid sequence having a sequence selected from the group consisting of SEQ ID NOS: 23-33, wherein one, two, three, four, or five of the amino acid residues in any one of the SEQ ID NOS: 23-33 is/are substituted by a different amino acid residue.

In the Minimal Linker Notch receptor, the linking polypeptide does not have substantial sequence identity to a Notch JMD sequence, including the Notch JMD sequence from Notch1, Notch2, Notch3, or Notch4, or a non-human homolog thereof. Four to 50 amino acid residues (e.g., 4, 5, 6, 7, 8, 9, 10, 11, 12, 13, 14, 15, 16, 17, 18, 19, 20, or more, amino acid residues) can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide of the disclosure. In some embodiments, the linker polypeptide includes a sequence having less than 80% sequence identity, such as, less than 80%, less than 75%, less than 70%, less than 65%, less than 60% sequence identity to a sequence selected from the group consisting of SEQ ID NOS: 23-33 in the Sequence Listing. In some embodiments, the linker polypeptide sequence includes an amino acid sequence having less than 90% sequence identity to a sequence selected from the group consisting of SEQ ID NOS: 23-33. In some embodiments, the linker polypeptide sequence includes an amino acid sequence having less than 95% sequence identity to a sequence selected from the group consisting of SEQ ID NOS: 23-33. In some embodiments, the linker polypeptide sequence includes an amino acid sequence having less than 100% sequence identity to a sequence selected from the group consisting of SEQ ID NOS: 23-33. The Minimal Linker sequence can be designed to include or omit a protease cleavage site, and can include or omit a glycosylation site or sites for other types of post-translational modification. In some embodiments, the Minimal Linker linker does not comprise a protease cleavage site or a glysosylation site.

Hinge linkers of the disclosure include an oligomerization domain (e.g., a hinge domain) containing one or more polypeptide motifs that promote oligomer formation of the chimeric polypeptides via intermolecular disulfide bonding. In these instances, within the chimeric Notch receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof In some embodiments, the hinge polypeptide sequence contains one or more CXXC motifs. In some embodiments, the hinge polypeptide sequence contains one or more CPPC motifs. Additional information in this regard can be found in, for example, a recent review by G. Vidarsson et al., Frontiers Immunol (2014) 5:520 (doi: 10.3389/fimmu.2014.00520), which is hereby incorporated by reference in its entirety.

Hinge polypeptide sequences can also be derived from a CD8α hinge domain, a CD28 hinge domain, a CD152 hinge domain, a PD-1 hinge domain, a CTLA4 hinge domain, an OX40 hinge domain, and functional variants thereof In some embodiments, the hinge domain includes a hinge polypeptide sequence derived from a CD8a hinge domain or a functional variant thereof. In some embodiments, the hinge domain includes a hinge polypeptide sequence derived from a CD28 hinge domain or a functional variant thereof In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The Hinge linker can include about 5 to about 60 amino acids from or overlapping with the selected hinge domain, for example at least about 5, at least about 6, at least about 7, at least about 8, at least about 9, at least about 10, at least about 12, at least about 15, at least about 17, at least about 20, at least about 22, at least about 24, at least about 26, at least about 28, at least about 30, at least about 35, at least about 40, at least about 45, at least about 50, at least about 55, or , at least about 60 amino acids. In embodiments of the invention, the Hinge linker has no more than about 60 amino acids, less than about 55, less than about 50, less than about 45, less than about 40, less than about 35, less than about 32, less than about 30, less than about 29, less than about 28, less than about 27, less than about 26, less than about 25, less than about 24, less than about 23, less than about 22, less than about 21, less than about 20, less than about 18, less than about 16, less than about 14, less than about 12, or less than about 10 amino acids.

The Fn Notch linking polypeptide is derived from the Robol JMD, which contains a fibronectin repeat (Fn) domain, with a short polypeptide sequence between the Fn repeats and the TMD. The Fn Notch linking polypeptide does not contain a Notch negative regulatory region (NRR), or the Notch HD domain. The Fn linking polypeptide can contain 1, 2, 3, 4, or 5 Fn repeats. In some embodiments, the chimeric receptor comprises a Fn linking polypeptide having about 1 to about 5 Fn repeats, about 1 to about 3 Fn repeats, or about 2 to about 3 Fn repeats. The short polypeptide sequence between the Fn repeats and the TMD can be from about 2 to about 30 amino acid residues (e.g., 2, 3, 4, 5, 6, 7, 8, 9, 10, 11, 12, 13, 14, 15, 16, 17, 18, 19, 20, or more, amino acid residues). In some embodiments, the short polypeptide sequence can be between about 5 and about 20 amino acids, of any sequence. In some embodiments, the short polypeptide sequence can be between about 5 and about 20 naturally-occurring amino acids, of any sequence. In some embodiments, the short polypeptide sequence can be between about 5 and about 20 amino acids, of any sequence but having no more than one proline. In some embodiments, the short polypeptide sequence can be between about 5 and about 20 amino acids, and about 50% or more of the amino acids are glycine. In some embodiments, the short polypeptide sequence can be between about 5 and about 20 amino acids, where the amino acids are selected from glycine, serine, threonine, and alanine. In some embodiments, the length and amino acid composition of the Fn linking polypeptide sequence can be varied to alter the orientation and/or proximity of the ECD and the TMD relative to one another to achieve a desired activity of the chimeric polypeptide of the disclosure. In some embodiments, the JMD polypeptide sequence includes a sequence having at least 80% sequence identity, such as, at least 80%, at least 85%, at least 90%, at least 95%, at least 96%, at least 97%, at least 98%, or 99% sequence identity to a sequence selected from the group consisting of SEQ ID NOS: 9-11 in the Sequence Listing. In some embodiments, the JMD polypeptide sequence includes an amino acid sequence having at least 90% sequence identity to a sequence selected from the group consisting of SEQ ID NOS: 9-11. In some embodiments, the Fn linker polypeptide sequence includes an amino acid sequence having at least 95% sequence identity to a sequence selected from the group consisting of SEQ ID NOS: 9-11. In some embodiments, the Fn linker polypeptide sequence includes an amino acid sequence having about 100% sequence identity to a sequence selected from the group consisting of SEQ ID NOS: 9-11. In some embodiments, the Fn linker polypeptide sequence includes an amino acid sequence having a sequence selected from the group consisting of SEQ ID NOS: 9-11, wherein one, two, three, four, or five of the amino acid residues in any one of the SEQ ID NOS: 9-11 is/are substituted by a different amino acid residue.

### Transmembrane Domains

As described above, the chimeric polypeptides of the disclosure include a TMD comprising one or more ligand-inducible proteolytic cleavage sites.

Examples of proteolytic cleavage sites in a Notch receptor (e.g., S2 or S3) are as described above. Additional proteolytic cleavage sites suitable for the compositions and methods disclosed herein include, but are not limited to, a metalloproteinase cleavage site for a MMP selected from collagenase-1, -2, and -3 (MMP-1, - 8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue such as Leu, Ile, Val, Phe, Trp, Tyr, Val, Met, and Pro) (SEQ ID NO: 211), e.g., Pro-X-X-Hy-(Ser/Thr) (SEQ ID NO: 212), e.g., Pro-Leu/Gln-Gly-Met-Thr-Ser (SEQ ID NO: 213) or Pro-Leu/Gln-Gly-Met-Thr (SEQ ID NO: 214). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Val-Gly-Arg (SEQ ID NO: 215). Another example of a protease cleavage site that can be included in a proteolytically cleavable linker is a tobacco etch virus (TEV) protease cleavage site, e.g., Glu-Asn-Leu-Tyr-Thr-Gln-Ser (SEQ ID NO: 216), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 217), where cleavage occurs after the lysine residue. Another example of a protease cleavage site that can be included in a proteolytically cleavable linker is a thrombin cleavage site, e.g., Leu-Val-Pro-Arg (SEQ ID NO: 218). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr virus proteas, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermolysin, fibroblast collagenase and stromelysin-1, matrix metalloproteinase 13 (collagenase-3), tissue-type plasminogen activator(tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

Generally, the TMD suitable for the chimeric receptors disclosed herein can be any transmembrane domain of a Type 1 transmembrane receptor including at least one γ-secretase cleavage site. Detailed description of the structure and function of the γ-secretase complex as well as its substrate proteins, including amyloid precursor protein (APP) and Notch, can, for example, be found in a recent review by Zhang et al., Frontiers Cell Neurosci (2014). Non-limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one γ-secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma In some embodiments, the TMD of the chimeric polypeptides or Notch receptors of the disclosure is a TMD known for Notch receptors. In some embodiments, the TMD of the chimeric polypeptides or Notch receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the TMD includes an amino acid sequence exhibiting at least 70%, at least 75%, at least 80%, at least 85%, at least 90%, at least 95%, at least 96%, at least 97%, at least 98%, at least 99% sequence identity to one or more of SEQ ID NOS: 181, 206, 241, and 242 in the Sequence Listing. In some embodiments, the TMD includes an amino acid sequence having at least 90% sequence identity to a sequence selected from the group consisting of SEQ ID NOS: 181, 206, 241, and 242. In some embodiments, the TMD includes an amino acid sequence having at least 95% sequence identity to a sequence selected from the group consisting of SEQ ID NOS: 181, 206, 241, and 242. In some embodiments, the TMD includes an amino acid sequence having 100% sequence identity to one or more of SEQ ID NOS: 181, 206, 241, and 242. In some embodiments, the TMD includes an amino acid sequence having a sequence selected from the group consisting of SEQ ID NOS: 181, 206, 241, and 242, wherein one, two, three, four, or five of the amino acid residues in any one of the SEQ ID NOS: 181, 206, 241, and 242 is/are substituted by a different amino acid residue.

In some embodiments, the amino acid substitution(s) within the TMD includes one or more substitutions within a “GV” motif of the TMD. In some embodiments, at least oone of such substitution(s) comprises a substitution to alanine. For example, one, two, three, four, five, or more of the amino acid residues of the sequence FMYVAAAAFVLLFFVGCGVLLS (SEQ ID NO: 181), as well as any one of the sequences set forth in SEQ ID NOS: 206, 241, and 242, may be substituted by a different amino acid residue. In some embodiments, the amino acid residue at position 18 and/or 19 of the “GV” motif within SEQ ID NO: 181 is substituted by a different amino acid residue. In some embodiments, the glycine residue at position 18 of SEQ ID NO: 181 is substituted by a different amino acid residue. In some embodiments, the valine residue at position 19 of SEQ ID NO: 181 is substituted by a different amino acid residue. In some embodiments, the transmembrane domain comprises an amino acid sequence having a sequence corresponding to SEQ ID NO: 181 with a mutation at the position corresponding to position 18 of SEQ ID NO: 181, such as G18A mutations. In some embodiments, the transmembrane domain comprises an amino acid sequence having a sequence corresponding to SEQ ID NO: 181 with a mutation at the position corresponding to position 19 of SEQ ID NO: 181, such as V19A mutations.

### Stop-Transfer Sequences (STS)

The chimeric polypeptides and receptors of the disclosure include an STS which comprises a charged, hydrophilic domain located between the TMD and the ICD. Without being bound to any particular theory, this domain disposed between the TMD and the ICD prevents the ICD from entering the plasma membrane. In some embodiments, a single-chain peptide comprising about 1 to about 40 amino acid residues (e.g., 1, 2, 3, 4, 5, 6, 7, 8, 9, 10, 11, 12, 13, 14, 15, 16, 17, 18, 19, 20, or more amino acid residues) in which most of the residues have charged side chains unde physiological conditions can be used as a STS. In short STS embodiments (e.g., less than about 6 amino acids), about 5 or 6 of the amino acids will have charged side chains. In some embodiments, the STS includes about 1 to 15, about 5 to 20, about 8 to 25, about 10 to 30, about 12 to 35, about 14 to 40, about 5 to 40, about 10 to 35, about 15 to 30, about 20 to 25, about 20 to 40, about 10 to 30, about 4 to 20, or about 5 to 25 amino acid residues. In some embodiments, the STS includes about 4 to 10, about 5 to 12, about 6 to 14, about 7 to 18, about 8 to 20, about 9 to 22, about 10 to 24, or about 11 to 26 amino acid residues. In some embodiments, the STS includes about 4 to 10 residues, such as, 4, 5, 6, 7, 8, 9, or 10 amino acid residues.

In some embodiments, the STS includes a sequence having at least about 80% sequence identity, such as, at least 80%, at least 85%, at least 90%, at least 95%, at least 96%, at least 97%, at least 98%, or 99% sequence identity to the STS domain of a Type 1 receptor. In some embodiments, the STS includes an amino acid sequence having at least 90% sequence identity to the STS domain of a Type 1 receptor. In some embodiments, the STS includes an amino acid sequence having at least about 90% sequence identity to SEQ ID NOS: 100-174 and 207. In some embodiments, the STS includes an amino acid sequence having at least about 95% sequence identity to SEQ ID NOS: 100-174 and 207. In some embodiments, the STS includes an amino acid sequence having about 100% sequence identity to SEQ ID NOS: 100-174 and 207. In some embodiments, the STS includes an amino acid sequence having at least about 90% sequence identity to SEQ ID NOS: 105, 107, 123, 143, 146, 153-155, 161, 163, 164, 166, 171, 172, 174, or 207. In some embodiments, the STS includes an amino acid sequence having at least about 95% sequence identity to SEQ ID NOS: 105, 107, 123, 143, 146, 153-155, 161, 163, 164, 166, 171, 172, 174, or 207. In some embodiments, the STS includes an amino acid sequence having about 100% sequence identity to SEQ ID NOS: 105, 107, 123, 143, 146, 153-155, 161, 163, 164, 166, 171, 172, 174, or 207. In some embodiments, the STS includes an amino acid sequence of SEQ ID NOS: 100-174 and 207, wherein one, two, three, four, or five of the amino acid residues in SEQ ID NOS: 100-174 and 207 is/are substituted by a different amino acid residue. In some embodiments, the STS includes an amino acid sequence of SEQ ID NOS: 105, 107, 123, 143, 146, 153-155, 161, 163, 164, 166, 171, 172, 174, or 207, wherein one, two, three, four, or five of the amino acid residues in SEQ ID NOS: 105, 107, 123, 143, 146, 153-155, 161, 163, 164, 166, 171, 172, 174, or 207 is/are substituted by a different amino acid residue. In some embodiments, the STS comprises a sequence comprising only Lys (K) or Arg (R) in the first 4 residues. In some embodiments, the STS comprises one, two, three, four, five, or more basic residues. In some embodiments, the STS comprises five, four, three, two, one, or zero aromatic residues or residues with hydrophobic and/or bulky side chains.

### Intracellular Domain (ICD)

The chimeric receptor of the disclosure includes a transcriptional regulator. The transcriptional regulator of the disclosure is a biochemical element that acts to promote or inhibit the transcription of a promoter-driven DNA sequence. Transcriptional regulators suitable for the compositions and methods of the disclosure can be naturally-occurring transcriptional regulators or can be engineered, designed, or modified so as to provide desired and/or improved properties, e.g., modulating transcription. In some embodiments, the transcriptional regulator directly regulates differentiation of the cell. In some embodiments, the transcriptional regulator indirectly modulates differentiation of the cell by modulating the expression of a second transcription factor. It will be understood by one having ordinary skill in the art that a transcriptional regulator can be a transcriptional activator or a transcriptional repressor. In some embodiments, the transcriptional regulator is a transcriptional repressor. In some embodiments, the transcriptional regulator is a transcriptional activator. In some embodiments, the transcriptional regulator can further include a nuclear localization signal. In some embodiments, the transcriptional regulator is selected from Gal4-VP16, Gal4-VP64, tetR-VP64, ZFHD1-VP64, Gal4-KRAB, and HAP1-VP16. In some embodiments, the transcriptional regulator is Gal4-VP64.

In some embodiments, the ICD includes a sequence having at least 80% sequence identity, such as, at least 80%, at least 85%, at least 90%, at least 95%, at least 96%, at least 97%, at least 98%, or 99% sequence identity to SEQ ID NO: 182 in the Sequence Listing. In some embodiments, the ICD includes an amino acid sequence having at least 90% sequence identity to SEQ ID NO: 182. In some embodiments, the ICD includes an amino acid sequence having at least 95% sequence identity to SEQ ID NO: 182. In some embodiments, the ICD includes an amino acid sequence having at least 100% sequence identity to SEQ ID NO: 182. In some embodiments, the ICD includes an amino acid sequence of SEQ ID NO: 182, wherein one, two, three, four, or five of the amino acid residues in SEQ ID NO: 182 is/are substituted by a different amino acid residue.

### Additional Domains

In some embodiments, the Notch extracellular domains located N-terminally to the TMD can further include an additional amino acid sequence or structural domain, for example a membrane localization signal such as a CD8A signal (SEQ ID NO: 176), a detectable marker such as a myc tag (SEQ ID NO: 177) or His tag, and the like.

In some embodiments, the chimeric polypeptide of the disclosure comprises: (a) a linking polypeptide comprising an amino acid sequence having at least 80% sequence identity to any one of SEQ ID NOS: 178-180 and 183-198; (b) a TMD comprising an amino acid sequence having at least 80% sequence identity to either of SEQ ID NO: 181 or 206; and (c) an STS comprising an amino acid sequence having at least 80% sequence identity to any one of SEQ ID NOS: 100-174 and 207. In some embodiments, the chimeric polypeptide of the disclosure comprises: (a) a linking polypeptide comprising an amino acid sequence having at least about 90% sequence identity to any one of SEQ ID NOS: 178-180 and 183-198; (b) a TMD comprising an amino acid sequence having at least 90% sequence identity to either of SEQ ID NOS: 181 or 206; and (c) an STS comprising an amino acid sequence having at least 90% sequence identity to any one of SEQ ID NOS: 100-174 and 207. In some embodiments, the chimeric polypeptide of the disclosure comprises: (a) a linking polypeptide comprising an amino acid sequence having at least 95% sequence identity to any one of SEQ ID NO: 178-180 and 183-198; (b) a TMD comprising an amino acid sequence having at least 95% sequence identity to any one of SEQ ID NOS: 181, 206, 241, and 242; and (c) an STS comprising an amino acid sequence having at least 95% sequence identity to SEQ ID NOs: 100-174 and 207.

In some embodiments, the chimeric polypeptide of the disclosure comprises: (a) a linking polypeptide comprising an amino acid sequence selected from the group consisting of SEQ ID NO: 178-180 and 183-198; (b) a TMD comprising an amino acid sequence selected from the group consisting of SEQ ID NOS: 181, 206, 241, and 242; and (c) an STS comprising an amino acid sequence selected from any one of SEQ ID NOs: 100-174 and 207.

In some embodiments, the chimeric polypeptide of the disclosure comprises: (a) a linking polypeptide comprising an amino acid sequence selected from the group consisting of SEQ ID NO: 178-180 and 183-198, wherein one, two, three, four, or five of the amino acid residues in any one of the SEQ ID NOS: 178-180 and 183-198 is/are substituted by a different amino acid residue; (b) a TMD comprising an amino acid sequence selected from the group consisting of SEQ ID NOS: 181, 206, 241, and 242, wherein one, two, three, four, or five of the amino acid residues in any one of the SEQ ID NOS: 181, 206, 241, and 242 is/are substituted by a different amino acid residue; and (c) an STS comprising an amino acid sequence of any one of SEQ ID NOs: 100-174 and 207, wherein one, two, three, four, or five of the amino acid residues in any one of SEQ ID NOs: 100-174 and 207 is/are substituted by a different amino acid residue.

In some embodiments, the chimeric receptor of the disclosure includes an amino acid sequence having at least about 80%, 90%, 95%, 96%, 97, 98%, 99%, or 100% sequence identity to a chimeric receptor disclosed herein. In some embodiments, provided herein are chimeric receptors including an amino acid sequence having at least about 80%, 90%, 95%, 96%, 97, 98%, 99%, or 100% sequence identity to any one of SEQ ID NOs: 1-99.

### Nucleic Acid Molecules

In one aspect, some embodiments disclosed herein relate to nucleic acid molecules that include nucleotide sequences encoding the chimeric polypeptides provided herein, including expression cassettes, and expression vectors containing these nucleic acid molecules operably linked to heterologous nucleic acid sequences such as, for example, regulatory sequences which facilitate in vivo expression of the receptor in a host cell.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence that encodes a chimeric polypeptide including, from N-terminus to C- terminus (a) an extracellular ligand-binding domain having a binding affinity for a selected ligand; (b) a linking polypeptide; (c) a TMD including one or more ligand-inducible proteolytic cleavage sites; and (d) an intracellular domain including a transcriptional regulator, wherein binding of the selected ligand to the extracellular ligand-binding domain induces cleavage at the ligand-inducible proteolytic cleavage site between the transcriptional regulator and the linking polypeptide, and wherein the chimeric polypeptide does not include a LIN-12-Notch repeat (LNR) and/or a heterodimerization domain (HD) of a Notch receptor.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. Thus, in some embodiments, an expression cassette of the disclosure include a coding sequence for the chimeric polypeptide as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus.

In some embodiments, provided herein are nucleic acid molecules encoding a polypeptide with an amino acid sequence having at least about 80%, 90%, 95%, 96%, 97, 98%, 99%, or 100% sequence identity to a chimeric receptor disclosed herein. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOS: 1-99 identified in the Sequence Listing.

The nucleic acid sequences encoding the chimeric receptors can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See, e.g, J. Sambrook & D.W. Russell (2012). Molecular Cloning: A Laboratory Manual (4th ed.). Cold Spring Harbor, NY: Cold Spring Harbor Laboratory and J. Sambrook & D.W. Russell (2001). Molecular Cloning: A Laboratory Manual (3rd ed.). Cold Spring Harbor, NY: Cold Spring Harbor Laboratory (jointly referred to herein as “Sambrook”); F.M. Ausubel (1987). Current Protocols in Molecular Biology. New York, NY: Wiley (including supplements through 2014); D.M. Bollag et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; K.B. Mullis et al., (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

DNA vectors can be introduced into eukaryotic cells via conventional transformation or transfection techniques. Suitable methods for transforming or transfecting host cells can be found in Sambrook et al. (2012) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, e.g., Gluzman (Ed.), Eukaryotic Viral Vectors, CSH Laboratory Press, Cold Spring Harbor, N.Y.). For example, a chimeric receptor as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but because the genetic code is degenerate encode the same polypeptide, e.g., an antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid is a ribonucleic acid (RNA), RNA can be produced, for example, by in vitro transcription.

### Recombinant Cells and Cell Cultures

The nucleic acid of the present disclosure can be introduced into a host cell, such as a human T lymphocyte, to produce a recombinant cell containing the nucleic acid molecule. Accordingly, some embodiments of the disclosure relate to a methods for making a recombinant cell, including (a) providing a cell capable of protein expression and (b) contacting the provided cell with a recombinant nucleic acid of the disclosure.

Introduction of the nucleic acid molecules of the disclosure into cells can be achieved by viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas9, or DNA-guided endonuclease genome editing NgAgo (Natronobacterium gregoryi Argonaute), or TALEN genome editing (transcription activator-like effector nucleases). In some embodiments, the nucleic acid molecule present in the recombinant host cell as a mini-circle expression vector for a stable or transient expression.

The nucleic acid molecules can be encapsulated in a viral capsid or a lipid nanoparticle. Alternatively, endonuclease polypeptide(s) can be delivered by viral or non-viral delivery vehicles known in the art, such as electroporation or lipid nanoparticles. For example, introduction of nucleic acids into cells may be achieved using viral transduction methods. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also amenable for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell, a cytotoxic T cell, or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis of a blood sample obtained from a human subject.

In some embodiments, the recombinant cell further includes a second nucleic acid molecule as disclosed herein, wherein the first nucleic acid molecule and the second nucleic acid molecule do not have the same sequence. In some embodiments, the recombinant cell further includes a second chimeric polypeptide as disclosed herein, wherein the first chimeric polypeptide and the second chimeric polypeptide do not have the same sequence. In some embodiments, the first chimeric polypeptide modulates the expression and/or activity of the second chimeric polypeptide.

In some embodiments, the recombinant cell further includes an expression cassette encoding a protein of interest operably linked to a promoter, wherein expression of the protein of interest is modulated by the chimeric receptor transcriptional regulator. In some embodiments, the protein of interest is heterologous to the recombinant cell. In principle, there are no particular limitations with regard to suitable proteins whose expression is modulated by the chimeric receptor transcriptional regulator. Exemplary protein types suitable for use with the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporters. In some embodiments, the immune cell receptor comprises a T-cell receptor (TCR). In some embodiments, the immune cell receptor comprises a chimeric antigen receptor (CAR). In some embodiments, the expression cassette encoding the protein of interest is incorporated into the same nucleic acid molecule that encodes the chimeric receptor of the disclosure. In some embodiments, the expression cassette encoding the protein of interest is incorporated into a second expression vector that is separate from the nucleic acid molecule encoding the chimeric receptor of the disclosure.

In another aspect, provided herein are various cell cultures including at least one recombinant cell as disclosed herein, and a culture medium. Generally, the culture medium can be any one of suitable culture media for the cell cultures described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., a carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the chimeric polypeptides and Notch receptors of the disclosure can also be administered by transfection or infection using methods known in the art, including but not limited to the methods described in McCaffrey et al. (Nature 418:6893, 2002), Xia et al. (Nature Biotechnol. 20:1006-10, 2002), or Putnam (Am. J. Health Syst. Pharm. 53:151-60, 1996, erratum at Am. J. Health Syst. Pharm. 53:325, 1996).

### METHODS OF THE DISCLOSURE

Administration of any one of the therapeutic compositions described herein, e.g., nucleic acids, recombinant cells, and pharmaceutical compositions, can be used to treat individuals in the treatment of relevant health conditions or diseases, such as cancers and chronic infections. In some embodiments, the nucleic acids, recombinant cells, and pharmaceutical compositions described herein can be incorporated into therapeutic agents for use in methods of treating an individual who has, who is suspected of having, or who may be at high risk for developing one or more autoimmune disorders or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Accordingly, in one aspect, some embodiments of the disclosure relate to methods for inhibiting an activity of a target cell in an individual, the methods include administering to the individual a first therapy including one or more of nucleic acids, recombinant cells, and pharmaceutical compositions as disclosed herein, wherein the first therapy inhibits an activity of the target cell. For example, an activity of the target cell may be inhibited if its proliferation is reduced, if its pathologic or pathogenic behavior is reduced, if its destroyed or killed, and the like. Inhibition includes a reduction of the measured quantity of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cell as disclosed herein, wherein the recombinant cell inhibits an activity of the target cell in the individual. Generally, the target cell of the disclosed methods can be any cell and can be, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In another aspect, some embodiments of the disclosure relate to methods for the treatment of a health condition (e.g., disease) in an individual in need thereof, the methods include administering to the individual a first therapy including one or more of chimeric polypeptides, Fn Notch receptors, nucleic acids, recombinant cells, and pharmaceutical compositions as disclosed herein, wherein the first therapy treats the health condition in the individual. In some embodiments, the methods include administering to the individual a first therapy including an effective number of the recombinant cell an effective number of the recombinant cell as disclosed herein, wherein the recombinant cell treats the health condition.

In another aspect, some embodiments of the disclosure relate to methods for assisting in the treatment of a health condition (e.g., disease) in an individual in need thereof, the methods including administering to the individual a first therapy including one or more of the nucleic acids, recombinant cells, and/or pharmaceutical compositions as disclosed herein, and a second therapy, wherein the first and second therapies together treat the health condition in the individual. In some embodiments, the methods include administering to the individual a first therapy including an effective number of the recombinant cells as disclosed herein, wherein the recombinant cells treat the health condition.

### Administration of Recombinant Cells Into an Individual

In some embodiments, the methods of the disclosure involve administering an effective amount or number the recombinants cells of the disclosure to an individual who is in need of such method. This administering step can be accomplished using any method of implantation known in the art. For example, the recombinants cells can be injected directly in the individual's blood or otherwise administered to the individual.

In some embodiments, the methods disclosed herein include administering, which can be interchangeably used with “introducing” and “transplanting,” recombinant cells into an individual, by a method or route that results in at least partial localization of the introduced cells at a desired site such that a desired effect(s) is produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the life time of the individual, i.e., long-term engraftment.

When provided prophylactically, the recombinant cells described herein can be administered to an individual in advance of any symptom of a disease or condition to be treated. Accordingly, in some embodiments the prophylactic administration of a recombinant stem cell population serves to prevent the occurrence of symptoms of the disease or condition.

When provided therapeutically in some embodiments, recombinant stem cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments described herein, the recombinant cells are expanded in culture prior to administration to an individual in need thereof.

In some embodiments, the delivery of a recombinant cell composition (e.g., a composition comprising a plurality of recombinant cells according to any of the cells described herein) into an individual by a method or route results in at least partial localization of the cell composition at a desired site. A cell composition can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation, and the like. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, in other words a population of recombinant cells are administered other than directly into a target site, tissue, or organ, such that it enters, instead, the individual's circulatory system and, thus, is subject to metabolism and other like processes.

The efficacy of a treatment having a composition for the treatment of a disease or condition can be determined by the skilled clinician. However, one skilled in the art will appreciate that a treatment is considered effective treatment if any one or all of the signs or symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of disease can be determined by the skilled clinician. However, a treatment is considered effective treatment if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments of the disclosed methods, the individual is a mammal. In some embodiments, the mammal is human. In some embodiments, the individual has or is suspected of having a health condition (e.g., disease) associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

### Additional Therapies

As discussed above, the recombinant cells, and pharmaceutical compositions described herein can be administered in combination with one or more additional therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti--cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

### Methods for Modulating an Activity of a Cell

In another aspect, provided herein are various methods for modulating an activity of a cell. The methods involve: (a) providing a recombinant cell of the disclosure, and (b) contacting it with a selected ligand, wherein binding of the selected ligand to the extracellular ligand-binding domain induces cleavage of a ligand-inducible proteolytic cleavage site and releases the transcriptional regulator, wherein the released transcriptional regulator modulates an activity of the recombinant cell. One skilled in the art upon reading the present disclosure will appreciate that the disclosed methods can be carried out in vivo, ex vivo, or in vitro.

Activities of a cell that can be modulated using a method of the present disclosure include, but are not limited to, expression of a selected gene of the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell.

In some embodiments, the released transcriptional regulator modulates expression of a gene product of the cell. In some embodiments, the released transcriptional regulator modulates expression of a heterologous gene product in the cell. A heterologous gene product is one that is not normally produced by the cell. For example, the cell can be genetically modified with a nucleic acid comprising a nucleotide sequence encoding the heterologous gene product.

In some embodiments, the heterologous gene product is a secreted gene product. In some embodiments, the heterologous gene product is a cell surface gene product. In some cases, the heterologous gene product is an intracellular gene product. In some embodiments, the released transcriptional regulator simultaneously modulates expression of two or more heterologous gene products in the cell.

In some embodiments, the heterologous gene product in the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor (TCR), a chimeric antigen receptor (CAR), a toxin, a toxin derived protein, a transcriptional regulator, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

In some embodiments, the released transcriptional regulator modulates differentiation of the cell, and wherein the cell is an immune cell, a stem cell, a progenitor cell, or a precursor cell.The chimeric receptors of the disclosure provide a higher degree of expression than a standard SynNotch receptor not having an STS substitution, when using identical binding domains and ICDs. Depending on the ligand/binding domain pair and their affinity, the chimeric receptor can provide expression enhancement of about 10%, about 15%, about 20%, about 25%, about 30%, about 35%, about 40%, about 45%, or about 50% higher than a corresponding SynNotch receptor. Further, chimeric receptors of the disclosure can provide different noise levels (lower or higher levels of expression in the absence of the selected ligand).

Additionally, the chimeric receptors of the disclosure can provide transcriptional regulation that responds to the degree of T cell activation, independent of ligand binding. This permits additional flexibility in use, for example in cases where it is desired to enhance or suppress a T cell response when activated despite the absence of the chimeric receptor ligand.

### SYSTEMS AND KITS

Also provided herein are systems and kits including the chimeric receptors, recombinant nucleic acids, recombinant cells, or pharmaceutical compositions provided and described herein as well as written instructions for making and using the same. For example, provided herein, in some embodiments, are systems and/or kits that include one or more of: an chimeric polypeptide as described herein, a chimeric receptor as described herein, a recombinant nucleic acids as described herein, a recombinant cell as described herein, or a pharmaceutical composition as described herein. In some embodiments, the systems and/or kits of the disclosure further include one or more syringes (including pre-filled syringes) and/or catheters (including pre-filled syringes) used to administer one any of the provided recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, killing a target cancer cell, or treating a health condition (e.g., disease) in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g. CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### EXAMPLE 1

### Generation of Sender Cells

This Example describes the generation of myelogenous leukemia cells expressing CD19 at levels equivalent toDaudi tumors, for use in activating and testing cells expressing the receptor constructs of the disclosure.

The cancer cell lines used were K562 myelogenous leukemia cells (ATCC #CCL-243) and Daudi B cell lymphoblasts (ATCC #CCL-213). The K562 cells were lentivirally transduced to stably express human CD19 at levels equivalent to Daudi tumors. CD19 levels were determined by staining the cells with α-CD19 APC (Biolegend® #302212). All cell lines were sorted for expression of the transgenes.

### EXAMPLE 2

### Primary Human T Cell Isolation and Culture

This Example describes the isolation and culture of primary human T cells that were subsequently used in various cell transduction experiments described in the Examples below.

In these experiments, primary CD4+ and CD8+ T cells were isolated from blood after apheresis and enriched by negative selection using human T cell isolation kits (human CD4+ or CD8+ enrichment cocktail; STEMCELL Technologies Cat #15062 and 15063). Blood was obtained from Blood Centers of the Pacific (San Francisco, CA) as approved by the University Institutional Review Board. T cells were cryopreserved in growth medium (RPMI-1640, UCSF cell culture core) with 20% human AB serum (Valley Biomedical Inc., #HP1022) and 10% DMSO. After thawing, T cells were cultured in human T cell medium containing X-VIVO^{TM} 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### EXAMPLE 3

### Stable Transduction of Human T cells with Lentiviral Vectors

The Example describes a general protocol used for lentiviral transduction of human T cells.

Generally, lentiviral vectors pseudo-typed with vesicular stomatitis virus envelope G protein (VSV-G) (pantropic vectors) were produced via transfection of Lenti-X™ 293T cells (Clontech #11131D) with a pHR'SIN:CSW (K.T. Roybal et al., Cell (2016) 167(2):419-32) transgene expression vector and the viral packaging plasmids pCMVdR8.91 and pMD2.G using Mirus TransIT®-Lenti (Mirus, #MIR 6606). Generally, primary T cells were thawed the same day and, after 24 hours in culture, were stimulated with beads having anti-CD3 and anti-CD28 antibodies bound to the surface (Human T-Activator CD3/CD28 Dynabeads®, Life Technologies #11131D) at a 1:3 cell:bead ratio. At 48 hours, viral supernatant was harvested and the primary T cells were exposed to the virus for 24 hours. At Day 5 post T cell stimulation, the beads were removed, and the T cells expanded until Day 14 when they were rested and could be used in assays. T cells were sorted for assays with a Beckton Dickinson (BD Biosciences) FACSAria™ II flow cytometer. AND-gate T cells exhibiting basal CAR expression were gated out during sorting.

### EXAMPLE 4

### Design and Construction of Chimeric Receptor Constructs

This Example describes the design and construction of a family of synthetic receptors having heterologous STS sequences. Detailed information for various exemplary receptors of the disclosure can be found in Table 1 below.

Receptors were built by fusing a CD19 scFv to the corresponding receptor scaffold and GAL4 DBD VP64. All receptors contain an N-terminal CD8α signal peptide (SEQ ID NO: 176) for membrane targeting, and a myc-tag (SEQ ID NO: 177) for determining surface expression with an antibody conjugated to a fluorescent dye (α-myc A647®, cell-signaling #2233). The receptors were cloned into a modified pHR'SIN:CSW vector containing a PGK promoter for all primary T cell experiments.

DNA fragments coding for the amino acid sequences listed in Table 1 were PCR-amplified from either plasmids or synthesized gene fragments containing the corresponding DNA sequences, and assembled using standard cloning techniques (overhang PCR, fusion PCR, and In-fusion cloning) into a lentiviral expression vector (pHR-SIN-pGK, SEQ ID NO: 210) (see, e.g., K.T. Roybal et al., Cell (2016) 167(2):419-32.e16).

Each plasmid encodes a chimeric receptor with an extracellular domain (ECD, SEQ ID NO: 175) having, from N- to C-terminal, a CD8A signal peptide (MALPVTALLLPLALLLHAARP, SEQ ID NO: 176), a myc-tag (EQKLISEEDL, SEQ ID NO: 177), and an anti- CD19 scFv (D.L. Porter et al., N Engl J Med (2011) 365(8):725-33). C-terminal to the ECD, each chimeric receptor then has a extracellular linking polypeptide selected from the SynNotch JMD including the NRR (SynNotch, SEQ ID NO: 178, plasmids pSTS001 through pSTS072 and pIZ618 through pIZ620); a Mini Notch linking polypeptide excluding the NRR (Mini Notch, SEQ ID NO: 179, plasmids pSTS106 through pSTS172), and a Hinge Notch linking polypeptide (Hinge Notch, SEQ ID NO: 190, pSTS206 through pSTS272). Each of the constructs herein includes a Notch1 TMD (SEQ ID NO: 181), the STS indicated in Table 1, and a Gal4-VP64 transcriptional regulator (ICD, SEQ ID NO: 182).

Table 1 identifies the plasmid, receptor type, SEQ ID NO: for the full receptor, the selection of linking polypeptide (LP), the STS employed, and the SEQ ID NO: for the STS.

TABLE 1
Chimeric Receptors with Heterologous Stop Transfer Sequence

| | | FULL | | | STS |
|---|---|---|---|---|---|
| Plasmid ID | Receptor | SEQ ID | LP | STS | SEQ ID |
| pSTS00l | SynNotch1 with | 1 | SynNotch1 | RRKK | 100 |
| | APLPI STS | | | | |
| pSTS002 | Human SynNotch1 | 2 | SynNotch1 | RKR | 101 |
| | with APLP2 STS | | | | |
| pSTS003 | Human SynNotch1 | 3 | SynNotch1 | KKK | 102 |
| | with APP STS | | | | |
| pSTS004 | Human SynNotch1 | 4 | SynNotch1 | H | 103 |
| | with TGBR3 STS | | | | |
| pSTS005 | Human SynNotch1 | 5 | SynNotch1 | KYKQKPK | 104 |
| | with CSF1R STS | | | | |
| pSTS006 | Human SynNotch1 | 6 | SynNotch1 | KRRR | 105 |
| | with CXCL16 STS | | | | |
| pSTS007 | Human SynNotch1 | 7 | SynNotch1 | RK | 106 |
| | with CX3CL1 STS | | | | |
| pSTS008 | Human SynNotch1 | 8 | SynNotch1 | RKKRKGK | 107 |
| | with DAG1 STS | | | | |
| pSTS009 | Human SynNotch1 | 9 | SynNotch1 | RR | 108 |
| | with DCC STS | | | | |
| pSTS010 | Human SynNotch1 | 10 | SynNotch1 | RISR | 109 |
| | with DNER STS | | | | |
| pSTS011 | Human SynNotch1 | 11 | SynNotch1 | KCGKGAK | 110 |
| | with DSG2 STS | | | | |
| pSTS012 | Human SynNotch1 | 12 | SynNotch1 | RRR | 111 |
| | with CDH1 STS | | | | |
| pSTS013 | Human SynNotch1 | 13 | SynNotch1 | KQQRIK | 112 |
| | with GHR STS | | | | |
| pSTS014 | Human SynNotch1 | 14 | SynNotch1 | RRK | 113 |
| | with HLA-A STS | | | | |
| pSTS015 | Human SynNotch1 | 15 | SynNotch1 | K | 114 |
| | with IFNAR2 STS | | | | |
| pSTS016 | Human SynNotch1 | 16 | SynNotch1 | HRKR | 115 |
| | with IGFIR STS | | | | |
| pSTS017 | Human SynNotch1 | 17 | SynNotch1 | KIFK | 116 |
| | with IL1R1 STS | | | | |
| pSTS018 | Human SynNotch1 | 18 | SynNotch1 | R | 117 |
| | with ERN2 STS | | | | |
| pSTS019 | Human SynNotch1 | 19 | SynNotch1 | RSKKLEH | 118 |
| | with KCNEl STS | | | | |
| pSTS020 | Human SynNotch1 | 20 | SynNotch1 | KSKRREH | 119 |
| | with KCNE2 STS | | | | |
| pSTS021 | Human SynNotch1 | 21 | SynNotch1 | KRNRGGK | 120 |
| | with CHLI STS | | | | |
| pSTS022 | Human SynNotch1 | 22 | SynNotch1 | KRR | 121 |
| | with LRPI STS | | | | |
| pSTS023 | Human SynNotch1 | 23 | SynNotch1 | HYRR | 122 |
| | with LRP2 STS | | | | |
| pSTS024 | Human SynNotch1 | 24 | SynNotch1 | KRKRTH | 123 |
| | with PTPRF STS | | | | |
| pSTS025 | Human SynNotch1 | 25 | SynNotch1 | KK | 124 |
| | with SCNIB STS | | | | |
| pSTS026 | Human SynNotch1 | 26 | SynNotch1 | RKVSK | 125 |
| | with SCN3B STS | | | | |
| pSTS027 | Human SynNotch1 | 27 | SynNotch1 | RKKYR | 126 |
| | with NPR3 STS | | | | |
| pSTS028 | Human SynNotch1 | 28 | SynNotch1 | KR | 127 |
| | with NGFR STS | | | | |
| pSTS029 | Human SynNotch1 | 29 | SynNotch1 | HH | 128 |
| | with PLXDC2 STS | | | | |
| pSTS030 | Human SynNotch1 | 30 | SynNotch1 | RWKKSR | 129 |
| | with PAM STS | | | | |
| pSTS031 | Human SynNotch1 | 31 | SynNotch1 | RRQRR | 130 |
| | with AGER STS | | | | |
| pSTS032 | Human SynNotch1 | 32 | SynNotch1 | RHRKKR | 131 |
| | with ROBO1 STS | | | | |
| pSTS033 | Human SynNotch1 | 33 | SynNotch1 | KRK | 132 |
| | with SORCS3 STS | | | | |
| pSTS034 | Human SynNotch1 | 34 | SynNotch1 | KFKRR | 133 |
| | with SORCS1 STS | | | | |
| pSTS035 | Human SynNotch1 | 35 | SynNotch1 | KHRR | 134 |
| | with SORL1 STS | | | | |
| pSTS036 | Human SynNotch1 | 36 | SynNotch1 | RMKKK | 135 |
| | with SDC1 STS | | | | |
| pSTS037 | Human SynNotch1 | 37 | SynNotch1 | RMRKK | 136 |
| | with SDC2 STS | | | | |
| pSTS038 | Human SynNotch1 | 38 | SynNotch1 | RRRQKRR | 137 |
| | with SPN STS | | | | |
| pSTS039 | Human SynNotch1 | 39 | SynNotch1 | RHKRK | 138 |
| | with TYR STS | | | | |
| pSTS040 | Human SynNotch1 | 40 | SynNotch1 | RARR | 139 |
| | with TYRP1 STS | | | | |
| pSTS041 | Human SynNotch1 | 41 | SynNotch1 | RRLRK | 140 |
| | with DCT STS | | | | |
| pSTS042 | Human SynNotch1 | 42 | SynNotch1 | RRGR | 141 |
| | with VASN STS | | | | |
| pSTS043 | Human SynNotch1 | 43 | SynNotch1 | RKMKR | 142 |
| | with FLT1 STS | | | | |
| pSTS044 | Human SynNotch1 | 44 | SynNotch1 | RRRLRKQARAHGK | 143 |
| | with CDH5 STS | | | | |
| pSTS045 | Human SynNotch1 | 45 | SynNotch1 | KRSKSRKTK | 144 |
| | with PKHD1 STS | | | | |
| pSTS046 | Human SynNotch1 | 46 | SynNotch1 | RRRRHTFK | 145 |
| | with NECTIN1 STS | | | | |
| pSTS047 | Human SynNotch1 | 47 | SynNotch1 | KKGRRSYK | 146 |
| | with KL STS | | | | |
| pSTS048 | Human SynNotch1 | 48 | SynNotch1 | RFKKTWKLRAL- | 147 |
| | with IL6R STS | | | KEGK | |
| pSTS049 | Human SynNotch1 | 49 | SynNotch1 | I<LRI<RHRI<H | 148 |
| | with EFNBI STS | | | | |
| pSTS050 | Human SynNotch1 | 50 | SynNotch1 | RRRCGQKKK | 149 |
| | with CD44 STS | | | | |
| pSTS051 | Human SynNotch1 | 51 | SynNotch1 | RIRAAHRRTMR | 150 |
| | with CLSTN1 STS | | | | |
| pSTS052 | Human SynNotch1 | 52 | SynNotch1 | RNWKRKNTK | 151 |
| | with LRP8 STS | | | | |
| pSTS053 | Human SynNotch1 | 53 | SynNotch1 | KVYKWKQSR | 152 |
| | with PCDHGC3 STS | | | | |
| pSTS054 | Human SynNotch1 | 54 | SynNotch1 | KTKKQRKKLHDRLR | 153 |
| | with NRG1 STS | | | | |
| pSTS055 | Human SynNotch1 | 55 | SynNotch1 | KRKRRTKTIRR | 154 |
| | with LRP1B STS | | | | |
| pSTS056 | Human SynNotch1 | 56 | SynNotch1 | RKRRKERERSRLPR | 155 |
| | with JAG2 STS | | | | |
| pSTS057 | Human SynNotch1 | 57 | SynNotch1 | KYRRRHRKH | 156 |
| | with EFNB2 STS | | | | |
| pSTS058 | Human SynNotch1 | 58 | SynNotch1 | RLRLQKHR | 157 |
| | with DLL1 STS | | | | |
| pSTS059 | Human SynNotch1 | 59 | SynNotch1 | RVRIAHQH | 158 |
| | with CLSTN2 STS | | | | |
| pSTS060 | Human SynNotch1 | 60 | SynNotch1 | RKKRMAKYEK | 159 |
| | with EPCAM STS | | | | |
| pSTS061 | Human SynNotch1 | 61 | SynNotch1 | RRKSIKKKRALRR | 160 |
| | with ErbB4 STS | | | | |
| pSTS062 | Human SynNotch1 | 62 | SynNotch1 | RSRKVDKR | 161 |
| | with KCNE3 STS | | | | |
| pSTS063 | Human SynNotch1 | 63 | SynNotch1 | KRRDKERQAK | 162 |
| | with CDH2 STS | | | | |
| pSTS064 | Human SynNotch1 | 64 | SynNotch1 | KTKKQRKQ- | 163 |
| | with NRG2 STS | | | MHNHLR | |
| pSTS065 | Human SynNotch1 | 65 | SynNotch1 | KKSKLAKKRK | 164 |
| | with PTPRK STS | | | | |
| pSTS066 | Human SynNotch1 | 66 | SynNotch1 | HPLRKRRKRKKK | 165 |
| | with BTC STS | | | | |
| pSTS067 | Human SynNotch1 | 67 | SynNotch1 | RRRSKYSKAK | 166 |
| | with EPHA4 STS | | | | |
| pSTS068 | Human SynNotch1 | 68 | SynNotch1 | HRRCKHRTGK | 167 |
| | with IL1R2 STS | | | | |
| pSTS069 | Human SynNotch1 | 69 | SynNotch1 | KSKRREKK | 168 |
| | with KCNE4 STS | | | | |
| pSTS070 | Human SynNotch1 | 70 | SynNotch1 | KCVRRKKEQK | 169 |
| | with SCN2B STS | | | | |
| pSTS071 | Human SynNotch1 | 71 | SynNotch1 | KCWRSHKQR | 170 |
| | with Nradd STS | | | | |
| pSTS072 | Human SynNotch1 | 72 | SynNotch1 | KKRKLAKKRK | 171 |
| | with PTPRM STS | | | | |
| pIZ618 | Human SynNotch1 | 73 | SynNotch1 | KRKRKH | 172 |
| | with Notch2 STS | | | | |
| pIZ619 | Human SynNotch1 | 74 | SynNotch1 | RRKREH | 173 |
| | with Notch3 STS | | | | |
| pIZ620 | Human SynNotch1 | 75 | SynNotch1 | RRRRREH | 174 |
| | with Notch4 STS | | | | |
| pSTS106 | miniNotch with | 76 | Mini Notch | KRRR | 105 |
| | CXCL16 STS | | | | |
| pSTS108 | miniNotch with | 77 | Mini Notch | RKKRKGK | 107 |
| | DAG1 STS | | | | |
| pSTS113 | miniNotch with | 78 | Mini Notch | KQQRIK | 112 |
| | GHRSTS | | | | |
| pSTS124 | miniNotch with | 79 | Mini Notch | KRKRTH | 123 |
| | PTPRF STS | | | | |
| pSTS147 | miniNotch with KL | 80 | Mini Notch | KKGRRSYK | 146 |
| | STS | | | | |
| pSTS154 | miniNotch with | 81 | Mini Notch | KTKKQRKKLHDRLR | 153 |
| | NRG1 STS | | | | |
| pSTS155 | miniNotch with | 82 | Mini Notch | KRKRRTKTIRR | 154 |
| | LRP1B STS | | | | |
| pSTS156 | miniNotch with | 83 | Mini Notch | RKRRKERERSRLPR | 155 |
| | JAG2 STS | | | | |
| pSTS160 | miniNotch with | 84 | Mini Notch | RKKRMAKYEK | 159 |
| | EPCAM STS | | | | |
| pSTS164 | miniNotch with | 85 | Mini Notch | KTKKQRKQ- | 163 |
| | NRG2 STS | | | MHNHLR | |
| pSTS165 | miniNotch with | 86 | Mini Notch | KKSKLAKKRK | 164 |
| | PTPRK STS | | | | |
| pSTS172 | miniNotch with | 87 | Mini Notch | KKRKLAKKRK | 171 |
| | PTPRM STS | | | | |
| pSTS206 | HingeNotch with | 88 | Hinge Notch | KRRR | 105 |
| | CXCL16 STS | | | | |
| pSTS208 | HingeNotch with | 89 | Hinge Notch | RKKRKGK | 107 |
| | DAG1 STS | | | | |
| pSTS213 | HingeNotch with | 90 | Hinge Notch | KQQRIK | 112 |
| | GHR STS | | | | |
| pSTS224 | HingeNotch with | 91 | Hinge Notch | KRKRTH | 123 |
| | PTPRF STS | | | | |
| pSTS247 | HingeNotch with | 92 | Hinge Notch | KKGRRSYK | 146 |
| | KLSTS | | | | |
| pSTS254 | HingeNotch with | 93 | Hinge Notch | KTKKQRKKLHDRLR | 153 |
| | NRG1 STS | | | | |
| pSTS255 | HingeNotch with | 94 | Hinge Notch | KRKRRTKTIRR | 154 |
| | LRP1B STS | | | | |
| pSTS256 | HingeNotch with | 95 | Hinge Notch | RKRRKERERSRLPR | 155 |
| | JAG2 STS | | | | |
| pSTS260 | HingeNotch with | 96 | Hinge Notch | RKKRMAKYEK | 159 |
| | EPCAM STS | | | | |
| pSTS264 | HingeNotch with | 97 | Hinge Notch | KTKKQRK- | 163 |
| | NRG2 STS | | | QMHNHLR | |
| pSTS265 | HingeNotch with | 98 | Hinge Notch | KKSKLAKKRK | 164 |
| | PTPRK STS | | | | |
| pSTS272 | HingeNotch with | 99 | Hinge Notch | KKRKLAKKRK | 171 |
| | PTPRM STS | | | | |

The synthetic receptors were then expressed and assayed for activity as described in the Examples herein. The expression heat map for SynNotch1 receptors is shown in FIG. 3 (receptors are listed below according to their positions in the heat map).

| | | | | | | | | | |
|---|---|---|---|---|---|---|---|---|---|
| APLP1 | DCC | IL1R1 | SCN1B | SORCS3 | DCT | EFNB1 | EFNB2 | PTPRK | Notch2 |
| APLP2 | DNER | ERN2 | SCN3B | SORCS1 | VASN | CD44 | DLL1 | BTC | Notch3 |
| APP | DSG2 | KCNE1 | NPR3 | SORL1 | FLT1 | CLSTN1 | CLSTN2 | EPHA4 | Notch4 |
| TGBR3 | CDH1 | KCNE2 | NGFR | SDC1 | CDH5 | LRP8 | EPCAM | IL1R2 | Notch1 |
| CSF1R | GHR | CHL1 | PLXDC2 | SDC2 | PKHD1 | PCDHGC3 | ErbB4 | KCNE4 | Reporter |
| CXCL16 | HLA-A | LRP1 | PAM | SPN | NECTIN1 | NRG1 | KCNE3 | SCN2B | N/A |
| CX3CL1 | IFNAR2 | LRP2 | AGER | TYR | KL | LRP1B | CDH2 | Nradd | N/A |
| DAG1 | IGF1R | PTPRF | ROBO1 | TYRP1 | IL6R | JAG2 | NRG2 | PTPRM | N/A |

### EXAMPLE 5

### Reporter Expression in Jukat T cells

This Example describes the generation and screening of reporter Jurkat T cells that were used for the screening of heterologous STSs.

E6-1 reporter-positive Jurkat T cells (1×10⁵, ATCC# TIB-152) were lentivirally transduced with the receptor variants in 96 well plates. Transduced Jurkat T cells were co-cultured with ligand-expressing K562 cancer cells at a 1:1 ratio in round bottom 96-well tissue culture plates. The cultures were analyzed at 24 hours for surface receptor expression via antibody staining for the extracellular myc-tag and BFP reporter expression with a BD Fortessa X-50. Corrected percent BFP positivity was calculated by dividing the percent BFP-positive for the mixed Jurkat T cell population by the percent myc-positive. A receptor with a given STS was designated as a “hit” if it demonstrated a corrected BFP-positivity of at least 10 percent and a percent myc-positive of at least 5 percent, and “not a hit” if it did not meet both criteria. Functional STS receptor variants, along with a selection of negative hits, were confirmed in human primary T cells using the above protocols. The results are shown in Table 2.

TABLE 2
Results of Heterologous STS Construct Signal Transduction

| | | | STS | |
|---|---|---|---|---|
| Plasmid | Receptor | STS sequence | SEQ ID | Results |
| pSTS001 | SynNotch1 with | RRKK | 100 | Not a Hit: Low Fold-change |
| | APLP1 STS | | | and Low % Change |
| pSTS002 | SynNotch1 with | RKR | 101 | Not a Hit: Low receptor |
| | APLP2 STS | | | expression |
| pSTS003 | SynNotch1 with | KKK | 102 | Not a Hit: Low Fold-change |
| | APP STS | | | and Low % Change |
| pSTS004 | SynNotch1 with | H | 103 | Not a Hit: Low receptor |
| | TGBR3 STS | | | expression |
| pSTS005 | SynNotch1 with | KYKQKPK | 104 | Not a Hit: Low Fold-change |
| | CSF1R STS | | | and Low % Change |
| pSTS006 | SynNotch1 with | KRRR | 105 | Not a Hit: Low % Change |
| | CXCL16 STS | | | |
| pSTS007 | SynNotch1 with | RK | 106 | Not a Hit: Low Fold-change |
| | CX3CL1 STS | | | and Low % Change |
| pSTS008 | SynNotch1 with | RKKRKGK | 107 | Hit |
| | DAG1 STS | | | |
| pSTS009 | SynNotch1 with | RR | 108 | Not a Hit: Low Fold-change |
| | DCC STS | | | and Low % Change |
| pSTS010 | SynNotch1 with | RISR | 109 | Not a Hit: Low Fold-change |
| | DNER STS | | | and Low % Change |
| pSTS011 | SynNotch1 with | KCGKGAK | 110 | Not a Hit: Low Fold-change |
| | DSG2 STS | | | and Low % Change |
| pSTS012 | SynNotch1 with | RRR | ill | Not a Hit: Low receptor |
| | CDH1 STS | | | expression |
| pSTS013 | SynNotch1 with | KQQRIK | 112 | Not a Hit: Low receptor |
| | GHR STS | | | expression |
| pSTS014 | SynNotch1 with | RRK | 113 | Not a Hit: Low Fold-change |
| | HLA-A STS | | | and Low % Change |
| pSTS015 | SynNotch1 with | K | 114 | Not a Hit: Low receptor |
| | IFNAR2 STS | | | expression |
| pSTS016 | SynNotch1 with | HRKR | 115 | Not a Hit: Low Fold-change |
| | IGF1R STS | | | and Low % Change |
| pSTS017 | SynNotch1 with | KIFK | 116 | Not a Hit: Low Fold-change |
| | ILIRI STS | | | and Low % Change |
| pSTS018 | SynNotch1 with | R | 117 | Not a Hit: Low Fold-change |
| | ERN2 STS | | | and Low % Change |
| pSTS019 | SynNotch1 with | RSKKLEH | 118 | Not a Hit: Low Fold-change |
| | KCNE1 STS | | | and Low % Change |
| pSTS020 | SynNotch1 with | KSKRREH | 119 | Not a Hit: Low receptor |
| | KCNE2 STS | | | expression |
| pSTS021 | SynNotch1 with | KRNRGGK | 120 | Not a Hit: Low receptor |
| | CHL1 STS | | | expression |
| pSTS022 | SynNotch1 with | KRR | 121 | Not a Hit: Low % Change |
| | LRP1 STS | | | |
| pSTS023 | SynNotch1 with | HYRR | 122 | Not a Hit: Low Fold-change |
| | LRP2 STS | | | and Low % Change |
| pSTS024 | SynNotch1 with | KRKRTH | 123 | Hit |
| | PTPRF STS | | | |
| pSTS025 | SynNotch1 with | KK | 124 | Not a Hit: Low Fold-change |
| | SCN1B STS | | | and Low % Change |
| pSTS026 | SynNotch1 with | RKVSK | 125 | Not a Hit: Low Fold-change |
| | SCN3B STS | | | and Low % Change |
| pSTS027 | SynNotch1 with | RKKYR | 126 | Not a Hit: Low receptor |
| | NPR3 STS | | | expression |
| pSTS028 | SynNotch1 with | KR | 127 | Not a Hit: Low receptor |
| | NGFR STS | | | expression |
| pSTS029 | SynNotch1 with | HH | 128 | Not a Hit: Low Fold-change |
| | PLXDC2 STS | | | and Low % Change |
| pSTS030 | SynNotch1 with | RWKKSR | 129 | Not a Hit: Low Fold-change |
| | PAM STS | | | and Low % Change |
| pSTS031 | SynNotch1 with | RRQRR | 130 | Not a Hit: Low % Change |
| | AGER STS | | | |
| pSTS032 | SynNotch1 with | RHRKKR | 131 | Not a Hit: Low % Change |
| | ROBO1 STS | | | |
| pSTS033 | SynNotch1 with | KRK | 132 | Not a Hit: Low Fold-change |
| | SORCS3 STS | | | and Low % Change |
| pSTS034 | SynNotch1 with | KFKRR | 133 | Not a Hit: Low Fold-change |
| | SORCS1 STS | | | and Low % Change |
| pSTS035 | SynNotch1 with | KHRR | 134 | Not a Hit: Low receptor |
| | SORL1 STS | | | expression |
| pSTS036 | SynNotch1 with | RMKKK | 135 | Not a Hit: Low Fold-change |
| | SDC1 STS | | | and Low % Change |
| pSTS037 | SynNotch1 with | RMRKK | 136 | Not a Hit: Low receptor |
| | SDC2 STS | | | expression |
| pSTS038 | SynNotch1 with | RRRQKRR | 137 | Not a Hit: Low Fold-change |
| | SPN STS | | | and Low % Change |
| pSTS039 | SynNotch1 with | RHKRK | 138 | Not a Hit: Low Fold-change |
| | TYR STS | | | and Low % Change |
| pSTS040 | SynNotch1 with | RARR | 139 | Not a Hit: Low Fold-change |
| | TYRP1 STS | | | and Low % Change |
| pSTS041 | SynNotch1 with | RRLRK | 140 | Not a Hit: Low Fold-change |
| | DCT STS | | | and Low % Change |
| pSTS042 | SynNotch1 with | RRGR | 141 | Not a Hit: Low receptor |
| | VASN STS | | | expression |
| pSTS043 | SynNotch1 with | RKMKR | 142 | Not a Hit: Low Fold-change |
| | FLTI STS | | | and Low % Change |
| pSTS044 | SynNotch1 with | RRRLRKQARA- | 143 | Hit |
| | CDH5 STS | HGK | | |
| pSTS045 | SynNotch1 with | KRSKSRKTK | 144 | Not a Hit: Low receptor |
| | PKHD1 STS | | | expression |
| pSTS046 | SynNotch1 with | RRRRHTFK | 145 | Not a Hit: Low Fold-change |
| | NECTIN1 STS | | | and Low % Change |
| pSTS047 | SynNotch1 with KL | KKGRRSYK | 146 | Hit |
| | STS | | | |
| pSTS048 | SynNotch1 with | RFKKTWKLRA- | 147 | Not a Hit: Low receptor |
| | IL6R STS | LKEGK | | expression |
| pSTS049 | SynNotch1 with | KLRKRHRKH | 148 | Not a Hit: Low % Change |
| | EFNB1 STS | | | |
| pSTS050 | SynNotch1 with | RRRCGQKKK | 149 | Not a Hit: Low Fold-change |
| | CD44 STS | | | and Low % Change |
| pSTS051 | SynNotch1 with | RIRAAHRRTMR | 150 | Not a Hit: Low Fold-change |
| | CLSTN1 STS | | | and Low % Change |
| pSTS052 | SynNotch1 with | RNWKRKNTK | 151 | Not a Hit: Low Fold-change |
| | LRP8 STS | | | and Low % Change |
| pSTS053 | SynNotch1 with | KVYKWKQSR | 152 | Not a Hit: Low Fold-change |
| | PCDHGC3 STS | | | and Low % Change |
| pSTS054 | SynNotch1 with | KTKKQRKKLH- | 153 | Not a Hit: Low receptor |
| | NRG1 STS | DRLR | | expression |
| pSTS055 | SynNotch1 with | KRKRRTKTIRR | 154 | Hit |
| | LRP1B STS | | | |
| pSTS056 | SynNotch1 with | RKRRKERERS- | 155 | Hit |
| | JAG2 STS | RLPR | | |
| pSTS057 | SynNotch1 with | KYRRRHRKH | 156 | Not a Hit: Low Fold-change |
| | EFNB2 STS | | | and Low % Change |
| pSTS058 | SynNotch1 with | RLRLQKHR | 157 | Not a Hit: Low receptor |
| | DLL1 STS | | | expression |
| pSTS059 | SynNotch1 with | RVRIAHQH | 158 | Not a Hit: Low Fold-change |
| | CLSTN2 STS | | | and Low % Change |
| pSTS060 | SynNotch1 with | RKKRMAKYEK | 159 | Not a Hit: Low receptor |
| | EPCAM STS | | | expression |
| pSTS061 | SynNotch1 with | RRKSIKKKRA- | 160 | Not a Hit: Low receptor |
| | ErbB4 STS | LRR | | expression |
| pSTS062 | SynNotch1 with | RSRKVDKR | 161 | Hit |
| | KCNE3 STS | | | |
| pSTS063 | SynNotch1 with | KRRDKERQAK | 162 | Not a Hit: Low receptor |
| | CDH2 STS | | | expression |
| pSTS064 | SynNotch1 with | KTKKQRKQM- | 163 | Hit |
| | NRG2 STS | HNHLR | | |
| pSTS065 | SynNotch1 with | KKSKLAKKRK | 164 | Hit |
| | PTPRK STS | | | |
| pSTS066 | SynNotch1 with | HPLRKRRKRK- | 165 | Not a Hit: Low Fold-change |
| | BTC STS | KK | | and Low % Change |
| pSTS067 | SynNotch1 with | RRRSKYSKAK | 166 | Hit |
| | EPHA4 STS | | | |
| pSTS068 | SynNotch1 with | HRRCKHRTGK | 167 | Not a Hit: Low receptor |
| | IL1R2 STS | | | expression |
| pSTS069 | SynNotch1 with | KSKRREKK | 168 | Not a Hit: Low receptor |
| | KCNE4 STS | | | expression |
| pSTS070 | SynNotch1 with | KCVRRKKEQK | 169 | Not a Hit: Low % Change |
| | SCN2B STS | | | |
| pSTS071 | SynNotch1 with | KCWRSHKQR | 170 | Not a Hit: Low Fold-change |
| | Nradd STS | | | and Low % Change |
| pSTS072 | SynNotch1 with | KKRKLAKKRK | 171 | Hit |
| | PTPRM STS | | | |
| pIZ618 | SynNotch1 with | KRKRKH | 172 | Hit |
| | Notch2 STS | | | |
| pIZ619 | SynNotch1 with | RRKREH | 173 | Not a Hit: Low % Change |
| | Notch3 STS | | | |
| pIZ620 | SynNotch1 with | RRRRREH | 174 | Hit |
| | Notch4 STS | | | |
| pSTS106 | miniNotch with | KRRR | 105 | 2:1 Signal to Noise with 52% |
| | CXCL16 STS | | | BFP+ |
| pSTS108 | miniNotch with | RKKRKGK | 107 | (not done) |
| | DAG1 STS | | | |
| pSTS113 | miniNotch with | KQQRIK | 112 | 6.5:5.5 Signal to Noise with |
| | GHR STS | | | 64% BFP+, Poor S:N |
| pSTS124 | miniNotchwith | KRKRTH | 123 | (not done) |
| | PTPRF STS | | | |
| pSTS147 | miniNotch with KL | KKGRRSYK | 146 | (not done) |
| | STS | | | |
| pSTS154 | miniNotch with | KTKKQRKKLH- | 153 | 4:1 Signal to Noise with 42% |
| | NRG1 STS | DRLR | | BFP+ |
| pSTS155 | miniNotch with | KRKRRTKTIRR | 154 | 4:1 Signal to Noise with 64% |
| | LRPIB STS | | | BFP+ |
| pSTS156 | miniNotch with | RKRRKERERS- | 155 | 2.4:1 Signal to Noise with |
| | JAG2 STS | RLPR | | 62% BFP+ |
| pSTS160 | miniNotch with | RKKRMAKYEK | 159 | 1:1 Signal to Noise with 80% |
| | EPCAM STS | | | BFP+ Poor S:N |
| pSTS164 | miniNotch with | KTKKQRKQM- | 163 | 2.5:1 Signal to Noise with |
| | NRG2 STS | HNHLR | | 59% BFP+ |
| pSTS165 | miniNotch with | KKSKLAKKRK | 164 | 7:6 Signal to Noise with 72% |
| | PTPRK STS | | | BFP+ |
| pSTS172 | miniNotch with | KKRKLAKKRK | 171 | 3:2 Signal to Noise with 65% |
| | PTPRM STS | | | BFP+ |
| pSTS206 | HingeNotch with | KRRR | 105 | 50:1 Signal to Noise with |
| | CXCL16 STS | | | 31% BFP+ |
| pSTS208 | HingeNotch with | RKKRKGK | 107 | 3:1 Signal to Noise with 60% |
| | DAG1 STS | | | BFP+, Poor S:N for HingeNotch |
| pSTS213 | HingeNotch with | KQQRIK | 112 | 2:1 Signal to Noise with 21% |
| | GHR STS | | | BFP+, Poor S:N |
| pSTS224 | HingeNotch with | KRKRTH | 123 | 82:1 Signal to Noise with |
| | PTPRF STS | | | 58% BFP+ |
| pSTS247 | HingeNotch with KL | KKGRRSYK | 146 | 5:1 Signal to Noise with 67% |
| | STS | | | BFP+, Poor S:N for HingeNotch |
| pSTS254 | HingeNotch with | KTKKQRKKLH- | 153 | 85:1 Signal to Noise with |
| | NRG1 STS | DRLR | | 26% BFP+, |
| pSTS255 | HingeNotch with | KRKRRTKTIRR | 154 | 66:1 Signal to Noise with |
| | LRPIB STS | | | 53% BFP+, |
| pSTS256 | HingeNotch with | RKRRKERERS- | 155 | 49:1 Signal to Noise with |
| | JAG2 STS | RLPR | | 49% BFP+, |
| pSTS260 | HingeNotch with | RKKRMAKYEK | 159 | 2:1 Signal to Noise with 62% |
| | EPCAM STS | | | BFP+, PoorS:N |
| pSTS264 | HingeNotch with | KTKKQRKQM- | 163 | 75:1 Signal to Noise with |
| | NRG2 STS | HNHLR | | 45% BFP+, |
| pSTS265 | HingeNotch with | KKSKLAKKRK | 164 | 4.5:1 Signal to Noise with |
| | PTPRK STS | | | 47% BFP+, Poor S:N for |
| | | | | HingeNotch |
| pSTS272 | HingeNotch with | KKRKLAKKRK | 171 | 8:1 Signal to Noise with 39% |
| | PTPRM STS | | | BFP+, Poor S:N for |
| | | | | HingeNotch |

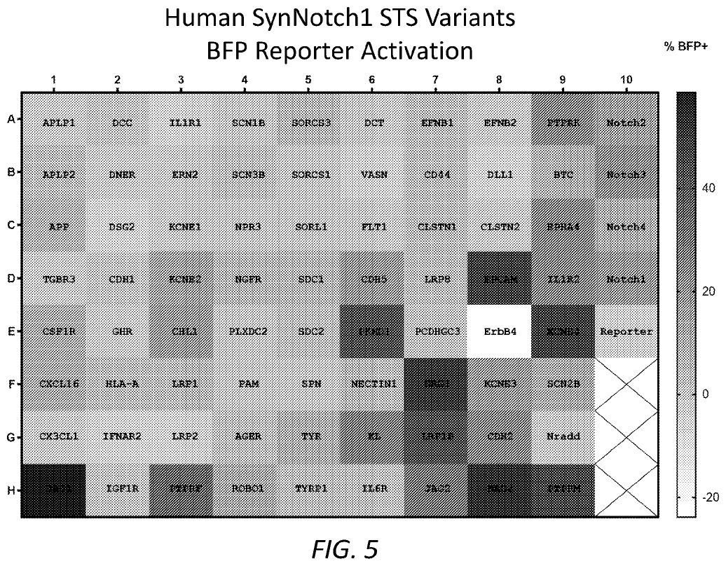

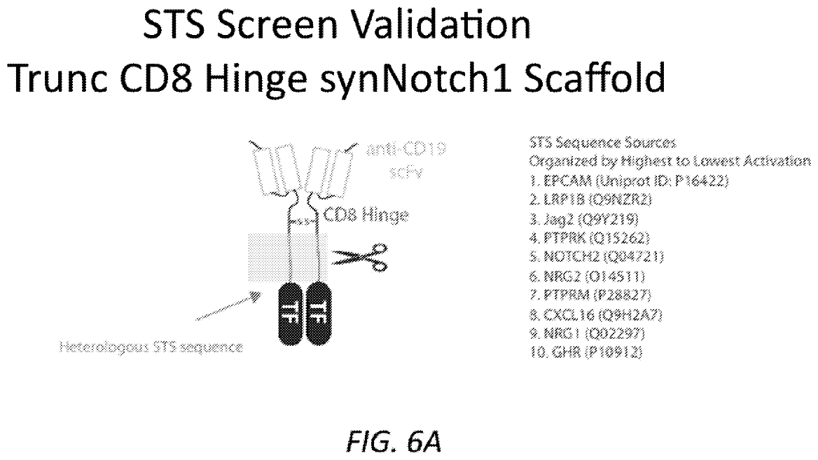

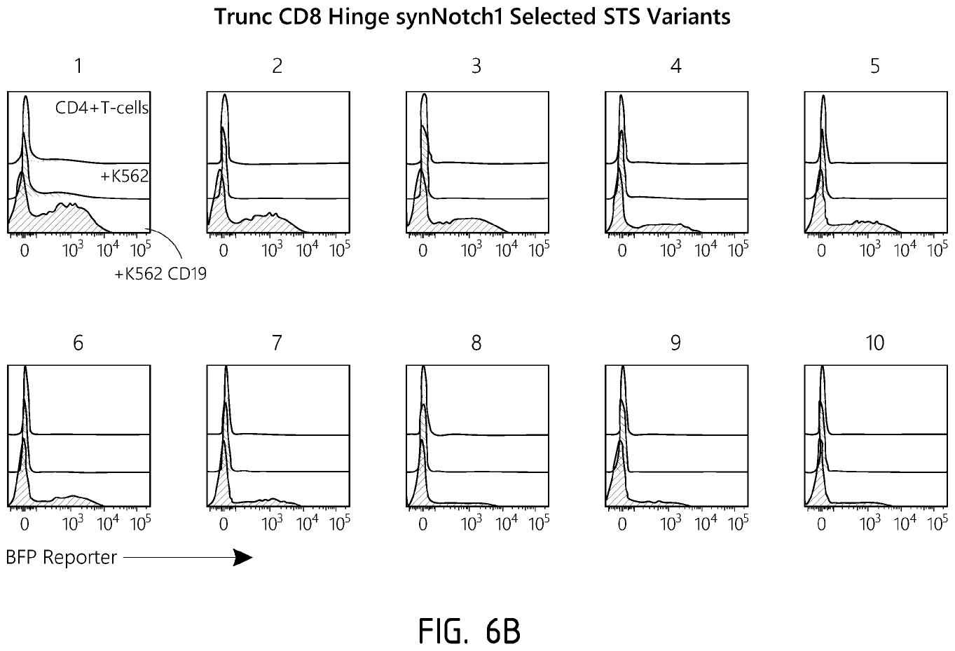

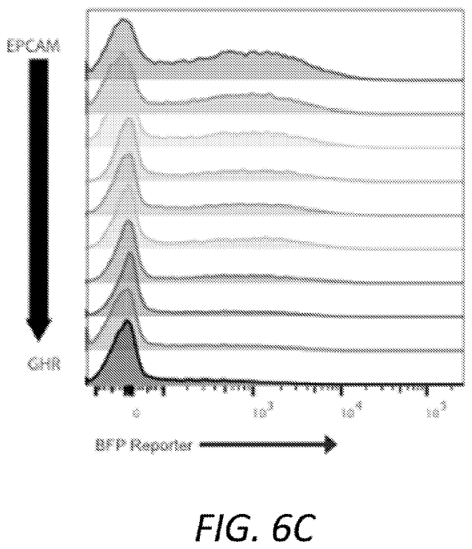

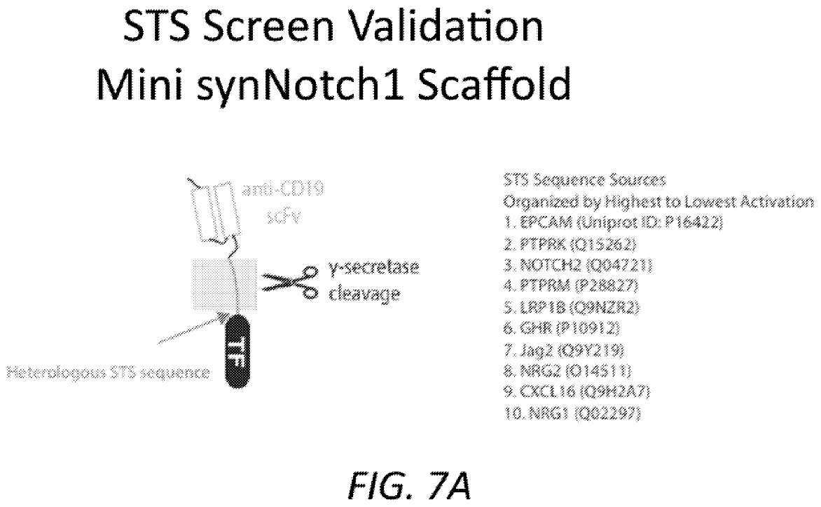

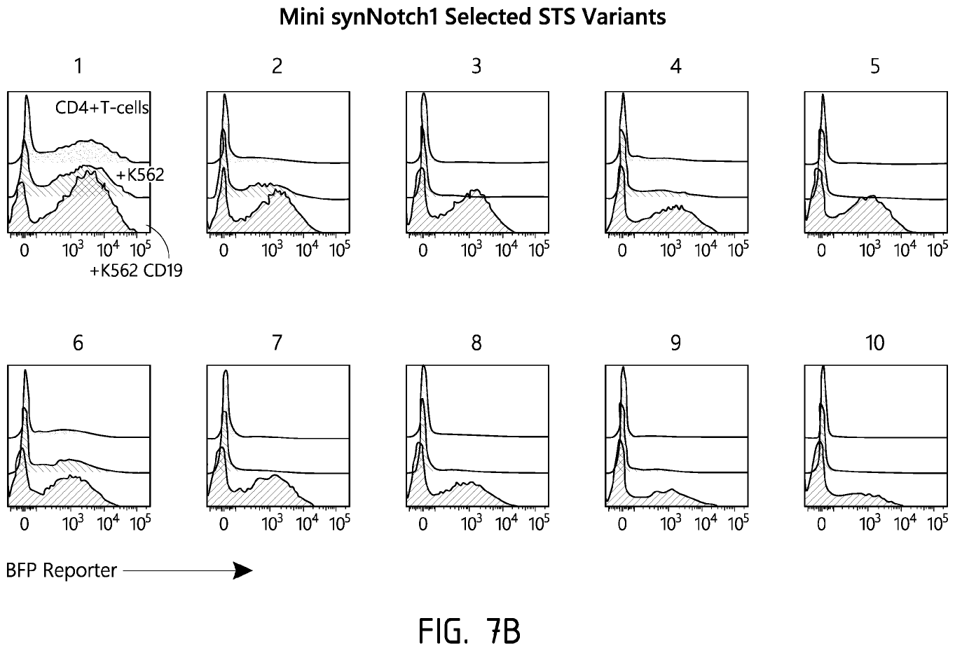

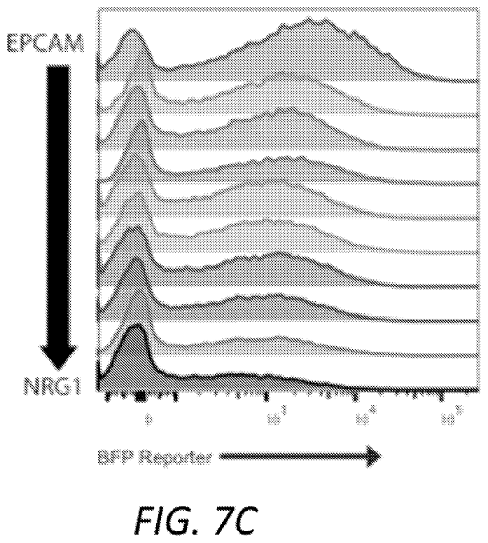

The results for SynNotch receptors are also shown in FIG. 5. Results for Hinge Notch receptors are shown in FIGS. 6B and 6C. Results for Mini Notch receptors are shown in FIGS. 7B and 7C. The results demonstrate that a variety of different STS sequences can be used in synthetic receptors of the disclosure, and that the choice of STS affects the expression response and signal to noise ratio of the receptor. This provides a palette of response characteristics that is of use to the synthetic receptor designer.

### EXAMPLE 6

### Comparison of Receptors Having a Notch1 or Notch2 STS

This experiment demonstrates the effect of using a heterologous Notch1 or Notch2 STS or TMD in a Mini Notch receptor.

Three Mini Notch receptors were constructed: miniNotch(2,2,2) (SEQ ID NO: 199), having a Notch2 JMD with the NRR deleted (SEQ ID NO: 183), a Notch2 TMD (SEQ ID NO: 206), and a Notch2 STS (SEQ ID NO: 172); miniNotch(2,1,1) (SEQ ID NO: 200), having a Notch2 JMD with the NRR deleted (SEQ ID NO: 183), a Notch1 TMD (SEQ ID NO: 181), and a Notch1 STS (SEQ ID NO: 207); and miniNotch(2,1,2) (SEQ ID NO: 201), having a Notch2 JMD with the NRR deleted (SEQ ID NO: 183), a Notch1 TMD (SEQ ID NO: 181), and a Notch2 STS (SEQ ID NO: 172). Jurkat cells were transduced using a lentiviral vector as described in the Examples above.

Expression of BFP was measured in the transduced Jurkat cells was measured (a) in the absence of sender cells, (b) in the presence of K562 sender cells without CD19, and (c) in the presence of K562 sender cells expressing CD19.

The results showed that miniNotch(2,2,2) was relatively noisy, demonstrating an expression level of greater than 50% in the absence of ligand, with relatively high expression in the presence of ligand. The second receptor, miniNotch(2,1,1) was very quiet, demonstrating an undetectable expression level in the absence of ligand, with relatively high expression in the presence of ligand (although less than the stimulated expression of miniNotch(2,2,2)). The third receptor, miniNotch(2,1,2) was intermediate in noise between miniNotch(2,2,2) and miniNotch(2,1,1), and equal in expression to miniNotch(2,2,2).

### EXAMPLE 7

### Further Comparison

Additional receptors with different linking sequences were constructed for further comparison.

The following constructs were prepared: SynNotch(1,1,1) (SEQ ID NO: 202), having a Notch1 STS (SEQ ID NO: 207); SynNotch(1,1,2) (SEQ ID NO: 203), having a Notch2 STS (SEQ ID NO: 172); miniNotch(1,1,1) (SEQ ID NO: 208), having a Notch1 STS (SEQ ID NO: 207); miniNotch(1,1,2) (SEQ ID NO: 209), having a Notch2 STS (SEQ ID NO: 172); truncHingeNotchNotch (SEQ ID NO: 204), having a Notch1 STS (SEQ ID NO: 207); and truncHingeNotchNotch(N2 STS) (SEQ ID NO: 205), having a Notch2 STS (SEQ ID NO: 172). The constructs were transduced into Jurkat cells and assayed as described above.

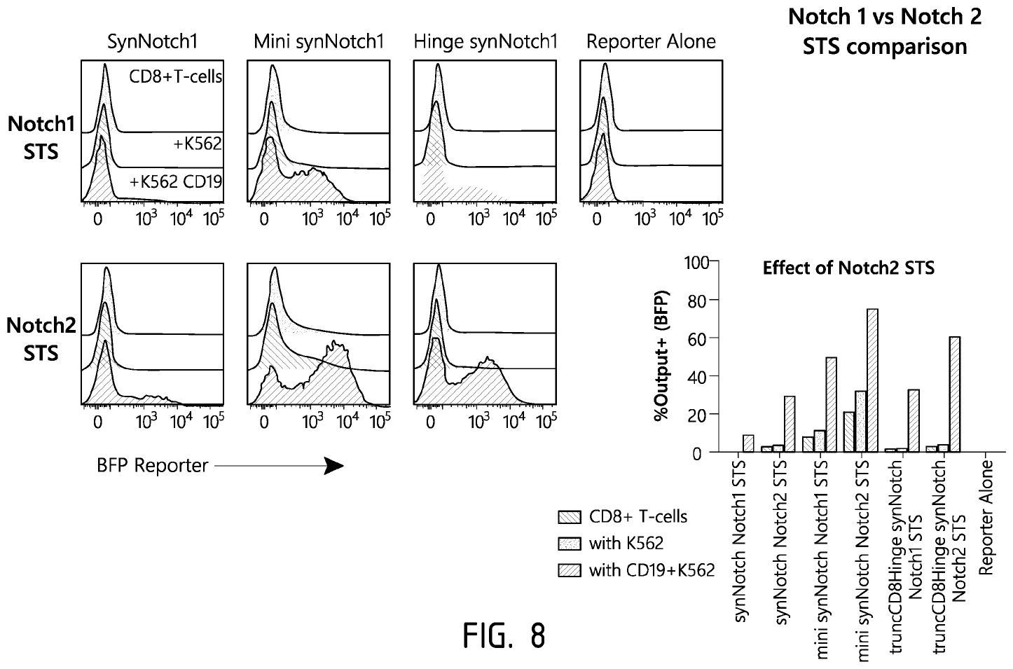

The results are shown in FIG. 8. SynNotch(1,1,2) was slightly noisier, but provided much higher expression than SynNotch(1,1,1). MiniNotch(1,1,1) was somewhat noisier than the first two receptors, but also achieved still higher expression. MiniNotch(1,1,2) was noisier than the first three receptors, but provided the highest expression level when stimulated. The fifth receptor, truncHingeNotch exhibited a noise level less than SynNotch(1,1,2), with an equivalent stimulated expression level. The last receptor, truncHingeNotch(N2 STS) provided an expression level second only to the MiniNotch(1,1,2), with a much lower noise level.

### EXAMPLE 8

This Example describes experiments performed to demonstrate activation of Hinge-Notch constructs with different ligand-binding domains and their dependence on proteolytic activity of ADAM proteases and gamma-secretase.

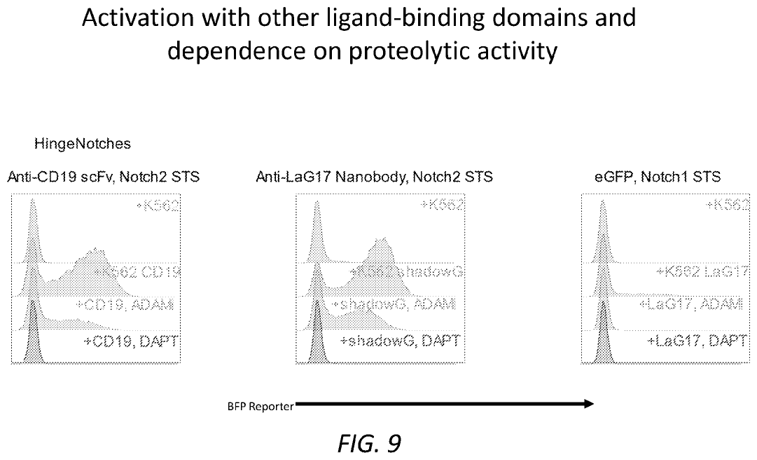

Three exemplary Hinge-Notch constructs were prepared, including one construct having an anti-CD19 scFv as the ligand recognition domain and a Notch2 STS domain, another construct having an anti-LaG17 nanobody as the ligand recognition domain and a Notch2 STS domain, and a third construct having an eGFP extracellular domain and a Notch1 STS domain. Primary human CD4+ T-cells were activated with anti-CD3/anti-CD28 Dynabeads (Gibco) and transduced with two lentiviral constructs, one expressing a hinge receptor with indicated binding head truncation variant receptor, and the other a transcriptional reporter (FIG. 9). Cells containing both constructs were sorted on Day 5 post initial T-cell stimulation and expanded further for activation testing. For testing, 1×10⁵ double positive T-cells expressing receptors were co-cultured with 1×10⁵ K562 cells (top trace), 1×10⁵ Ligand+K562 cells (second trace from top), 1×10⁵ Ligand+K562 cells with an ADAM10 inhibitor (third trace from top), or 1×10⁵ Ligand+K562 cells with a gamma-secretase inhibitor, DAPT (bottom trace). Transcriptional activation of an inducible BFP reporter gene was subsequently measured using a Fortessa X-50 (BD Biosciences).

### EXAMPLE 9

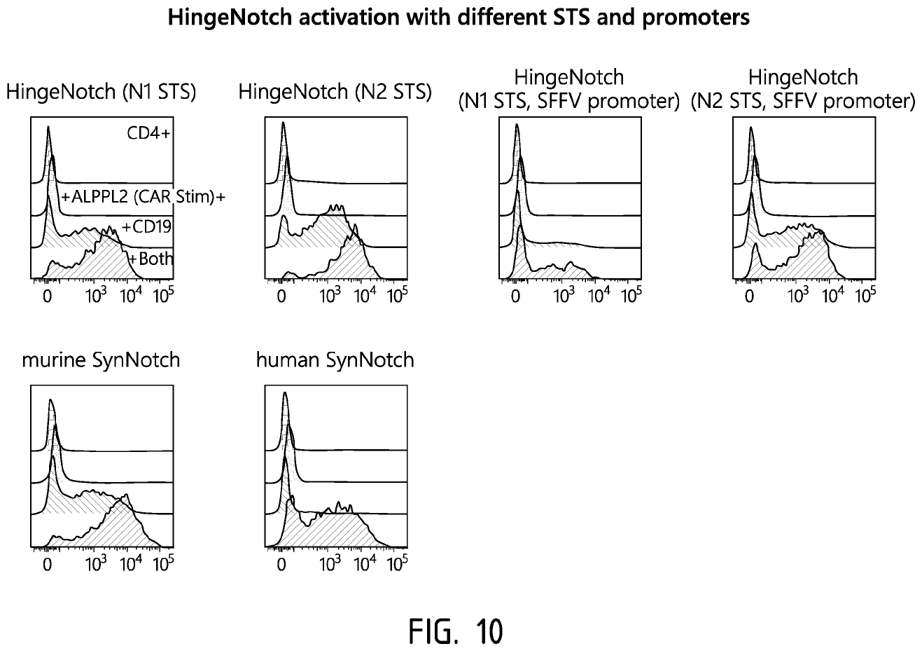

This Example describes experiments performed to compare activation of Hinge-Notch variants with different promoters and STS domains. For testing, 1×10⁵ double positive T-cells expressing anti-CD19 receptors were co-cultured with no additions (top trace), 1×10⁵ ALPPL2+ K562 cells (second trace from top), 1×10⁵ CD19+ K562 cells (third trace from top), or 1×10⁵ ALPPL2+ CD19+ K562 cells (bottom trace) (FIG. 10). Transcriptional activation of an inducible BFP reporter gene was subsequently measured using a Fortessa X-50 (BD Biosciences). Activation using murine and human original synNotch constructs were included for comparison.

### EXAMPLE 10

This Example describes Table 3 of activation characteristics of HingeNotch STS variants without additional T cell stimulation.

Primary human CD4+ T-cells were activated with anti-CD3/anti-CD28 Dynabeads (Gibco) and transduced with two lentiviral constructs, one expressing a hinge receptor, and the other a transcriptional reporter with constitutive eGFP-tagged anti-ALPPL2 CAR expression. Cells containing both constructs were sorted for Day 5 post initial T-cell stimulation and expanded further for activation testing. For testing, T-cells expressing receptors were co-cultured with K562 cells or CD19+ K562 cells. BFP reporter gene was subsequently measured using a Fortessa X-50 (BD Biosciences). Signal to noise ratios from the MFIs of BFP+ cells under CD19+ K562 versus K562 conditions are plotted against the delta change in MFI in the two conditions, as in Table 3 below.

TABLE 3
This table provides data without stimulation of co-expressed
anti-ALPPL2 CAR with ALPPL2+ K562. “Reporter alone” represents
a reporter plasmid and was expressed in all samples.

| | | | | Average MFI |
|---|---|---|---|---|
| | Recptor from | | | Difference |
| Plasmid | which STS is | | Average Signal | (MFI of ON- |
| ID | derived | STS Sequence | To Noise | MFI of OFF) |
| pIZ343 | Notch 1 | RKRRR (SEQ ID NO: 207) | 20.55472264 | 889.5 |
| pIZ361 | Notch 2 | KRKRKH (SEQ ID NO: 172) | 5.236923077 | 1259 |
| pIZ364 | Notch 4 | RRRREH (SEQ ID NO: 174) | 10.30799476 | 1020 |
| pSTS205 | CSF1R | KYKQKPK (SEQ ID NO: 104) | 3.093253968 | 801 |
| pSTS206 | CXCL16 | KRRR (SEQ ID NO: 105) | 14.3580786 | 1286.5 |
| pSTS208 | DAG1 | RKKRKGK (SEQ ID NO: 107) | 2.823255814 | 2519 |
| pSTS213 | GHR | KQQRIK (SEQ ID NO: 112) | 2.216494845 | 141 |
| pSTS224 | PTPRF | KRKRTH (SEQ ID NO: 123) | 10.48407643 | 1178 |
| pSTS231 | AGER | RRQRR (SEQ ID NO: 130) | 11.92390524 | 1132.5 |
| pSTS247 | KL | KKGRRSYK (SEQ ID NO: 146) | 3.723350254 | 880 |
| pSTS254 | NRG1 | KTKKQRKKLHDRLR (SEQ ID NO: 153) | 9.416666667 | 806.5 |
| pSTS255 | LRP1B | KRKRRTKTIRR (SEQ ID NO: 154) | 2.315500686 | 1014 |
| pSTS256 | Jag2 | RKRRKERERSRLPR (SEQ ID NO: 155) | 5.090909091 | 1323 |
| pSTS260 | EPCAM | RKKRMAKYEK (SEQ ID NO: 159) | 1.640226629 | 864 |
| pSTS262 | KCNE3 | RSRKVDKR (SEQ ID NO: 161) | 9.779546846 | 976 |
| pSTS263 | CDH2 | KRRDKERQAK (SEQ ID NO: 162) | 2.262402089 | 1134 |
| pSTS264 | NRG2 | KTKKQRKQMHNHLR (SEQ ID NO: 163) | 8.322951605 | 1259 |
| pSTS265 | PTPRK | KKSKLAKKRK (SEQ ID NO: 164) | 2.375 | 1248 |
| pSTS266 | BTC | HPLRKRRKRKKK (SEQ ID NO: 165) | 1.232854864 | 480 |
| pSTS267 | EPHA3 | RRRSKYSKAK (SEQ ID NO: 166) | 2.969369369 | 1289.5 |
| pSTS268 | IL1R2 | HRRCKHRTGK (SEQ ID NO: 167) | 2.413043478 | 980 |
| pSTS272 | PTPRM | KKRKLAKKRK (SEQ ID NO: 171) | 2.305630027 | 1317 |
| pSTS274 | Notch3 | RRKREH (SEQ ID NO: 173) | 12.84705882 | 1191 |
| pIZ060 | Reporter Alone | N/A | 0.408 | 114 |

### EXAMPLE 11

This Example describes Table 4 of activation characteristics of HingeNotch STS variants with T cell stimulation.

Primary human CD4+ T-cells were activated with anti-CD3/anti-CD28 Dynabeads (Gibco) and transduced with two lentiviral constructs, one expressing a hinge receptor, and the other a transcriptional reporter with constitutive eGFP+ anti-ALPPL2 CAR expression. Cells containing both constructs were sorted for Day 5 post initial T-cell stimulation and expanded further for activation testing. For testing, T-cells expressing receptors were co-cultured with K562 cells or CD19+ K562 cells. BFP reporter gene was subsequently measured using a Fortessa X-50 (BD Biosciences). Signal to noise ratios from the MFIs of BFP+cells under CD19+ K562 versus K562 conditions are plotted against the delta change in MFI in the two conditions. Activation was examined as in Example 19 but with additional ALPPL2+ K562 co-incubation for CAR activation.

TABLE 4
This table provides data with stimulation of co-expressed anti-ALPPL2
CAR with ALPPL2+ K562. “Reporter alone” represents a reporter plasmid
and was expressed in all samples.

| | | | | Average |
|---|---|---|---|---|
| | Receptor | | | MFI |
| | from which | | Average | Difference |
| Plasmid | STS is | | Signal | (MFI of ON |
| ID | derived | STS Sequence | To Noise | -MFI of OFF) |
| pIZ343 | Notch 1 | RKRRR (SEQ ID NO: 207) | 115.1879699 | 1639.5 |
| pIZ361 | Notch 2 | KRKRKH (SEQ ID NO: 172) | 15.51369863 | 4189 |
| pIZ364 | Notch 4 | RRRREH (SEQ ID NO: 174) | 24.14523449 | 1483 |
| pSTS205 | CSF1R | KYKQKPK (SEQ ID NO: 104) | 5.44982699 | 1512 |
| pSTS206 | CXCL16 | KRRR (SEQ ID NO: 105) | 27.93162393 | 1687.5 |
| pSTS208 | DAG1 | RKKRKGK (SEQ ID NO: 107) | 7.403225806 | 4639 |
| pSTS213 | GHR | KQQRIK (SEQ ID NO: 112) | 2.673553719 | −51.5 |
| pSTS224 | PTPRF | KRKRTH (SEQ ID NO: 123) | 50.78034682 | 2574.5 |
| pSTS231 | AGER | RRQRR (SEQ ID NO: 130) | 105.6603774 | 1792 |
| pSTS247 | KL | KKGRRSYK (SEQ ID NO: 146) | 6.534357661 | 1567.5 |
| pSTS254 | NRG1 | KTKKQRKKLHDRLR (SEQ ID NO: 153) | 24.99036609 | 637 |
| pSTS255 | LRP1B | KRKRRTKTIRR (SEQ ID NO: 154) | 10.30285381 | 2558 |
| pSTS256 | Jag2 | RKRRKERERSRLPR (SEQ ID NO: 155) | 21.38095238 | 3320.5 |
| pSTS260 | EPCAM | RKKRMAKYEK (SEQ ID NO: 159) | 2.48189415 | 2372.5 |
| pSTS262 | KCNE3 | RSRKVDKR (SEQ ID NO: 161) | 62.54752852 | 1900.5 |
| pSTS263 | CDH2 | KRRDKERQAK (SEQ ID NO: 162) | 4.24047619 | 2834 |
| pSTS264 | NRG2 | KTKKQRKQMHNHLR (SEQ ID NO: 163) | 15.82959641 | 2201 |
| pSTS265 | PTPRK | KKSKLAKKRK (SEQ ID NO: 164) | 6.761506276 | 2514.5 |
| pSTS266 | BTC | HPLRKRRKRKKK (SEQ ID NO: 165) | 1.713168188 | 423 |
| pSTS267 | EPHA3 | RRRSKYSKAK (SEQ ID NO: 166) | 6.307971014 | 2274 |
| pSTS268 | IL1R2 | HRRCKHRTGK (SEQ ID NO: 167) | 4.775 | 2027 |
| pSTS272 | PTPRM | KKRKLAKKRK (SEQ ID NO: 171) | 9.289501591 | 2517.5 |
| pSTS274 | Notch3 | RRKREH (SEQ ID NO: 173) | 29.22939068 | 1987.5 |
| pIZ060 | Reporter Alone | N/A | 1.719298246 | 422.5 |

### EXAMPLE 12

This Example describes mutational analysis for the transmembrane domain (TMD) and the STS domain in Hinge-Notch constructs.

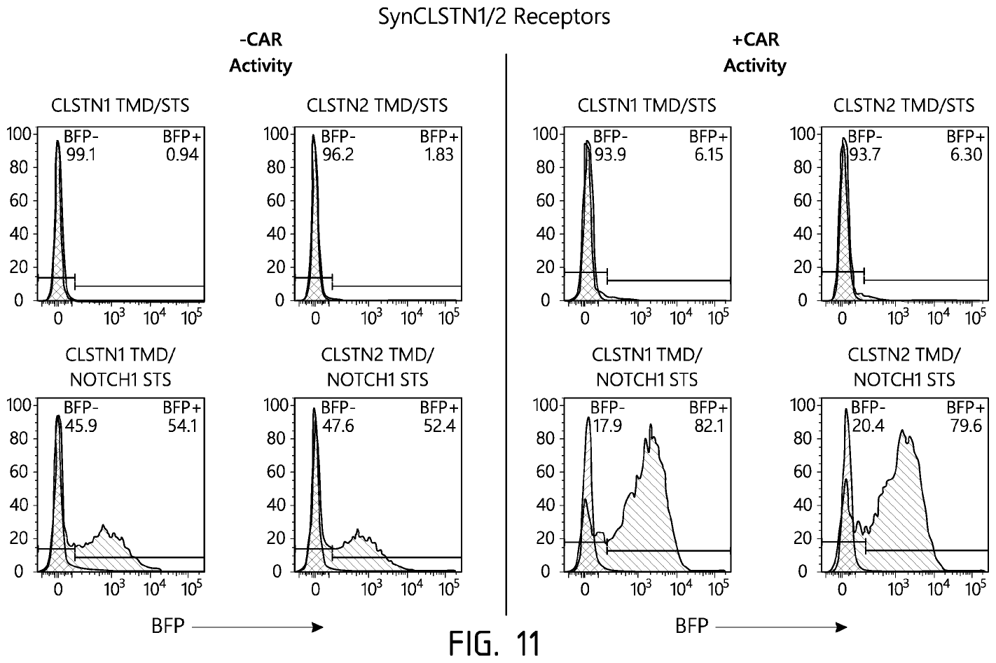

Four types of exemplary Hinge Notch receptors (SEQ ID NOS: 237-240) were using in this Example, all of which including an anti-CD19 scFv domain, a truncated CD8 Hinge domain, and a Gal4VP64 domain. For choices of STS and TMD domains, the four constructs comprise: CLSTN1 TMD and CLSTN1 STS (SEQ ID NO: 237), CLSTN2 TMD and CLSTN2 STS (SEQ ID NO: 238), CLSTN1 TMD and Notch1 STS (SEQ ID NO: 239), CLSTN2 TMD and Notch1 STS (SEQ ID NO: 240). Primary human CD4+ T-cells were activated with anti-CD3/anti-CD28 Dynabeads (Gibco) and transduced with two lentiviral constructs, one expressing a hinge receptor with TMD/STS combination as indicated, and the other a transcriptional reporter with constitutively expressed anti-ALPPL2 CAR. Cells containing both constructs were sorted for on Day 5 post initial T-cell stimulation and expanded further for activation testing. As shown in FIG. 11, 1×10⁵ double positive T-cells expressing receptors were co-cultured with: 1×10⁵ K562 cells (“-CAR” panels, blue), or 1×10⁵ CD19+K562 cells (“-CAR” panels, red). Similarly, 1×10⁵ double positive T-cells expressing receptors were tested in the presence of CAR activity by co-culture with 1×10⁵ ALPPL2+ K562 cells (“+CAR” panels, blue), or 1×10⁵ ALPPL2+ CD19+ K562 cells (“+CAR” panels, red). Transcriptional activation of an inducible BFP reporter gene was subsequently measured using a Fortessa X-50 (BD Biosciences).

### EXAMPLE 13

This Example describes experiments performed to demonstrate controlled IL-2 production by T cells engineered Hinge-Notch STS variants.

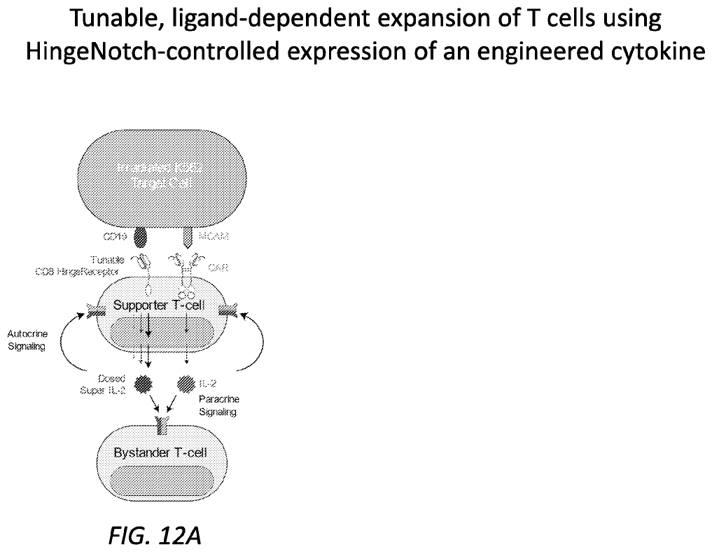

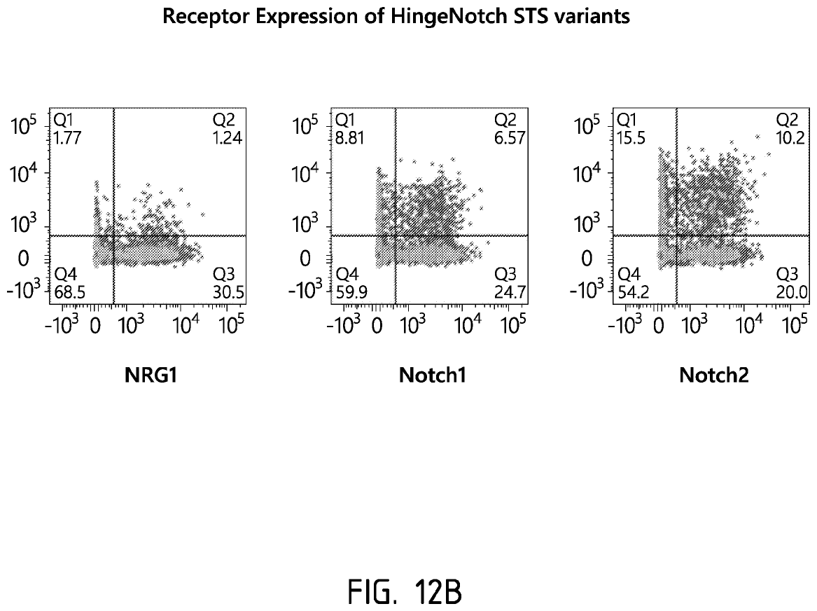

FIG. 12A shows a diagram of T cells engineered with Hinge-Notch STS variants to provide ligand-triggered secretion of an engineered cytokine for autocrine and paracrine expansion of T cells. Expression profile of anti-CD19 Hinge-Notch receptors with the indicated STS modifications are shown in FIG. 12B. Primary human T-cells were activated with anti-CD3/anti-CD28 Dynabeads (Gibco) and transduced with two lentiviral constructs, one expressing a CAR against the MCAM antigen, and one expressing a Hinge-Notch receptor with inducible super-IL2 under Gal4-UAS control. Cells containing both constructs were sorted on Day 5 post initial T-cell stimulation and expanded further for activation testing. Receptor expression was determined by anti-myc-tag staining (y-axis).

### EXAMPLE 14

This Example describes experiments performed to demonstrate that ligand-triggered expression of super-IL2 improves cell viability of CAR-T cells.

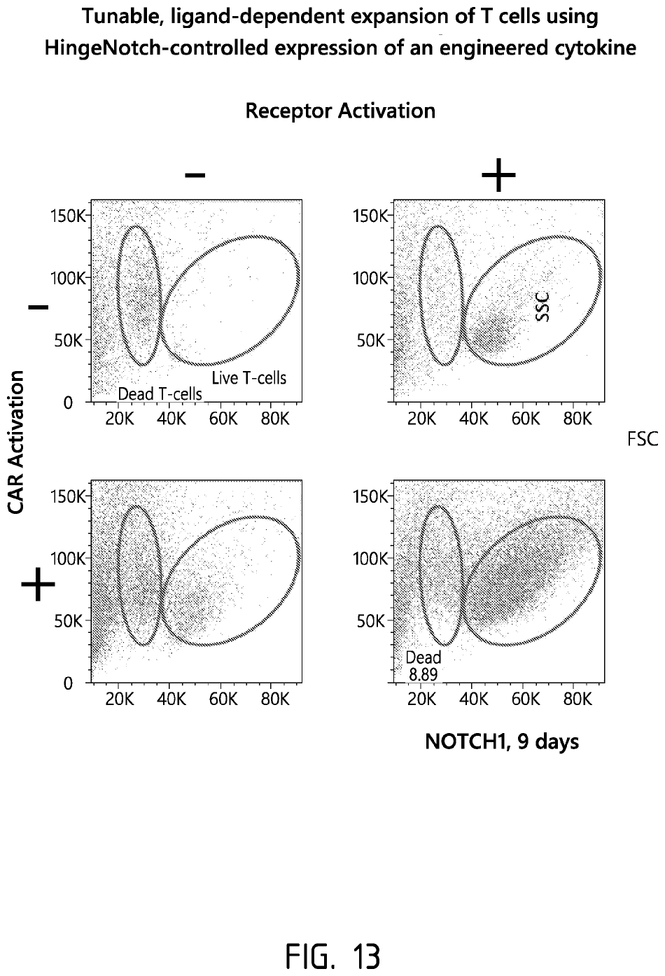

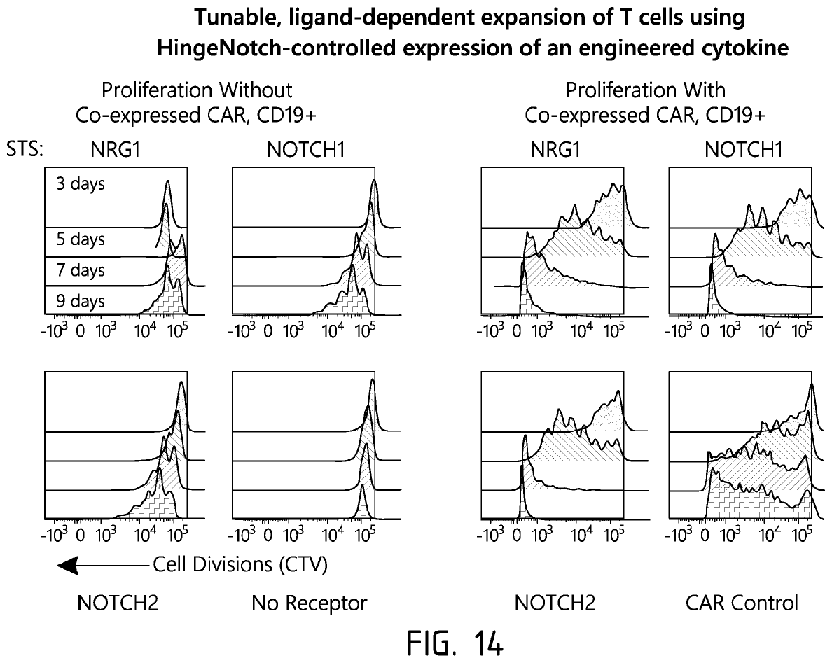

1×10⁵ double positive T-cells expressing anti-CD19 Hinge-Notch Notch1 STS receptors were co-cultured in media without IL-2, with no K562 cells (top left), with CD19+ K562 cells to trigger Hinge-Notch (top right), with MCAM+ K562 cells to trigger CAR activation (bottom left) or with MCAM+ and CD19+ K562 cells to trigger activation of both receptors (bottom right) (FIG. 13). After 9 days, the proportion of live T cells by forward and side-scatter measurements using a Fortessa X-50 (BD Biosciences) was assessed. Co- activation of both receptors resulted in the most viable cells, followed by Hinge-Notch activation (and subsequent super-IL2 induction), CAR activation alone, and no activation of either receptor.

### EXAMPLE 15

This Example describes experiments performed to demonstrate tunable proliferation of T cells with STS-variants of Hinge-Notch.

Primary human T-cells were activated with anti-CD3/anti-CD28 Dynabeads (Gibco) and transduced with two lentiviral constructs, one expressing a CAR against the MCAM antigen, and one expressing a Hinge-Notch receptor with inducible super-IL2 under Gal4-UAS control (the right four panels of FIG. 14). Hinge-Notch receptors containing 3 different STS variants (NRG1, Notch1, and Notch2) were tested against a no Hinge-Notch control. Similarly, primary human T-cells were generated without CAR expression (left panels of FIG. 14). T cells were stained with CellTrace Violet (Invitrogen) according to manufacturer's protocols, co-incubated with CD19+ K562 target cells in media without IL-2 and measured using a Fortessa X-50 (BD Biosciences) at the indicated timepoints to assess proliferation by CTV signal decay.

### EXAMPLE 16

This Example describes experiments performed to demonstrate tunable secretion of super-IL2 with STS-variants of Hinge-Notch.

Primary human T-cells were activated with anti-CD3/anti-CD28 Dynabeads (Gibco) and transduced with a lentiviral construct Hinge-Notch receptor with inducible super-IL2 under Gal4-UAS control (FIG. 15A). Hinge-Notch receptors containing 3 different STS variants (NRG1, Notch1, and Notch2) were tested against a no HingeNotch control. T cells were co-incubated with MCAM+ CD19+ K562 cells in media lacking IL-2, and at the indicated timepoints, supernatant IL-2 was measured using the Instant ELISA Kit (Invitrogen) according to manufacturer's protocols with a microplate reader (Tecan). Red dotted line indicates a standard concentration of IL-2 used for culturing T cells. Graded secretion of super-IL2 was achieved by activation of STS-tuned HingeNotch receptors.

For FIG. 15B, primary human T-cells were generated with an additional lentiviral vector expressing a CAR against MCAM. Enhanced uptake of IL-2 by CAR-expressing cells resulted in loss of supernatant IL2 in CAR-only and NRG1-STS Hinge-Notch T cells. In contrast, greater induction of super-IL2 by Notch1 and Notch2-STS based receptors initially outpaces this uptake, before proliferation and K562 elimination reduces supernatant levels.

### EXAMPLE 17

This Example describes experiments performed to demonstrate tunable secretion of super-IL2 with STS-variants of Hinge-Notch enhances proliferation of bystander T cells.

Primary human T-cells were activated with anti-CD3/anti-CD28 Dynabeads (Gibco) and transduced with a lentiviral construct comprising a Hinge-Notch receptor with inducible super-IL2 under Gal4-UAS control (right panels of FIG. 16). HingeNotch receptors containing 3 different STS variants (NRG1, Notch1, and Notch2) were tested against a no HingeNotch control. HingeNotch T cells were co-incubated with “bystander” T cells stained with CellTrace Far Red (Invitrogen) expressing a CAR against MCAM (left panel of FIG. 16) or with no CAR (right panel of FIG. 16). T cells were co-incubated with MCAM+ CD19+ K562 cells in media lacking IL-2, and proliferation of the bystander T cells were assessed by measuring signal decay on a Fortessa X-50 (BD Biosciences). For bystander T cells with and without CAR expression, proliferation was enhanced in graded fashion by STS variants of Hinge-Notch-activated T cells.

### EXAMPLE 18

This Example describes experiments performed to test single lentiviral vector constructs containing Hinge-Notch receptors CAR circuits.

Primary human T-cells were activated with anti-CD3/anti-CD28 Dynabeads (Gibco) and transduced with a single lentiviral construct containing constitutively expressed Hinge- Notch receptors with an inducible anti-MCAM CAR cassette under Gal4-UAS control. Cells were sorted for Hinge-Notch receptor expression via myc-tag on Day 5 post initial T-cell stimulation and expanded further for activation testing. Three STS-variants were tested as indicated, with constitutively expressed CAR used as a control (FIG. 17). For testing, 1×10⁵ T cells expressing anti-CD19 receptors were co-cultured with: no additions (upper trace), 5×10⁵ K562 cells (middle trace), or 5×10⁴ CD19+ K562 cells (lower trace). Transcriptional activation of the inducible CAR was subsequently measured by a GFP tag using a Fortessa X-50 (BD Biosciences).

### EXAMPLE 19

This Example describes experiments performed to demonstrate specific dual antigen target cell killing by T cells engineered with a single lentivector containing a HingeNotch CAR circuit.

Primary human T-cells were activated with anti-CD3/anti-CD28 Dynabeads (Gibco) and transduced with a single lentiviral construct containing constitutively expressed HingeNotch -receptors with an inducible anti-MCAM CAR cassette under Gal4-UAS control. Cells were sorted for Hinge-Notch receptor expression via myc-tag on Day 5 post initial T-cell stimulation and expanded further for activation testing. Three STS-variants were tested as indicated, with constitutively expressed CAR used as a control. For testing, 1×10⁵ T-cells expressing anti-CD19 receptors were co-cultured with 5×10⁵ MCAM+ K562 cells or 5×10⁴ MCAM+ CD19+ K562 cells. Target cell killing was assessed by forward/side-scatter of the K562 population using a Fortessa X-50 (BD Biosciences). As shown in FIG. 18, Hinge-Notch circuits effectively and specifically clear target cells containing both MCAM+ and CD19+antigens.

### EXAMPLE 20

This Example describes experiments performed for testing single lentiviral vector constructs containing Hinge-Notch receptors for control of T cell activation and exhaustion.

Primary human T-cells were activated with anti-CD3/anti-CD28 Dynabeads (Gibco) and transduced with a single lentiviral construct containing constitutively expressed Hinge-Notch receptors with an inducible anti-MCAM CAR cassette under Gal4-UAS control. Cells were sorted for Hinge-Notch receptor expression via myc-tag on Day 5 post initial T-cell stimulation and expanded further for activation testing. Three STS-variants were tested as indicated, with constitutively expressed CAR used as a control. For testing, 1×10⁵ T-cells expressing anti-CD19 receptors were co-cultured with 5×10⁴ CD19+ K562 cells. Transcriptional activation of the inducible CAR was subsequently measured by a GFP tag using a Fortessa X-50 (BD Biosciences) (the left most panel of FIG. 19). T cell activation and exhaustion were measure by expression of CD25 and CD39, respectively (FIG. 19).

### EXAMPLE 21

This Example describes experiments performed for in vivo testing of Hinge-Notch-to-CAR circuits.

As shown in FIG. 20, for unilateral tumors, NOD.Cg-Prkd^{scid}Il2rg^{tm1Wjl}/SzJ (NSG) mice were implanted with 1×10⁶ K562-BCMA/CD19 tumor cells subcutaneously on the left flank. For contralateral tumors, NSG mice were implanted with 1×10⁶ K562-BCMA/CD19 tumor cells on the left flank and with 1×10⁶ K562-CD19 tumor cells on the right flank. Four days after tumor implantation, 2.5×10⁶ engineered primary human CD4+ and CD8+ T cells (total of 5×10⁶ T cells) were infused i.v. through tail vein injection. Tumor size was monitored via caliper 2-3 per week and mice were determined to have reached endpoint when tumors measured ≥20 mm For immunophenotypic analysis, tumors and spleens were harvested 10 days post T cell implantation. Tumors were manually minced and digested in RPMI-1640 with 4 mg/ml Collagenase IV (Worthington Biochemical Corporation) and 0.1 mg/ml DNase I (MilliporeSigma) at 37° C. for 30 min and spleens were manually dissociated and subjected to red blood cell lysis (ACK; KD medical). The following antibodies were used: anti-CD45 (2D1, 368516, Biolegend), anti-CD3 (UCHT1, 300464, Biolegend), anti-CD4 (SK3, 563552, BD biosciences), and anti-CD8 (RPA-T8, 563823, BD biosciences). Dead cells were excluded with Draq7 (Abcam). Samples were analyzed using FACSymphony X50 SORP (BD Biosciences) and data was analyzed using FlowJo software (BD Biosciences).

### EXAMPLE 22

This Example describes the results of experiments as described herein for some exemplary Notch receptors.

TABLE 5

| | | Full-length | |
|---|---|---|---|
| Construct ID | Receptor Description | SEQ ID NO | Experiment Result for Activity |
| pIZ341 | anti-CD19scFv- | 219 | Demonstrated that a full CD8 Hinge is |
| | CD8Hinge-Notch1TMD- | | sufficient as a ligand-sensitive domain. |
| | Notch1 STS-Gal4VP64 | | |
| pIZ343 | anti-CD19scFv- | 204 | Demonstrated that a truncated CD8 Hinge is |
| | CD8Hinge2-Notch1TMD- | | sufficient as a ligand-sensitive domain, better |
| | Notch1 STS -Gal4VP64 | | controlled than pIZ341 or pIZ342. |
| pIZ358 | anti-CD19scFv- | 220 | Demonstrated that other hinge domains |
| | CD28Hinge-Notch1TMD- | | besides CD8 Hinge can be used as ligand- |
| | Notch1 STS -Gal4VP64 | | sensitive domains, activates about as well as a |
| | | | truncated CD8 Hinge. |
| pIZ359 | anti-CD19scFv- | 221 | Demonstrated that other hinge domains |
| | IgG4Hinge-Notch1TMD- | | besides CD8 Hinge can be used as ligand- |
| | Notch1 STS-Gal4VP64 | | sensitive domains. |
| pIZ360 | anti-CD19scFv-OX40- | 222 | Demonstrates that other hinge domains |
| | Notch1TMD-Notch1 STS - | | besides CD8 Hinge can be used as ligand- |
| | Gal4VP64 | | sensitive domains. |
| pIZ361 | anti-CD19scFv- | 205 | Demonstrates that Notch2 STS improves |
| | CD8Hinge2-Notch1TMD- | | signal of receptors that do not contain Notch- |
| | Notch2STS-Gal4VP64 | | based ECDs. |
| pIZ343FYIA | anti-ALPPL2scFv- | 223 | Activated against M28, ALPPL2+ K562. |
| | CD8Hinge2-Notch1TMD- | | |
| | Notch1 STS -Gal4VP64 | | |
| pIZ343eGFP | eGFP-CD8Hinge2- | 224 | Activated against LaG17+ K562. |
| | Notch1TMD-Notch1 STS - | | |
| | Gal4VP64 | | |
| pIZ342 | anti-CD19scFv- | 225 | Demonstrated that a truncated CD8 Hinge is |
| | CD8Hinge1-Notch1TMD- | | sufficient as a ligand-sensitive domain, better |
| | Notch1 STS-Gal4VP64 | | controlled than pIZ341. |
| pIZ362 | anti-CD19scFV- | 226 | Demonstrated that a truncated CD8 Hinge is |
| | CD8Hinge3-Notch1TMD- | | sufficient as a ligand-sensitive domain, |
| | Notch1STS-Gal4VP64 | | weaker than pIZ343. |
| pIZ363 | anti-CD19scFV- | 227 | Demonstrated that a truncated CD8 Hinge is |
| | CD8Hinge4-Notch1TMD- | | sufficient as a ligand-sensitive domain, |
| | Notch1STS-Gal4VP64 | | weaker than pIZ343. |
| pIZ361FYIA | anti-ALPPL2scFv- | 228 | Activated against M28, ALPPL2+ K562. |
| | CD8Hinge2-Notch1TMD- | | |
| | Notch2STS-Gal4VP64 | | |
| pIZ343BCMA | anti-BCMAscFV- | 229 | Activated against BCMA+ K562. |
| | CD8Hinge2-Notch1TMD- | | |
| | Notch1 STS-Gal4VP64 | | |
| pIZ361BCMA | anti-BCMAscFV- | 230 | Activated against BCMA+ K562. |
| | CD8Hinge2-Notch1TMD- | | |
| | Notch2STS-Gal4VP64 | | |
| pIZ343(4D5-8) | anti-Her2scFV_4D5-8- | 231 | Activated against Her2+ SKBR3. |
| | CD8Hinge2-Notch1TMD- | | |
| | Notch1 STS-Gal4VP64 | | |
| pIZ361(4D5-8) | anti-Her2scFV_4D5-8- | 232 | Activated against Her2+ SKBR3. |
| | CD8Hinge2-Notch1TMD- | | |
| | Notch2STS-Gal4VP64 | | |
| pIZ343(4D5-7) | anti-Her2scFV_4D5-7- | 233 | Activated against Her2+ SKBR3. |
| | CD8Hinge2-Notch1TMD- | | |
| | Gal4VP64 | | |
| pIZ361(4D5-7) | anti-Her2scFV_4D5-7- | 234 | Activated against Her2+ SKBR3. |
| | CD8Hinge2-Notch1TMD- | | |
| | Notch2STS-Gal4VP64 | | |
| pRay068A | anti-BCMA_FHVH33- | 235 | Activated against BCMA+ K562. |
| | CD8Hinge2-Notch1TMD- | | |
| | Notch2STS-Gal4VP64 | | |
| pRay068B | anti-BCMA_FHVH33- | 236 | Activated against BCMA+ K562. |
| | CD8Hinge5-Notch1TMD- | | |
| | Notch2STS-Gal4VP64 | | |
| pIZ370 | anti-CD19scFv- | 237 | Activated poorly against CD19+ K562. |
| | CD8Hinge2- | | |
| | CLSTN1TMD- | | |
| | CLSTN1STS-Gal4VP64 | | |
| pIZ371 | antiCD19scFv- | 238 | Activated poorly against CD19+ K562. |
| | CD8Hinge2- | | |
| | CLSTN2TMD- | | |
| | CLSTN2STS-Gal4VP64 | | |
| pTMD201 | antiCD19scFv- | 239 | Activated well against CD19+ K562. |
| | CD8Hinge2- | | |
| | CLSTN1TMD- | | |
| | Notch1STS-Gal4VP64 | | |
| pTMD202 | antiCD19scFv- | 240 | Activated well against CD19+ K562. |
| | CD8Hinge2- | | |
| | CLSTN2TMD- | | |
| | Notch1STS-Gal4VP64 | | |

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

SEQUENCE LISTING

| SEQ | | |
|---|---|---|
| ID | Sequence | Description |
| 1 | MALPVTALLLPLALLLHAARPEQKLISEEDLDIQMTQTTSSLSASLGDRVT | pSTS001 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with APLP1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRKKMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 2 | MALPVTALLLPLALLLHAARPEQKLISEEDLDIQMTQTTSSLSASLGDRVT | pSTS002 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with APLP2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 3 | MALPVTALLLPLALLLHAARPEQKLISEEDLDIQMTQTTSSLSASLGDRVT | pSTS003 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with APP STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKKKMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 4 | MALPVTALLLPLALLLHAARPEQKLISEEDLDIQMTQTTSSLSASLGDRVT | pSTS004 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with TGBR3 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSHMKLLSSIEQACDICRLKKLKCSKEKPKCAKCLKNNWE | |
| | CRYSPKTKRSPLTRAHLTEVESRLERLEQLFLLIFPREDLDMILKMDSLQD | |
| | IKALLTGLFVQDNVNKDAVTDRLASVETDMPLTLRQHRISATSSSEESSN | |
| | KGQRQLTVSAAAGGSGGSGGSDALDDFDLDMLGSDALDDFDLDMLGSD | |
| | ALDDFDLDMLGSDALDDFDLDMLGS | |
| 5 | MALPVTALLLPLALLLHAARPEQKLISEEDLDIQMTQTTSSLSASLGDRVT | pSTS005 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CSF1R |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKYKQKPKMKLLSSIEQACDICRLKKLKCSKEKPKCAKCL | |
| | KNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 6 | MALPVTALLLPLALLLHAARPEQKLISEEDLDIQMTQTTSSLSASLGDRVT | pSTS006 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CXCL16 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRRRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 7 | MALPVTALLLPLALLLHAARPEQKLISEEDLDIQMTQTTSSLSASLGDRVT | pSTS007 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CX3CL1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKMKLLSSIEQACDICRLKKLKCSKEKPKCAKCLKNNW | |
| | ECRYSPKTKRSPLTRAHLTEVESRLERLEQLFLLIFPREDLDMILKMDSLQ | |
| | DIKALLTGLFVQDNVNKDAVTDRLASVETDMPLTLRQHRISATSSSEESS | |
| | NKGQRQLTVSAAAGGSGGSGGSDALDDFDLDMLGSDALDDFDLDMLGS | |
| | DALDDFDLDMLGSDALDDFDLDMLGS | |
| 8 | MALPVTALLLPLALLLHAARPEQKLISEEDLDIQMTQTTSSLSASLGDRVT | pSTS008 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with DAGI |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKKRKGKMKLLSSIEQACDICRLKKLKCSKEKPKCAKCL | |
| | KNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 9 | MALPVTALLLPLALLLHAARPEQKLISEEDLDIQMTQTTSSLSASLGDRVT | pSTS009 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with DCC STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRMKLLSSIEQACDICRLKKLKCSKEKPKCAKCLKNNW | |
| | ECRYSPKTKRSPLTRAHLTEVESRLERLEQLFLLIFPREDLDMILKMDSLQ | |
| | DIKALLTGLFVQDNVNKDAVTDRLASVETDMPLTLRQHRISATSSSEESS | |
| | NKGQRQLTVSAAAGGSGGSGGSDALDDFDLDMLGSDALDDFDLDMLGS | |
| | DALDDFDLDMLGSDALDDFDLDMLGS | |
| 10 | MALPVTALLLPLALLLHAARPEQKLISEEDLDIQMTQTTSSLSASLGDRVT | pSTS010 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with DNER |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRISRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 11 | MALPVTALLLPLALLLHAARPEQKLISEEDLDIQMTQTTSSLSASLGDRVT | pSTS011 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with DSG2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKCGKGAKMKLLSSIEQACDICRLKKLKCSKEKPKCAKCL | |
| | KNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 12 | MALPVTALLLPLALLLHAARPEQKLISEEDLDIQMTQTTSSLSASLGDRVT | pSTS012 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CDH1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 13 | MALPVTALLLPLALLLHAARPEQKLISEEDLDIQMTQTTSSLSASLGDRVT | pSTS013 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with GHR STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKQQRIKMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 14 | MALPVTALLLPLALLLHAARPEQKLISEEDLDIQMTQTTSSLSASLGDRVT | pSTS014 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with HLA-A |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRKMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 15 | MALPVTALLLPLALLLHAARPEQKLISEEDLDIQMTQTTSSLSASLGDRVT | pSTS015 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with IFNAR2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKMKLLSSIEQACDICRLKKLKCSKEKPKCAKCLKNNWE | |
| | CRYSPKTKRSPLTRAHLTEVESRLERLEQLFLLIFPREDLDMILKMDSLQD | |
| | IKALLTGLFVQDNVNKDAVTDRLASVETDMPLTLRQHRISATSSSEESSN | |
| | KGQRQLTVSAAAGGSGGSGGSDALDDFDLDMLGSDALDDFDLDMLGSD | |
| | ALDDFDLDMLGSDALDDFDLDMLGS | |
| 16 | MALPVTALLLPLALLLHAARPEQKLISEEDLDIQMTQTTSSLSASLGDRVT | pSTS016 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with IGFIR |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSHRKRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 17 | MALPVTALLLPLALLLHAARPEQKLISEEDLDIQMTQTTSSLSASLGDRVT | pSTS017 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with IL1R1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKIFKMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 18 | MALPVTALLLPLALLLHAARPEQKLISEEDLDIQMTQTTSSLSASLGDRVT | pSTS018 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with ERN2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRMKLLSSIEQACDICRLKKLKCSKEKPKCAKCLKNNWE | |
| | CRYSPKTKRSPLTRAHLTEVESRLERLEQLFLLIFPREDLDMILKMDSLQD | |
| | IKALLTGLFVQDNVNKDAVTDRLASVETDMPLTLRQHRISATSSSEESSN | |
| | KGQRQLTVSAAAGGSGGSGGSDALDDFDLDMLGSDALDDFDLDMLGSD | |
| | ALDDFDLDMLGSDALDDFDLDMLGS | |
| 19 | MALPVTALLLPLALLLHAARPEQKLISEEDLDIQMTQTTSSLSASLGDRVT | pSTS019 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with KCNE1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRSKKLEHMKLLSSIEQACDICRLKKLKCSKEKPKCAKCL | |
| | KNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 20 | MALPVTALLLPLALLLHAARPEQKLISEEDLDIQMTQTTSSLSASLGDRVT | pSTS020 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with KCNE2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKSKRREHMKLLSSIEQACDICRLKKLKCSKEKPKCAKCL | |
| | KNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 21 | MALPVTALLLPLALLLHAARPEQKLISEEDLDIQMTQTTSSLSASLGDRVT | pSTS021 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CHL1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRNRGGKMKLLSSIEQACDICRLKKLKCSKEKPKCAKCL | |
| | KNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 22 | MALPVTALLLPLALLLHAARPEQKLISEEDLDIQMTQTTSSLSASLGDRVT | pSTS022 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with LRP1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 23 | MALPVTALLLPLALLLHAARPEQKLISEEDLDIQMTQTTSSLSASLGDRVT | pSTS023 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with LRP2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSHYRRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 24 | MALPVTALLLPLALLLHAARPEQKLISEEDLDIQMTQTTSSLSASLGDRVT | pSTS024 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with PTPRF |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRKRTHMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 25 | MALPVTALLLPLALLLHAARPEQKLISEEDLDIQMTQTTSSLSASLGDRVT | pSTS025 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SCN1B |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKKMKLLSSIEQACDICRLKKLKCSKEKPKCAKCLKNNW | |
| | ECRYSPKTKRSPLTRAHLTEVESRLERLEQLFLLIFPREDLDMILKMDSLQ | |
| | DIKALLTGLFVQDNVNKDAVTDRLASVETDMPLTLRQHRISATSSSEESS | |
| | NKGQRQLTVSAAAGGSGGSGGSDALDDFDLDMLGSDALDDFDLDMLGS | |
| | DALDDFDLDMLGSDALDDFDLDMLGS | |
| 26 | MALPVTALLLPLALLLHAARPEQKLISEEDLDIQMTQTTSSLSASLGDRVT | pSTS026 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SCN3B |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKVSKMKLLSSIEQACDICRLKKLKCSKEKPKCAKCLKN | |
| | NWECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSEE | |
| | SSNKGQRQLTVSAAAGGSGGSGGSDALDDFDLDMLGSDALDDFDLDML | |
| | GSDALDDFDLDMLGSDALDDFDLDMLGS | |
| 27 | MALPVTALLLPLALLLHAARPEQKLISEEDLDIQMTQTTSSLSASLGDRVT | pSTS027 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with NPR3 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKKYRMKLLSSIEQACDICRLKKLKCSKEKPKCAKCLKN | |
| | NWECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSEE | |
| | SSNKGQRQLTVSAAAGGSGGSGGSDALDDFDLDMLGSDALDDFDLDML | |
| | GSDALDDFDLDMLGSDALDDFDLDMLGS | |
| 28 | MALPVTALLLPLALLLHAARPEQKLISEEDLDIQMTQTTSSLSASLGDRVT | pSTS028 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with NGFR |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRMKLLSSIEQACDICRLKKLKCSKEKPKCAKCLKNNW | |
| | ECRYSPKTKRSPLTRAHLTEVESRLERLEQLFLLIFPREDLDMILKMDSLQ | |
| | DIKALLTGLFVQDNVNKDAVTDRLASVETDMPLTLRQHRISATSSSEESS | |
| | NKGQRQLTVSAAAGGSGGSGGSDALDDFDLDMLGSDALDDFDLDMLGS | |
| | DALDDFDLDMLGSDALDDFDLDMLGS | |
| 29 | MALPVTALLLPLALLLHAARPEQKLISEEDLDIQMTQTTSSLSASLGDRVT | pSTS029 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with PLXDC2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSHHMKLLSSIEQACDICRLKKLKCSKEKPKCAKCLKNNW | |
| | ECRYSPKTKRSPLTRAHLTEVESRLERLEQLFLLIFPREDLDMILKMDSLQ | |
| | DIKALLTGLFVQDNVNKDAVTDRLASVETDMPLTLRQHRISATSSSEESS | |
| | NKGQRQLTVSAAAGGSGGSGGSDALDDFDLDMLGSDALDDFDLDMLGS | |
| | DALDDFDLDMLGSDALDDFDLDMLGS | |
| 30 | MALPVTALLLPLALLLHAARPEQKLISEEDLDIQMTQTTSSLSASLGDRVT | pSTS030 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with PAM STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRWKKSRMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 31 | MALPVTALLLPLALLLHAARPEQKLISEEDLDIQMTQTTSSLSASLGDRVT | pSTS031 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with AGER |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRQRRMKLLSSIEQACDICRLKKLKCSKEKPKCAKCLKN | |
| | NWECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSEE | |
| | SSNKGQRQLTVSAAAGGSGGSGGSDALDDFDLDMLGSDALDDFDLDML | |
| | GSDALDDFDLDMLGSDALDDFDLDMLGS | |
| 32 | MALPVTALLLPLALLLHAARPEQKLISEEDLDIQMTQTTSSLSASLGDRVT | pSTS032 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with ROBO1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRHRKKRMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 33 | MALPVTALLLPLALLLHAARPEQKLISEEDLDIQMTQTTSSLSASLGDRVT | pSTS033 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SORCS3 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRKMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 34 | MALPVTALLLPLALLLHAARPEQKLISEEDLDIQMTQTTSSLSASLGDRVT | pSTS034 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SORCS1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKFKRRMKLLSSIEQACDICRLKKLKCSKEKPKCAKCLKN | |
| | NWECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSEE | |
| | SSNKGQRQLTVSAAAGGSGGSGGSDALDDFDLDMLGSDALDDFDLDML | |
| | GSDALDDFDLDMLGSDALDDFDLDMLGS | |
| 35 | MALPVTALLLPLALLLHAARPEQKLISEEDLDIQMTQTTSSLSASLGDRVT | pSTS035 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SORL1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKHRRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 36 | MALPVTALLLPLALLLHAARPEQKLISEEDLDIQMTQTTSSLSASLGDRVT | pSTS036 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SDC1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRMKKKMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 37 | MALPVTALLLPLALLLHAARPEQKLISEEDLDIQMTQTTSSLSASLGDRVT | pSTS037 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SDC2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRMRKKMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 38 | MALPVTALLLPLALLLHAARPEQKLISEEDLDIQMTQTTSSLSASLGDRVT | pSTS038 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SPN STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRRQKRRMKLLSSIEQACDICRLKKLKCSKEKPKCAKCL | |
| | KNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 39 | MALPVTALLLPLALLLHAARPEQKLISEEDLDIQMTQTTSSLSASLGDRVT | pSTS039 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with TYR STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRHKRKMKLLSSIEQACDICRLKKLKCSKEKPKCAKCLKN | |
| | NWECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSEE | |
| | SSNKGQRQLTVSAAAGGSGGSGGSDALDDFDLDMLGSDALDDFDLDML | |
| | GSDALDDFDLDMLGSDALDDFDLDMLGS | |
| 40 | MALPVTALLLPLALLLHAARPEQKLISEEDLDIQMTQTTSSLSASLGDRVT | pSTS040 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with TYRP1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRARRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 41 | MALPVTALLLPLALLLHAARPEQKLISEEDLDIQMTQTTSSLSASLGDRVT | pSTS041 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with DCT STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRLRKMKLLSSIEQACDICRLKKLKCSKEKPKCAKCLKN | |
| | NWECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSEE | |
| | SSNKGQRQLTVSAAAGGSGGSGGSDALDDFDLDMLGSDALDDFDLDML | |
| | GSDALDDFDLDMLGSDALDDFDLDMLGS | |
| 42 | MALPVTALLLPLALLLHAARPEQKLISEEDLDIQMTQTTSSLSASLGDRVT | pSTS042 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with VASN |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRGRMKLLSSIEQACDICRLKKLKCSKEKPKCAKCLKNN | |
| | WECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEES | |
| | SNKGQRQLTVSAAAGGSGGSGGSDALDDFDLDMLGSDALDDFDLDMLG | |
| | SDALDDFDLDMLGSDALDDFDLDMLGS | |
| 43 | MALPVTALLLPLALLLHAARPEQKLISEEDLDIQMTQTTSSLSASLGDRVT | pSTS043 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with FLT1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKMKRMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 44 | MALPVTALLLPLALLLHAARPEQKLISEEDLDIQMTQTTSSLSASLGDRVT | pSTS044 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CDH5 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRRLRKQARAHGKMKLLSSIEQACDICRLKKLKCSKEKP | |
| | KCAKCLKNNWECRYSPKTKRSPLTRAHLTEVESRLERLEQLFLLIFPRED | |
| | LDMILKMDSLQDIKALLTGLFVQDNVNKDAVTDRLASVETDMPLTLRQH | |
| | RISATSSSEESSNKGQRQLTVSAAAGGSGGSGGSDALDDFDLDMLGSDAL | |
| | DDFDLDMLGSDALDDFDLDMLGSDALDDFDLDMLGS | |
| 45 | MALPVTALLLPLALLLHAARPEQKLISEEDLDIQMTQTTSSLSASLGDRVT | pSTS045 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with PKHD1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRSKSRKTKMKLLSSIEQACDICRLKKLKCSKEKPKCAK | |
| | CLKNNWECRYSPKTKRSPLTRAHLTEVESRLERLEQLFLLIFPREDLDMIL | |
| | KMDSLQDIKALLTGLFVQDNVNKDAVTDRLASVETDMPLTLRQHRISAT | |
| | SSSEESSNKGQRQLTVSAAAGGSGGSGGSDALDDFDLDMLGSDALDDFD | |
| | LDMLGSDALDDFDLDMLGSDALDDFDLDMLGS | |
| 46 | MALPVTALLLPLALLLHAARPEQKLISEEDLDIQMTQTTSSLSASLGDRVT | pSTS046 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with NECTIN1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRRRHTFKMKLLSSIEQACDICRLKKLKCSKEKPKCAKC | |
| | LKNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 47 | MALPVTALLLPLALLLHAARPEQKLISEEDLDIQMTQTTSSLSASLGDRVT | pSTS047 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with KL STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKKGRRSYKMKLLSSIEQACDICRLKKLKCSKEKPKCAKC | |
| | LKNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 48 | MALPVTALLLPLALLLHAARPEQKLISEEDLDIQMTQTTSSLSASLGDRVT | pSTS048 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with IL6R STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRFKKTWKLRALKEGKMKLLSSIEQACDICRLKKLKCSKE | |
| | KPKCAKCLKNNWECRYSPKTKRSPLTRAHLTEVESRLERLEQLFLLIFPRE | |
| | DLDMILKMDSLQDIKALLTGLFVQDNVNKDAVTDRLASVETDMPLTLRQ | |
| | HRISATSSSEESSNKGQRQLTVSAAAGGSGGSGGSDALDDFDLDMLGSD | |
| | ALDDFDLDMLGSDALDDFDLDMLGSDALDDFDLDMLGS | |
| 49 | MALPVTALLLPLALLLHAARPEQKLISEEDLDIQMTQTTSSLSASLGDRVT | pSTS049 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with EFNB1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKLRKRHRKHMKLLSSIEQACDICRLKKLKCSKEKPKCAK | |
| | CLKNNWECRYSPKTKRSPLTRAHLTEVESRLERLEQLFLLIFPREDLDMIL | |
| | KMDSLQDIKALLTGLFVQDNVNKDAVTDRLASVETDMPLTLRQHRISAT | |
| | SSSEESSNKGQRQLTVSAAAGGSGGSGGSDALDDFDLDMLGSDALDDFD | |
| | LDMLGSDALDDFDLDMLGSDALDDFDLDMLGS | |
| 50 | MALPVTALLLPLALLLHAARPEQKLISEEDLDIQMTQTTSSLSASLGDRVT | pSTS050 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CD44 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRRCGQKKKMKLLSSIEQACDICRLKKLKCSKEKPKCAK | |
| | CLKNNWECRYSPKTKRSPLTRAHLTEVESRLERLEQLFLLIFPREDLDMIL | |
| | KMDSLQDIKALLTGLFVQDNVNKDAVTDRLASVETDMPLTLRQHRISAT | |
| | SSSEESSNKGQRQLTVSAAAGGSGGSGGSDALDDFDLDMLGSDALDDFD | |
| | LDMLGSDALDDFDLDMLGSDALDDFDLDMLGS | |
| 51 | MALPVTALLLPLALLLHAARPEQKLISEEDLDIQMTQTTSSLSASLGDRVT | pSTS051 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CLSTN1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRIRAAHRRTMRMKLLSSIEQACDICRLKKLKCSKEKPKC | |
| | AKCLKNNWECRYSPKTKRSPLTRAHLTEVESRLERLEQLFLLIFPREDLD | |
| | MILKMDSLQDIKALLTGLFVQDNVNKDAVTDRLASVETDMPLTLRQHRI | |
| | SATSSSEESSNKGQRQLTVSAAAGGSGGSGGSDALDDFDLDMLGSDALD | |
| | DFDLDMLGSDALDDFDLDMLGSDALDDFDLDMLGS | |
| 52 | MALPVTALLLPLALLLHAARPEQKLISEEDLDIQMTQTTSSLSASLGDRVT | pSTS052 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with LRP8 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRNWKRKNTKMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 53 | MALPVTALLLPLALLLHAARPEQKLISEEDLDIQMTQTTSSLSASLGDRVT | pSTS053 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with |
| | GVIWGSETTYYNSALKSRLTIIKDNSKSQVFLKMNSLQTDDTAIYYCAKH | PCDHGC3 |
| | YYYGGSYAMDYWGQGTSVTVSSILDYSFGGGAGRDIPPPLIEEACELPEC | STS |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKVYKWKQSRMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 54 | MALPVTALLLPLALLLHAARPEQKLISEEDLDIQMTQTTSSLSASLGDRVT | pSTS054 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with NRG1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKTKKQRKKLHDRLRMKLLSSIEQACDICRLKKLKCSKEK | |
| | PKCAKCLKNNWECRYSPKTKRSPLTRAHLTEVESRLERLEQLFLLIFPRED | |
| | LDMILKMDSLQDIKALLTGLFVQDNVNKDAVTDRLASVETDMPLTLRQH | |
| | RISATSSSEESSNKGQRQLTVSAAAGGSGGSGGSDALDDFDLDMLGSDAL | |
| | DDFDLDMLGSDALDDFDLDMLGSDALDDFDLDMLGS | |
| 55 | MALPVTALLLPLALLLHAARPEQKLISEEDLDIQMTQTTSSLSASLGDRVT | pSTS055 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with LRP1B |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRKRRTKTIRRMKLLSSIEQACDICRLKKLKCSKEKPKC | |
| | AKCLKNNWECRYSPKTKRSPLTRAHLTEVESRLERLEQLFLLIFPREDLD | |
| | MILKMDSLQDIKALLTGLFVQDNVNKDAVTDRLASVETDMPLTLRQHRI | |
| | SATSSSEESSNKGQRQLTVSAAAGGSGGSGGSDALDDFDLDMLGSDALD | |
| | DFDLDMLGSDALDDFDLDMLGSDALDDFDLDMLGS | |
| 56 | MALPVTALLLPLALLLHAARPEQKLISEEDLDIQMTQTTSSLSASLGDRVT | pSTS056 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with JAG2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKRRKERERSRLPRMKLLSSIEQACDICRLKKLKCSKEKP | |
| | KCAKCLKNNWECRYSPKTKRSPLTRAHLTEVESRLERLEQLFLLIFPRED | |
| | LDMILKMDSLQDIKALLTGLFVQDNVNKDAVTDRLASVETDMPLTLRQH | |
| | RISATSSSEESSNKGQRQLTVSAAAGGSGGSGGSDALDDFDLDMLGSDAL | |
| | DDFDLDMLGSDALDDFDLDMLGSDALDDFDLDMLGS | |
| 57 | MALPVTALLLPLALLLHAARPEQKLISEEDLDIQMTQTTSSLSASLGDRVT | pSTS057 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with EFNB2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKYRRRHRKHMKLLSSIEQACDICRLKKLKCSKEKPKCAK | |
| | CLKNNWECRYSPKTKRSPLTRAHLTEVESRLERLEQLFLLIFPREDLDMIL | |
| | KMDSLQDIKALLTGLFVQDNVNKDAVTDRLASVETDMPLTLRQHRISAT | |
| | SSSEESSNKGQRQLTVSAAAGGSGGSGGSDALDDFDLDMLGSDALDDFD | |
| | LDMLGSDALDDFDLDMLGSDALDDFDLDMLGS | |
| 58 | MALPVTALLLPLALLLHAARPEQKLISEEDLDIQMTQTTSSLSASLGDRVT | pSTS058 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with DLL1 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRLRLQKHRMKLLSSIEQACDICRLKKLKCSKEKPKCAKC | |
| | LKNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 59 | MALPVTALLLPLALLLHAARPEQKLISEEDLDIQMTQTTSSLSASLGDRVT | pSTS059 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CLSTN2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRVRIAHQHMKLLSSIEQACDICRLKKLKCSKEKPKCAKC | |
| | LKNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 60 | MALPVTALLLPLALLLHAARPEQKLISEEDLDIQMTQTTSSLSASLGDRVT | pSTS060 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with EPCAM |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKKRMAKYEKMKLLSSIEQACDICRLKKLKCSKEKPKC | |
| | AKCLKNNWECRYSPKTKRSPLTRAHLTEVESRLERLEQLFLLIFPREDLD | |
| | MILKMDSLQDIKALLTGLFVQDNVNKDAVTDRLASVETDMPLTLRQHRI | |
| | SATSSSEESSNKGQRQLTVSAAAGGSGGSGGSDALDDFDLDMLGSDALD | |
| | DFDLDMLGSDALDDFDLDMLGSDALDDFDLDMLGS | |
| 61 | MALPVTALLLPLALLLHAARPEQKLISEEDLDIQMTQTTSSLSASLGDRVT | pSTS061 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with ErbB4 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRKSIKKKRALRRMKLLSSIEQACDICRLKKLKCSKEKP | |
| | KCAKCLKNNWECRYSPKTKRSPLTRAHLTEVESRLERLEQLFLLIFPRED | |
| | LDMILKMDSLQDIKALLTGLFVQDNVNKDAVTDRLASVETDMPLTLRQH | |
| | RISATSSSEESSNKGQRQLTVSAAAGGSGGSGGSDALDDFDLDMLGSDAL | |
| | DDFDLDMLGSDALDDFDLDMLGSDALDDFDLDMLGS | |
| 62 | MALPVTALLLPLALLLHAARPEQKLISEEDLDIQMTQTTSSLSASLGDRVT | pSTS062 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with KCNE3 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRSRKVDKRMKLLSSIEQACDICRLKKLKCSKEKPKCAKC | |
| | LKNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 63 | MALPVTALLLPLALLLHAARPEQKLISEEDLDIQMTQTTSSLSASLGDRVT | pSTS063 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with CDH2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRRDKERQAKMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 64 | MALPVTALLLPLALLLHAARPEQKLISEEDLDIQMTQTTSSLSASLGDRVT | pSTS064 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with NRG2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKTKKQRKQMHNHLRMKLLSSIEQACDICRLKKLKCSKE | |
| | KPKCAKCLKNNWECRYSPKTKRSPLTRAHLTEVESRLERLEQLFLLIFPRE | |
| | DLDMILKMDSLQDIKALLTGLFVQDNVNKDAVTDRLASVETDMPLTLRQ | |
| | HRISATSSSEESSNKGQRQLTVSAAAGGSGGSGGSDALDDFDLDMLGSD | |
| | ALDDFDLDMLGSDALDDFDLDMLGSDALDDFDLDMLGS | |
| 65 | MALPVTALLLPLALLLHAARPEQKLISEEDLDIQMTQTTSSLSASLGDRVT | pSTS065 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with PTPRK |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKKSKLAKKRKMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 66 | MALPVTALLLPLALLLHAARPEQKLISEEDLDIQMTQTTSSLSASLGDRVT | pSTS066 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with BTC STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSHPLRKRRKRKKKMKLLSSIEQACDICRLKKLKCSKEKPK | |
| | CAKCLKNNWECRYSPKTKRSPLTRAHLTEVESRLERLEQLFLLIFPREDL | |
| | DMILKMDSLQDIKALLTGLFVQDNVNKDAVTDRLASVETDMPLTLRQH | |
| | RISATSSSEESSNKGQRQLTVSAAAGGSGGSGGSDALDDFDLDMLGSDAL | |
| | DDFDLDMLGSDALDDFDLDMLGSDALDDFDLDMLGS | |
| 67 | MALPVTALLLPLALLLHAARPEQKLISEEDLDIQMTQTTSSLSASLGDRVT | pSTS067 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with EPHA4 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRRSKYSKAKMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 68 | MALPVTALLLPLALLLHAARPEQKLISEEDLDIQMTQTTSSLSASLGDRVT | pSTS068 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with IL1R2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSHRRCKHRTGKMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 69 | MALPVTALLLPLALLLHAARPEQKLISEEDLDIQMTQTTSSLSASLGDRVT | pSTS069 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with KCNE4 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKSKRREKKMKLLSSIEQACDICRLKKLKCSKEKPKCAKC | |
| | LKNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 70 | MALPVTALLLPLALLLHAARPEQKLISEEDLDIQMTQTTSSLSASLGDRVT | pSTS070 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with SCN2B |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKCVRRKKEQKMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 71 | MALPVTALLLPLALLLHAARPEQKLISEEDLDIQMTQTTSSLSASLGDRVT | pSTS071 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with Nradd |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKCWRSHKQRMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 72 | MALPVTALLLPLALLLHAARPEQKLISEEDLDIQMTQTTSSLSASLGDRVT | pSTS072 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with PTPRM |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKKRKLAKKRKMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 73 | MALPVTALLLPLALLLHAARPEQKLISEEDLDIQMTQTTSSLSASLGDRVT | pIZ618 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with Notch2 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRKRKHMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 74 | MALPVTALLLPLALLLHAARPEQKLISEEDLDIQMTQTTSSLSASLGDRVT | pIZ619 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with Notch3 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRKREHMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 75 | MALPVTALLLPLALLLHAARPEQKLISEEDLDIQMTQTTSSLSASLGDRVT | pIZ620 |
| | ISCRASQDISKYLNWYQQKPDGTVKLLIYHTSRLHSGVPSRFSGSGSGTD | Human |
| | YSLTISNLEQEDIATYFCQQGNTLPYTFGGGTKLEITGGGGSGGGGSGGG | SynNotch1 |
| | GSEVKLQESGPGLVAPSQSLSVTCTVSGVSLPDYGVSWIRQPPRKGLEWL | with Notch4 |
| | GVIWGSETTYYNSALKSRLTIIKDNSKSQVFLKMNSLQTDDTAIYYCAKH | STS |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRRRRREHMKLLSSIEQACDICRLKKLKCSKEKPKCAKCL | |
| | KNNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDFDL | |
| | DMLGSDALDDFDLDMLGSDALDDFDLDMLGS | |
| 76 | MALPVTALLLPLALLLHAARPEQKLISEEDLDIQMTQTTSSLSASLGDRVT | pSTS106 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with CXCL16 |
| | GSEVKLQESGPGLVAPSQSLSVTCTVSGVSLPDYGVSWIRQPPRKGLEWL | STS |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKRRRMKLLSSIEQACDICRLKKL | |
| | KCSKEKPKCAKCLKNNWECRYSPKTKRSPLTRAHLTEVESRLERLEQLFL | |
| | LIFPREDLDMILKMDSLQDIKALLTGLFVQDNVNKDAVTDRLASVETDM | |
| | PLTLRQHRISATSSSEESSNKGQRQLTVSAAAGGSGGSGGSDALDDFDLD | |
| | MLGSDALDDFDLDMLGSDALDDFDLDMLGSDALDDFDLDMLGS | |
| 77 | MALPVTALLLPLALLLHAARPEQKLISEEDLDIQMTQTTSSLSASLGDRVT | pSTS108 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#8 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSRKKRKGKMKLLSSIEQACDICRL | |
| | KKLKCSKEKPKCAKCLKNNWECRYSPKTKRSPLTRAHLTEVESRLERLE | |
| | QLFLLIFPREDLDMILKMDSLQDIKALLTGLFVQDNVNKDAVTDRLASVE | |
| | TDMPLTLRQHRISATSSSEESSNKGQRQLTVSAAAGGSGGSGGSDALDDF | |
| | DLDMLGSDALDDFDLDMLGSDALDDFDLDMLGSDALDDFDLDMLGS | |
| 78 | MALPVTALLLPLALLLHAARPEQKLISEEDLDIQMTQTTSSLSASLGDRVT | pSTS113 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS# 13 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKQQRIKMKLLSSIEQACDICRLK | |
| | KLKCSKEKPKCAKCLKNNWECRYSPKTKRSPLTRAHLTEVESRLERLEQ | |
| | LFLLIFPREDLDMILKMDSLQDIKALLTGLFVQDNVNKDAVTDRLASVET | |
| | DMPLTLRQHRISATSSSEESSNKGQRQLTVSAAAGGSGGSGGSDALDDFD | |
| | LDMLGSDALDDFDLDMLGSDALDDFDLDMLGSDALDDFDLDMLGS | |
| 79 | MALPVTALLLPLALLLHAARPEQKLISEEDLDIQMTQTTSSLSASLGDRVT | pSTS124 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#24 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKRKRTHMKLLSSIEQACDICRLK | |
| | KLKCSKEKPKCAKCLKNNWECRYSPKTKRSPLTRAHLTEVESRLERLEQ | |
| | LFLLIFPREDLDMILKMDSLQDIKALLTGLFVQDNVNKDAVTDRLASVET | |
| | DMPLTLRQHRISATSSSEESSNKGQRQLTVSAAAGGSGGSGGSDALDDFD | |
| | LDMLGSDALDDFDLDMLGSDALDDFDLDMLGSDALDDFDLDMLGS | |
| 80 | MALPVTALLLPLALLLHAARPEQKLISEEDLDIQMTQTTSSLSASLGDRVT | pSTS147 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#47 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKKGRRSYKMKLLSSIEQACDICR | |
| | LKKLKCSKEKPKCAKCLKNNWECRYSPKTKRSPLTRAHLTEVESRLERL | |
| | EQLFLLIFPREDLDMILKMDSLQDIKALLTGLFVQDNVNKDAVTDRLASV | |
| | ETDMPLTLRQHRISATSSSEESSNKGQRQLTVSAAAGGSGGSGGSDALDD | |
| | FDLDMLGSDALDDFDLDMLGSDALDDFDLDMLGSDALDDFDLDMLGS | |
| 81 | MALPVTALLLPLALLLHAARPEQKLISEEDLDIQMTQTTSSLSASLGDRVT | pSTS154 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#54 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKTKKQRKKLHDRLRMKLLSSIEQ | |
| | ACDICRLKKLKCSKEKPKCAKCLKNNWECRYSPKTKRSPLTRAHLTEVE | |
| | SRLERLEQLFLLIFPREDLDMILKMDSLQDIKALLTGLFVQDNVNKDAVT | |
| | DRLASVETDMPLTLRQHRISATSSSEESSNKGQRQLTVSAAAGGSGGSGG | |
| | SDALDDFDLDMLGSDALDDFDLDMLGSDALDDFDLDMLGSDALDDFDL | |
| | DMLGS | |
| 82 | MALPVTALLLPLALLLHAARPEQKLISEEDLDIQMTQTTSSLSASLGDRVT | pSTS155 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#55 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKRKRRTKTIRRMKLLSSIEQACDI | |
| | CRLKKLKCSKEKPKCAKCLKNNWECRYSPKTKRSPLTRAHLTEVESRLE | |
| | RLEQLFLLIFPREDLDMILKMDSLQDIKALLTGLFVQDNVNKDAVTDRLA | |
| | SVETDMPLTLRQHRISATSSSEESSNKGQRQLTVSAAAGGSGGSGGSDAL | |
| | DDFDLDMLGSDALDDFDLDMLGSDALDDFDLDMLGSDALDDFDLDML | |
| | GS | |
| 83 | MALPVTALLLPLALLLHAARPEQKLISEEDLDIQMTQTTSSLSASLGDRVT | pSTS156 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#56 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSRKRRKERERSRLPRMKLLSSIEQ | |
| | ACDICRLKKLKCSKEKPKCAKCLKNNWECRYSPKTKRSPLTRAHLTEVE | |
| | SRLERLEQLFLLIFPREDLDMILKMDSLQDIKALLTGLFVQDNVNKDAVT | |
| | DRLASVETDMPLTLRQHRISATSSSEESSNKGQRQLTVSAAAGGSGGSGG | |
| | SDALDDFDLDMLGSDALDDFDLDMLGSDALDDFDLDMLGSDALDDFDL | |
| | DMLGS | |
| 84 | MALPVTALLLPLALLLHAARPEQKLISEEDLDIQMTQTTSSLSASLGDRVT | pSTS160 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#60 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSRKKRMAKYEKMKLLSSIEQACDI | |
| | CRLKKLKCSKEKPKCAKCLKNNWECRYSPKTKRSPLTRAHLTEVESRLE | |
| | RLEQLFLLIFPREDLDMILKMDSLQDIKALLTGLFVQDNVNKDAVTDRLA | |
| | SVETDMPLTLRQHRISATSSSEESSNKGQRQLTVSAAAGGSGGSGGSDAL | |
| | DDFDLDMLGSDALDDFDLDMLGSDALDDFDLDMLGSDALDDFDLDML | |
| | GS | |
| 85 | MALPVTALLLPLALLLHAARPEQKLISEEDLDIQMTQTTSSLSASLGDRVT | pSTS164 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#64 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKTKKQRKQMHNHLRMKLLSSIE | |
| | QACDICRLKKLKCSKEKPKCAKCLKNNWECRYSPKTKRSPLTRAHLTEV | |
| | ESRLERLEQLFLLIFPREDLDMILKMDSLQDIKALLTGLFVQDNVNKDAV | |
| | TDRLASVETDMPLTLRQHRISATSSSEESSNKGQRQLTVSAAAGGSGGSG | |
| | GSDALDDFDLDMLGSDALDDFDLDMLGSDALDDFDLDMLGSDALDDFD | |
| | LDMLGS | |
| 86 | MALPVTALLLPLALLLHAARPEQKLISEEDLDIQMTQTTSSLSASLGDRVT | pSTS165 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#65 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKKSKLAKKRKMKLLSSIEQACDI | |
| | CRLKKLKCSKEKPKCAKCLKNNWECRYSPKTKRSPLTRAHLTEVESRLE | |
| | RLEQLFLLIFPREDLDMILKMDSLQDIKALLTGLFVQDNVNKDAVTDRLA | |
| | SVETDMPLTLRQHRISATSSSEESSNKGQRQLTVSAAAGGSGGSGGSDAL | |
| | DDFDLDMLGSDALDDFDLDMLGSDALDDFDLDMLGSDALDDFDLDML | |
| | GS | |
| 87 | MALPVTALLLPLALLLHAARPEQKLISEEDLDIQMTQTTSSLSASLGDRVT | pSTS172 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | with STS#72 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKKRKLAKKRKMKLLSSIEQACDI | |
| | CRLKKLKCSKEKPKCAKCLKNNWECRYSPKTKRSPLTRAHLTEVESRLE | |
| | RLEQLFLLIFPREDLDMILKMDSLQDIKALLTGLFVQDNVNKDAVTDRLA | |
| | SVETDMPLTLRQHRISATSSSEESSNKGQRQLTVSAAAGGSGGSGGSDAL | |
| | DDFDLDMLGSDALDDFDLDMLGSDALDDFDLDMLGSDALDDFDLDML | |
| | GS | |
| 88 | MALPVTALLLPLALLLHAARPEQKLISEEDLDIQMTQTTSSLSASLGDRVT | pSTS206 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#6 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKRRRMKLLSSIEQACDICRLKKLKCSKE | |
| | KPKCAKCLKNNWECRYSPKTKRSPLTRAHLTEVESRLERLEQLFLLIFPRE | |
| | DLDMILKMDSLQDIKALLTGLFVQDNVNKDAVTDRLASVETDMPLTLRQ | |
| | HRISATSSSEESSNKGQRQLTVSAAAGGSGGSGGSDALDDFDLDMLGSD | |
| | ALDDFDLDMLGSDALDDFDLDMLGSDALDDFDLDMLGS | |
| 89 | MALPVTALLLPLALLLHAARPEQKLISEEDLDIQMTQTTSSLSASLGDRVT | pSTS208 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#8 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSRKKRKGKMKLLSSIEQACDICRLKKLKC | |
| | SKEKPKCAKCLKNNWECRYSPKTKRSPLTRAHLTEVESRLERLEQLFLLI | |
| | FPREDLDMILKMDSLQDIKALLTGLFVQDNVNKDAVTDRLASVETDMPL | |
| | TLRQHRISATSSSEESSNKGQRQLTVSAAAGGSGGSGGSDALDDFDLDML | |
| | GSDALDDFDLDMLGSDALDDFDLDMLGSDALDDFDLDMLGS | |
| 90 | MALPVTALLLPLALLLHAARPEQKLISEEDLDIQMTQTTSSLSASLGDRVT | pSTS213 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS# 13 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKQQRIKMKLLSSIEQACDICRLKKLKCSK | |
| | EKPKCAKCLKNNWECRYSPKTKRSPLTRAHLTEVESRLERLEQLFLLIFPR | |
| | EDLDMILKMDSLQDIKALLTGLFVQDNVNKDAVTDRLASVETDMPLTLR | |
| | QHRISATSSSEESSNKGQRQLTVSAAAGGSGGSGGSDALDDFDLDMLGS | |
| | DALDDFDLDMLGSDALDDFDLDMLGSDALDDFDLDMLGS | |
| 91 | MALPVTALLLPLALLLHAARPEQKLISEEDLDIQMTQTTSSLSASLGDRVT | pSTS224 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#24 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKRKRTHMKLLSSIEQACDICRLKKLKCS | |
| | KEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDML | |
| | GSDALDDFDLDMLGSDALDDFDLDMLGSDALDDFDLDMLGS | |
| 92 | MALPVTALLLPLALLLHAARPEQKLISEEDLDIQMTQTTSSLSASLGDRVT | pSTS247 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#47 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKKGRRSYKMKLLSSIEQACDICRLKKLK | |
| | CSKEKPKCAKCLKNNWECRYSPKTKRSPLTRAHLTEVESRLERLEQLFLL | |
| | IFPREDLDMILKMDSLQDIKALLTGLFVQDNVNKDAVTDRLASVETDMP | |
| | LTLRQHRISATSSSEESSNKGQRQLTVSAAAGGSGGSGGSDALDDFDLDM | |
| | LGSDALDDFDLDMLGSDALDDFDLDMLGSDALDDFDLDMLGS | |
| 93 | MALPVTALLLPLALLLHAARPEQKLISEEDLDIQMTQTTSSLSASLGDRVT | pSTS254 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#54 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKTKKQRKKLHDRLRMKLLSSIEQACDIC | |
| | RLKKLKCSKEKPKCAKCLKNNWECRYSPKTKRSPLTRAHLTEVESRLER | |
| | LEQLFLLIFPREDLDMILKMDSLQDIKALLTGLFVQDNVNKDAVTDRLAS | |
| | VETDMPLTLRQHRISATSSSEESSNKGQRQLTVSAAAGGSGGSGGSDALD | |
| | DFDLDMLGSDALDDFDLDMLGSDALDDFDLDMLGSDALDDFDLDMLGS | |
| 94 | MALPVTALLLPLALLLHAARPEQKLISEEDLDIQMTQTTSSLSASLGDRVT | pSTS255 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#55 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKRKRRTKTIRRMKLLSSIEQACDICRLKK | |
| | LKCSKEKPKCAKCLKNNWECRYSPKTKRSPLTRAHLTEVESRLERLEQLF | |
| | LLIFPREDLDMILKMDSLQDIKALLTGLFVQDNVNKDAVTDRLASVETD | |
| | MPLTLRQHRISATSSSEESSNKGQRQLTVSAAAGGSGGSGGSDALDDFDL | |
| | DMLGSDALDDFDLDMLGSDALDDFDLDMLGSDALDDFDLDMLGS | |
| 95 | MALPVTALLLPLALLLHAARPEQKLISEEDLDIQMTQTTSSLSASLGDRVT | pSTS256 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#56 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSRKRRKERERSRLPRMKLLSSIEQACDICR | |
| | LKKLKCSKEKPKCAKCLKNNWECRYSPKTKRSPLTRAHLTEVESRLERL | |
| | EQLFLLIFPREDLDMILKMDSLQDIKALLTGLFVQDNVNKDAVTDRLASV | |
| | ETDMPLTLRQHRISATSSSEESSNKGQRQLTVSAAAGGSGGSGGSDALDD | |
| | FDLDMLGSDALDDFDLDMLGSDALDDFDLDMLGSDALDDFDLDMLGS | |
| 96 | MALPVTALLLPLALLLHAARPEQKLISEEDLDIQMTQTTSSLSASLGDRVT | pSTS260 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#60 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSRKKRMAKYEKMKLLSSIEQACDICRLKK | |
| | LKCSKEKPKCAKCLKNNWECRYSPKTKRSPLTRAHLTEVESRLERLEQLF | |
| | LLIFPREDLDMILKMDSLQDIKALLTGLFVQDNVNKDAVTDRLASVETD | |
| | MPLTLRQHRISATSSSEESSNKGQRQLTVSAAAGGSGGSGGSDALDDFDL | |
| | DMLGSDALDDFDLDMLGSDALDDFDLDMLGSDALDDFDLDMLGS | |
| 97 | MALPVTALLLPLALLLHAARPEQKLISEEDLDIQMTQTTSSLSASLGDRVT | pSTS264 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#64 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKTKKQRKQMHNHLRMKLLSSIEQACDI | |
| | CRLKKLKCSKEKPKCAKCLKNNWECRYSPKTKRSPLTRAHLTEVESRLE | |
| | RLEQLFLLIFPREDLDMILKMDSLQDIKALLTGLFVQDNVNKDAVTDRLA | |
| | SVETDMPLTLRQHRISATSSSEESSNKGQRQLTVSAAAGGSGGSGGSDAL | |
| | DDFDLDMLGSDALDDFDLDMLGSDALDDFDLDMLGSDALDDFDLDML | |
| | GS | |
| 98 | MALPVTALLLPLALLLHAARPEQKLISEEDLDIQMTQTTSSLSASLGDRVT | pSTS265 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#65 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKKSKLAKKRKMKLLSSIEQACDICRLKK | |
| | LKCSKEKPKCAKCLKNNWECRYSPKTKRSPLTRAHLTEVESRLERLEQLF | |
| | LLIFPREDLDMILKMDSLQDIKALLTGLFVQDNVNKDAVTDRLASVETD | |
| | MPLTLRQHRISATSSSEESSNKGQRQLTVSAAAGGSGGSGGSDALDDFDL | |
| | DMLGSDALDDFDLDMLGSDALDDFDLDMLGSDALDDFDLDMLGS | |
| 99 | MALPVTALLLPLALLLHAARPEQKLISEEDLDIQMTQTTSSLSASLGDRVT | pSTS272 |
| | ISCRASQDISKYLNWYQQKPDGTVKLLIYHTSRLHSGVPSRFSGSGSGTD | HingeNotch |
| | YSLTISNLEQEDIATYFCQQGNTLPYTFGGGTKLEITGGGGSGGGGSGGG | with STS#72 |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKKRKLAKKRKMKLLSSIEQACDICRLKK | |
| | LKCSKEKPKCAKCLKNNWECRYSPKTKRSPLTRAHLTEVESRLERLEQLF | |
| | LLIFPREDLDMILKMDSLQDIKALLTGLFVQDNVNKDAVTDRLASVETD | |
| | MPLTLRQHRISATSSSEESSNKGQRQLTVSAAAGGSGGSGGSDALDDFDL | |
| | DMLGSDALDDFDLDMLGSDALDDFDLDMLGSDALDDFDLDMLGS | |
| 100 | RRKK | APLP1 STS |
| 101 | RKR | APLP2 STS |
| 102 | KKK | APP STS |
| 103 | H | TGBR3 STS |
| 104 | KYKQKPK | CSF1R STS |
| 105 | KRRR | CXCL16 STS |
| 106 | RK | CX3CL1 STS |
| 107 | RKKRKGK | DAGI STS |
| 108 | RR | DCC STS |
| 109 | RISR | DNER STS |
| 110 | KCGKGAK | DSG2 STS |
| 111 | RRR | CDH1 STS |
| 112 | KQQRIK | GHR STS |
| 113 | RRK | HLA-A STS |
| 114 | K | IFNAR2 STS |
| 115 | HRKR | IGF1R STS |
| 116 | KIFK | IL1R1 STS |
| 117 | R | ERN2 STS |
| 118 | RSKKLEH | KCNE1 STS |
| 119 | KSKRREH | KCNE2 STS |
| 120 | KRNRGGK | CHL1 STS |
| 121 | KRR | LRP1 STS |
| 122 | HYRR | LRP2 STS |
| 123 | KRKRTH | PTPRF STS |
| 124 | KK | SCN1B STS |
| 125 | RKVSK | SCN3B STS |
| 126 | RKKYR | NPR3 STS |
| 127 | KR | NGFR STS |
| 128 | HH | PLXDC2 STS |
| 129 | RWKKSR | PAM STS |
| 130 | RRQRR | AGER STS |
| 131 | RHRKKR | ROBO1 STS |
| 132 | KRK | SORCS3 STS |
| 133 | KFKRR | SORCS1 STS |
| 134 | KHRR | SORL1 STS |
| 135 | RMKKK | SDC1 STS |
| 136 | RMRKK | SDC2 STS |
| 137 | RRRQKRR | SPN STS |
| 138 | RHKRK | TYR STS |
| 139 | RARR | TYRP1 STS |
| 140 | RRLRK | DCT STS |
| 141 | RRGR | VASN STS |
| 142 | RKMKR | FLT1 STS |
| 143 | RRRLRKQARAHGK | CDH5 STS |
| 144 | KRSKSRKTK | PKHD1 STS |
| 145 | RRRRHTFK | NECTIN1 STS |
| 146 | KKGRRSYK | KL STS |
| 147 | RFKKTWKLRALKEGK | IL6R STS |
| 148 | KLRKRHRKH | EFNB1 STS |
| 149 | RRRCGQKKK | CD44 STS |
| 150 | RIRAAHRRTMR | CLSTN1 STS |
| 151 | RNWKRKNTK | LRP8 STS |
| 152 | KVYKWKQSR | PCDHGC3 STS |
| 153 | KTKKQRKKLHDRLR | NRG1 STS |
| 154 | KRKRRTKTIRR | LRP1B STS |
| 155 | RKRRKERERSRLPR | JAG2 STS |
| 156 | KYRRRHRKH | EFNB2 STS |
| 157 | RLRLQKHR | DLL1 STS |
| 158 | RVRIAHQH | CLSTN2 STS |
| 159 | RKKRMAKYEK | EPCAM STS |
| 160 | RRKSIKKKRALRR | ErbB4 STS |
| 161 | RSRKVDKR | KCNE3 STS |
| 162 | KRRDKERQAK | CDH2 STS |
| 163 | KTKKQRKQMHNHLR | NRG2 STS |
| 164 | KKSKLAKKRK | PTPRK STS |
| 165 | HPLRKRRKRKKK | BTC STS |
| 166 | RRRSKYSKAK | EPHA4 STS |
| 167 | HRRCKHRTGK | IL1R2 STS |
| 168 | KSKRREKK | KCNE4 STS |
| 169 | KCVRRKKEQK | SCN2B STS |
| 170 | KCWRSHKQR | Nradd STS |
| 171 | KKRKLAKKRK | PTPRM STS |
| 172 | KRKRKH | Notch2 STS |
| 173 | RRKREH | Notch3 STS |
| 174 | RRRRREH | Notch4 STS |
| 175 | MALPVTALLLPLALLLHAARPEQKLISEEDLDIQMTQTTSSLSASLGDRVT | ECD (extra- |
| | ISCRASQDISKYLNWYQQKPDGTVKLLIYHTSRLHSGVPSRFSGSGSGTD | cellular |
| | YSLTISNLEQEDIATYFCQQGNTLPYTFGGGTKLEITGGGGSGGGGSGGG | binding |
| | GSEVKLQESGPGLVAPSQSLSVTCTVSGVSLPDYGVSWIRQPPRKGLEWL | domain) |
| | GVIWGSETTYYNSALKSRLTIIKDNSKSQVFLKMNSLQTDDTAIYYCAKH | |
| | YYYGGSYAMDYWGQGTSVTVSS | |
| 176 | MALPVTALLLPLALLLHAARP | CD8A signal |
| | | sequence |
| 177 | EQKLISEEDL | Myc tag |
| 178 | ILDYSFGGGAGRDIPPPLIEEACELPECQEDAGNKVCSLQCNNHACGWDG | SynNotch1 |
| | GDCSLNFNDPWKNCTQSLQCWKYFSDGHCDSQCNSAGCLFDGFDCQRA | JMD |
| | EGQCNPLYDQYCKDHFSDGHCDQGCNSAECEWDGLDCAEHVPERLAAG | |
| | TLVVVVLMPPEQLRNSSFHFLRELSRVLHTNVVFKRDAHGQQMIFPYYG | |
| | REEELRKHPIKRAAEGWAAPDALLGQVKASLLPGGSEGGRRRRELDPMD | |
| | VRGSIVYLEIDNRQCVQASSQCFQSATDVAAFLGALASLGSLNIPYKIEAV | |
| | QSETVEPPPPAQLH | |
| 179 | ILDYSFGGGAGRDIPPPLIEETVEPPPPAQLH | Mini Notch LP |
| 180 | TTTPAPRPPTPAPTIASQPLSLRPEAC | Hinge Notch |
| | | LP |
| 181 | FMYVAAAAFVLLFFVGCGVLLS | Notch1 TMD |
| 182 | MKLLSSIEQACDICRLKKLKCSKEKPKCAKCLKNNWECRYSPKTKRSPLT | ICD |
| | RAHLTEVESRLERLEQLFLLIFPREDLDMILKMDSLQDIKALLTGLFVQDN | |
| | VNKDAVTDRLASVETDMPLTLRQHRISATSSSEESSNKGQRQLTVSAAA | |
| | GGSGGSGGSDALDDFDLDMLGSDALDDFDLDMLGSDALDDFDLDMLGS | |
| | DALDDFDLDMLGS | |
| 183 | LYTAPPSTPPATSLTPERTQ | Notch2 JMD |
| 184 | APAAAPEVSEEPRPLEPPEPSVPL | Notch3 JMD |
| 185 | VHPHAGTAPPANQLPW | Notch4 JMD |
| 186 | DYKDHDGDYKDHDIDYKDDDDKPVEPPLPSQLH | 3x Flag + 11 |
| | | Notch1 JMD |
| | | aa |
| 187 | GGSGGSGGSGGSGGSGGSLH | (GGS)3 |
| | | Minimal |
| | | Linker linker |
| 188 | GGSGGSGGSPLGVRGKGGSGGSGGSLH | (GGS)3 with |
| | | MMP9 site |
| 189 | GGSGGSGGSSPLAQAVRSSSRGGSGGSGGSLH | (GGS)3 with |
| | | ADAM 17 site |
| 190 | TTTPAPRPPTPAPTIASQPLSLRPEACRPAAGGAVHTRGLDFACD | CD8A Hinge |
| | | linker |
| 191 | TTTPAPRPPTPAPTIASQPLSLRPEAC | CD8A |
| | | truncated |
| | | Hinge linker |
| 192 | IEVMYPPPYLDNEKSNGTIIHVKGKHLCPSPLFPGPSKP | CD28 Hinge |
| | | linker |
| 193 | GGGGSGGGGSGGGGSESKYGPPCPPCP | (GGGGS)3 + |
| | | IgG4 Hinge |
| | | linker |
| 194 | LHCVGDTYPSNDRCCHECRPGNGMVSRCSRSQNTVCRPCGPGFYNDVVS | OX40 Hinge |
| | SKPCKPCTWCNLRSGSERKQLCTATQDTVCRCRAGTQPLDSYKPGVDCA | linker |
| | PCPPGHFSPGDNQACKPWTNCTLAGKHTLQPASNSSDAICEDRDPPATQP | |
| | QETQGPPARPITVQPTEAWPRTSQGPSTRPVEVPGGRA | |
| 195 | KTLEEAPSAPPQGVTVSKNDGNGTAILVSWQPPPEDTQNGMVQEYKVW | Robo Fn linker |
| | CLGNETRYHINKTVDGSTFSVVIPFLVPGIRYSVEVAASTGAGSGVKSEPQ | |
| | FIQLDAHGNPVSPEDQVSLAQQISDVVKQP | |
| 196 | KTLEEAPSAPPQGVTVSKNDGNGTAILVSWQPPPEDTQNGMVQEYKVW | Truncated Fn |
| | CLGNETRYHINKTVDGSTFSVVIPFLVPGIRYSVEVAASTGAGSGVKSEPQ | linker |
| | FIQLDVVKQP | |
| 197 | KTLEEAPSAPPQGVTVSKNDGNGTAILVSWQPPPEDTQNGMVQEYKVW | Truncated Fn |
| | CLGNETRYHINKTVDGSTFSVVIPFLVPGIRYSVEVAASTGAGSGVKSEPQ | linker + |
| | FIQLDGGSGGSGGS | (GGS)3 |
| 198 | KTLEEAPSAPPQGVTVSKNDGNGTAILVSWQPPPEDTQNGMVQEYKVW | Truncated Fn |
| | CLGNETRYHINKTVDGSTFSVVIPFLVPGIRYSVEVAASTGAGSGVKSEPQ | linker + |
| | FIQLDEPPPPAQLH | Notch 1 |
| 199 | MALPVTALLLPLALLLHAARPEQKLISEEDLDIQMTQTTSSLSASLGDRVT | MiniNotch |
| | ISCRASQDISKYLNWYQQKPDGTVKLLIYHTSRLHSGVPSRFSGSGSGTD | (2, 2, 2) |
| | YSLTISNLEQEDIATYFCQQGNTLPYTFGGGTKLEITGGGGSGGGGSGGG | |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSLYTAPPSTPPATSLTPERTQLLYLLAV | |
| | AVVIILFIILLGVIMAKRKRKHMKLLSSIEQACDICRLKKLKCSKEKPKCA | |
| | KCLKNNWECRYSPKTKRSPLTRAHLTEVESRLERLEQLFLLIFPREDLDMI | |
| | LKMDSLQDIKALLTGLFVQDNVNKDAVTDRLASVETDMPLTLRQHRISA | |
| | TSSSEESSNKGQRQLTVSAAAGGSGGSGGSDALDDFDLDMLGSDALDDF | |
| | DLDMLGSDALDDFDLDMLGSDALDDFDLDMLGS | |
| 200 | MALPVTALLLPLALLLHAARPEQKLISEEDLDIQMTQTTSSLSASLGDRVT | MiniNotch |
| | ISCRASQDISKYLNWYQQKPDGTVKLLIYHTSRLHSGVPSRFSGSGSGTD | (2, 1, 1) |
| | YSLTISNLEQEDIATYFCQQGNTLPYTFGGGTKLEITGGGGSGGGGSGGG | |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSLYTAPPSTPPATSLTPERTQFMYVAAA | |
| | AFVLLFFVGCGVLLSRKRRRMKLLSSIEQACDICRLKKLKCSKEKPKCAK | |
| | CLKNNWECRYSPKTKRSPLTRAHLTEVESRLERLEQLFLLIFPREDLDMIL | |
| | KMDSLQDIKALLTGLFVQDNVNKDAVTDRLASVETDMPLTLRQHRISAT | |
| | SSSEESSNKGQRQLTVSAAAGGSGGSGGSDALDDFDLDMLGSDALDDFD | |
| | LDMLGSDALDDFDLDMLGSDALDDFDLDMLGS | |
| 201 | MALPVTALLLPLALLLHAARPEQKLISEEDLDIQMTQTTSSLSASLGDRVT | MiniNotch |
| | ISCRASQDISKYLNWYQQKPDGTVKLLIYHTSRLHSGVPSRFSGSGSGTD | (2, 1, 2) |
| | YSLTISNLEQEDIATYFCQQGNTLPYTFGGGTKLEITGGGGSGGGGSGGG | |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSFMYVAAAAFVLLFFVGCGVLLSKRK | |
| | RKHMKLLSSIEQACDICRLKKLKCSKEKPKCAKCLKNNWECRYSPKTKR | |
| | SPLTRAHLTEVESRLERLEQLFLLIFPREDLDMILKMDSLQDIKALLTGLF | |
| | VQDNVNKDAVTDRLASVETDMPLTLRQHRISATSSSEESSNKGQRQLTV | |
| | SAAAGGSGGSGGSDALDDFDLDMLGSDALDDFDLDMLGSDALDDFDLD | |
| | MLGSDALDDFDLDMLGS | |
| 202 | MALPVTALLLPLALLLHAARPEQKLISEEDLDIQMTQTTSSLSASLGDRVT | SynNotch |
| | ISCRASQDISKYLNWYQQKPDGTVKLLIYHTSRLHSGVPSRFSGSGSGTD | (1, 1, 1) |
| | YSLTISNLEQEDIATYFCQQGNTLPYTFGGGTKLEITGGGGSGGGGSGGG | |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSRKRRRMKLLSSIEQACDICRLKKLKCSKEKPKCAKCLKN | |
| | NWECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSEE | |
| | SSNKGQRQLTVSAAAGGSGGSGGSDALDDFDLDMLGSDALDDFDLDML | |
| | GSDALDDFDLDMLGSDALDDFDLDMLGS | |
| 203 | MALPVTALLLPLALLLHAARPEQKLISEEDLDIQMTQTTSSLSASLGDRVT | pIZ618 |
| | ISCRASQDISKYLNWYQQKPDGTVKLLIYHTSRLHSGVPSRFSGSGSGTD | SynNotch |
| | YSLTISNLEQEDIATYFCQQGNTLPYTFGGGTKLEITGGGGSGGGGSGGG | (1, 1, 2) |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEEACELPEC | |
| | QEDAGNKVCSLQCNNHACGWDGGDCSLNFNDPWKNCTQSLQCWKYFS | |
| | DGHCDSQCNSAGCLFDGFDCQRAEGQCNPLYDQYCKDHFSDGHCDQGC | |
| | NSAECEWDGLDCAEHVPERLAAGTLVVVVLMPPEQLRNSSFHFLRELSR | |
| | VLHTNVVFKRDAHGQQMIFPYYGREEELRKHPIKRAAEGWAAPDALLG | |
| | QVKASLLPGGSEGGRRRRELDPMDVRGSIVYLEIDNRQCVQASSQCFQSA | |
| | TDVAAFLGALASLGSLNIPYKIEAVQSETVEPPPPAQLHFMYVAAAAFVL | |
| | LFFVGCGVLLSKRKRKHMKLLSSIEQACDICRLKKLKCSKEKPKCAKCLK | |
| | NNWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLD | |
| | MLGSDALDDFDLDMLGSDALDDFDLDMLGS | |
| 204 | MALPVTALLLPLALLLHAARPEQKLISEEDLDIQMTQTTSSLSASLGDRVT | pIZ343 Hinge |
| | ISCRASQDISKYLNWYQQKPDGTVKLLIYHTSRLHSGVPSRFSGSGSGTD | Notch (1, 1, 1) |
| | YSLTISNLEQEDIATYFCQQGNTLPYTFGGGTKLEITGGGGSGGGGSGGG | |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSRKRRRMKLLSSIEQACDICRLKKLKCSK | |
| | EKPKCAKCLKNNWECRYSPKTKRSPLTRAHLTEVESRLERLEQLFLLIFPR | |
| | EDLDMILKMDSLQDIKALLTGLFVQDNVNKDAVTDRLASVETDMPLTLR | |
| | QHRISATSSSEESSNKGQRQLTVSAAAGGSGGSGGSDALDDFDLDMLGS | |
| | DALDDFDLDMLGSDALDDFDLDMLGSDALDDFDLDMLGS | |
| 205 | MALPVTALLLPLALLLHAARPEQKLISEEDLDIQMTQTTSSLSASLGDRVT | pIZ361 Hinge |
| | ISCRASQDISKYLNWYQQKPDGTVKLLIYHTSRLHSGVPSRFSGSGSGTD | Notch (1, 1, 2) |
| | YSLTISNLEQEDIATYFCQQGNTLPYTFGGGTKLEITGGGGSGGGGSGGG | |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEACF | |
| | MYVAAAAFVLLFFVGCGVLLSKRKRKHMKLLSSIEQACDICRLKKLKCS | |
| | KEKPKCAKCLKNNWECRYSPKTKRSPLTRAHLTEVESRLERLEQLFLLIF | |
| | PREDLDMILKMDSLQDIKALLTGLFVQDNVNKDAVTDRLASVETDMPLT | |
| | LRQHRISATSSSEESSNKGQRQLTVSAAAGGSGGSGGSDALDDFDLDML | |
| | GSDALDDFDLDMLGSDALDDFDLDMLGSDALDDFDLDMLGS | |
| 206 | LLYLLAVAVVIILFIILLGVIMA | Notch2 TMD |
| 207 | RKRRR | Notch 1 STS |
| 208 | MALPVTALLLPLALLLHAARPEQKLISEEDLDIQMTQTTSSLSASLGDRVT | pRay050 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | (1, 1, 1) |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSRKRRRMKLLSSIEQACDICRLKK | |
| | LKCSKEKPKCAKCLKNNWECRYSPKTKRSPLTRAHLTEVESRLERLEQLF | |
| | LLIFPREDLDMILKMDSLQDIKALLTGLFVQDNVNKDAVTDRLASVETD | |
| | MPLTLRQHRISATSSSEESSNKGQRQLTVSAAAGGSGGSGGSDALDDFDL | |
| | DMLGSDALDDFDLDMLGSDALDDFDLDMLGSDALDDFDLDMLGS | |
| 209 | MALPVTALLLPLALLLHAARPEQKLISEEDLDIQMTQTTSSLSASLGDRVT | pIZ621 |
| | ISCRASQDISKYLNWYQQKPDGTVKLLIYHTSRLHSGVPSRFSGSGSGTD | miniNotch |
| | YSLTISNLEQEDIATYFCQQGNTLPYTFGGGTKLEITGGGGSGGGGSGGG | (1, 1, 2) |
| | GSEVKLQESGPGLVAPSQSLSVTCTVSGVSLPDYGVSWIRQPPRKGLEWL | |
| | GVIWGSETTYYNSALKSRLTIIKDNSKSQVFLKMNSLQTDDTAIYYCAKH | |
| | YYYGGSYAMDYWGQGTSVTVSSILDYSFGGGAGRDIPPPLIEETVEPPPP | |
| | AQLHFMYVAAAAFVLLFFVGCGVLLSKRKRKHMKLLSSIEQACDICRLK | |
| | KLKCSKEKPKCAKCLKNNWECRYSPKTKRSPLTRAHLTEVESRLERLEQ | |
| | LFLLIFPREDLDMILKMDSLQDIKALLTGLFVQDNVNKDAVTDRLASVET | |
| | DMPLTLRQHRISATSSSEESSNKGQRQLTVSAAAGGSGGSGGSDALDDFD | |
| | LDMLGSDALDDFDLDMLGSDALDDFDLDMLGSDALDDFDLDMLGS | |
| 210 | cgataccgtcgaccaaggcagctgtagatcttagccactttttaaaagaaaaggggggactggaagggctaattcac | pHR-SIN-pGK |
| | tcccaacgaagacaagatctgctttttgcttgtactgggtctctctggttagaccagatctgagcctgggagctctctgg | (the BamH1 |
| | ctaactagggaacccactgcttaagcctcaataaagcttgccttgagtgcttcaagtagtgtgtgcccgtctgttgtgtg | site is under- |
| | actctggtaactagagatccctcagacccttttagtcagtgtggaaaatctctagcagcatctagaattaattccgtgtat | lined) |
| | tctatagtgtcacctaaatcgtatgtgtatgatacataaggttatgtattaattgtagccgcgttctaacgacaatatgtac | |
| | aagcctaattgtgtagcatctggcttactgaagcagaccctatcatctctctcgtaaactgccgtcagagtcggtttggtt | |
| | ggacgaaccttctgagtttctggtaacgccgtcccgcacccggaaatggtcagcgaaccaatcagcagggtcatcg | |
| | ctagccagatcctctacgccggacgcatcgtggccggcatcaccggcgccacaggtgcggttgctggcgcctatat | |
| | cgccgacatcaccgatggggaagatcgggctcgccacttcgggctcatgagcgcttgtttcggcgtgggtatggtg | |
| | gcaggccccgtggccgggggactgttgggcgccatctccttgcatgcaccattccttgcggcggcggtgctcaacg | |
| | gcctcaacctactactgggctgcttcctaatgcaggagtcgcataagggagagcgtcgaatggtgcactctcagtac | |
| | aatctagctctgatgccgcatagttaagccagccccgacacccgccaacacccgctgacgcgccctgacgggcttg | |
| | tctgctcccggcatccgcttacagacaagctgtgaccgtctccgggagctgcatgtgtcagaggttttcaccgtcatc | |
| | accgaaacgcgcgagacgaaagggcctcgtgatacgcctatttttataggttaatgtcatgataataatggtttcttaga | |
| | cgtcaggtggcacttttcggggaaatgtgcgcggaacccctatttgtttatttttctaaatacattcaaatatgtatccgct | |
| | catgagacaataaccctgataaatgcttcaataatattgaaaaaggaagagtatgagtattcaacatttccgtgtcgccc | |
| | ttattcccttttttgcggcattttgccttcctgtttttgctcacccagaaacgctggtgaaagtaaaagatgctgaagatca | |
| | gttgggtgcacgagtgggttacatcgaactggatctcaacagcggtaagatccttgagagttttcgccccgaagaac | |
| | gttttccaatgatgagcacttttaaagttctgctatgtggcgcggtattatcccgtattgacgccgggcaagagcaactc | |
| | ggtcgccgcatacactattctcagaatgacttggttgagtactcaccagtcacagaaaagcatcttacggatggcatg | |
| | acagtaagagaattatgcagtgctgccataaccatgagtgataacactgcggccaacttacttctgacaacgatcgga | |
| | ggaccgaaggagctaaccgcttttttgcacaacatgggggatcatgtaactcgccttgatcgttgggaaccggagct | |
| | gaatgaagccataccaaacgacgagcgtgacaccacgatgcctgtagcaatggcaacaacgttgcgcaaactatta | |
| | actggcgaactacttactctagcttcccggcaacaattaatagactggatggaggcggataaagttgcaggaccactt | |
| | ctgcgctcggcccttccggctggctggtttattgctgataaatctggagccggtgagcgtgggtctcgcggtatcattg | |
| | cagcactggggccagatggtaagccctcccgtatcgtagttatctacacgacggggagtcaggcaactatggatga | |
| | acgaaatagacagatcgctgagataggtgcctcactgattaagcattggtaactgtcagaccaagtttactcatatata | |
| | ctttagattgatttaaaacttcatttttaatttaaaaggatctaggtgaagatcctttttgataatctcatgaccaaaatccctt | |
| | aacgtgagttttcgttccactgagcgtcagaccccgtagaaaagatcaaaggatcttcttgagatcctttttttctgcgcg | |
| | taatctgctgcttgcaaacaaaaaaaccaccgctaccagcggtggtttgtttgccggatcaagagctaccaactcttttt | |
| | ccgaaggtaactggcttcagcagagcgcagataccaaatactgtccttctagtgtagccgtagttaggccaccacttc | |
| | aagaactctgtagcaccgcctacatacctcgctctgctaatcctgttaccagtggctgctgccagtggcgataagtcgt | |
| | gtcttaccgggttggactcaagacgatagttaccggataaggcgcagcggtcgggctgaacggggggttcgtgca | |
| | cacagcccagcttggagcgaacgacctacaccgaactgagatacctacagcgtgagctatgagaaagcgccacg | |
| | cttcccgaagggagaaaggcggacaggtatccggtaagcggcagggtcggaacaggagagcgcacgagggag | |
| | cttccagggggaaacgcctggtatctttatagtcctgtcgggtttcgccacctctgacttgagcgtcgatttttgtgatgc | |
| | tcgtcaggggggcggagcctatggaaaaacgccagcaacgcggcctttttacggttcctggccttttgctggcctttt | |
| | gctcacatgttctttcctgcgttatcccctgattctgtggataaccgtattaccgcctttgagtgagctgataccgctcgc | |
| | cgcagccgaacgaccgagcgcagcgagtcagtgagcgaggaagcggaagagcgcccaatacgcaaaccgcct | |
| | ctccccgcgcgttggccgattcattaatgcagctgtggaatgtgtgtcagttagggtgtggaaagtccccaggctccc | |
| | cagcaggcagaagtatgcaaagcatgcatctcaattagtcagcaaccaggtgtggaaagtccccaggctccccagc | |
| | aggcagaagtatgcaaagcatgcatctcaattagtcagcaaccatagtcccgcccctaactccgcccatcccgcccc | |
| | taactccgcccagttccgcccattctccgccccatggctgactaattttttttatttatgcagaggccgaggccgcctcg | |
| | gcctctgagctattccagaagtagtgaggaggcttttttggaggcctaggcttttgcaaaaagcttggacacaagaca | |
| | ggcttgcgagatatgtttgagaataccactttatcccgcgtcagggagaggcagtgcgtaaaaagacgcggactcat | |
| | gtgaaatactggtttttagtgcgccagatctctataatctcgcgcaacctattttcccctcgaacactttttaagccgtaga | |
| | taaacaggctgggacacttcacatgagcgaaaaatacatcgtcacctgggacatgttgcagatccatgcacgtaaac | |
| | tcgcaagccgactgatgccttctgaacaatggaaaggcattattgccgtaagccgtggcggtctgtaccgggtgcgtt | |
| | actggcgcgtgaactgggtattcgtcatgtcgataccgtttgtatttccagctacgatcacgacaaccagcgcgagctt | |
| | aaagtgctgaaacgcgcagaaggcgatggcgaaggcttcatcgttattgatgacctggtggataccggtggtactgc | |
| | ggttgcgattcgtgaaatgtatccaaaagcgcactttgtcaccatcttcgcaaaaccggctggtcgtccgctggttgat | |
| | gactatgttgttgatatcccgcaagatacctggattgaacagccgtgggatatgggcgtcgtattcgtcccgccaatct | |
| | ccggtcgctaatcttttcaacgcctggcactgccgggcgttgttctttttaacttcaggcgggttacaatagtttccagta | |
| | agtattctggaggctgcatccatgacacaggcaaacctgagcgaaaccctgttcaaaccccgctttaaacatcctgaa | |
| | acctcgacgctagtccgccgctttaatcacggcgcacaaccgcctgtgcagtcggcccttgatggtaaaaccatccc | |
| | tcactggtatcgcatgattaaccgtctgatgtggatctggcgcggcattgacccacgcgaaatcctcgacgtccaggc | |
| | acgtattgtgatgagcgatgccgaacgtaccgacgatgatttatacgatacggtgattggctaccgtggcggcaactg | |
| | gatttatgagtgggccccggatctttgtgaaggaaccttacttctgtggtgtgacataattggacaaactacctacaga | |
| | gatttaaagctctaaggtaaatataaaatttttaagtgtataatgtgttaaactactgattctaattgtttgtgtattttaga | |
| | ttccaacctatggaactgatgaatgggagcagtggtggaatgcctttaatgaggaaaacctgttttgctcagaagaaatgc | |
| | catctagtgatgatgaggctactgctgactctcaacattctactcctccaaaaaagaagagaaaggtagaagacccca | |
| | aggactttccttcagaattgctaagttttttgagtcatgctgtgtttagtaatagaactcttgcttgctttgctatttacaccac | |
| | aaaggaaaaagctgcactgctatacaagaaaattatggaaaaatattctgtaacctttataagtaggcataacagttata | |
| | atcataacatactgttttttcttactccacacaggcatagagtgtctgctattaataactatgctcaaaaattgtgtaccttta | |
| | gctttttaatttgtaaaggggttaataaggaatatttgatgtatagtgccttgactagagatcataatcagccataccacat | |
| | ttgtagaggttttacttgctttaaaaaacctcccacacctccccctgaacctgaaacataaaatgaatgcaattgttgttgt | |
| | taacttgtttattgcagcttataatggttacaaataaagcaatagcatcacaaatttcacaaataaagcatttttttcactgc | |
| | attctagttgtggtttgtccaaactcatcaatgtatcttatcatgtctggatcaactggataactcaagctaaccaaaatca | |
| | tcccaaacttcccaccccataccctattaccactgccaattacctagtggtttcatttactctaaacctgtgattcctctga | |
| | attattttcattttaaagaaattgtatttgttaaatatgtactacaaacttagtagttggaagggctaattcactcccaaagaa | |
| | gacaagatatccttgatctgtggatctaccacacacaaggctacttccctgattagcagaactacacaccagggccag | |
| | gggtcagatatccactgacctttggatggtgctacaagctagtaccagttgagccagataaggtagaagaggccaat | |
| | aaaggagagaacaccagcttgttacaccctgtgagcctgcatgggatggatgacccggagagagaagtgttagagt | |
| | ggaggtttgacagccgcctagcatttcatcacgtggcccgagagctgcatccggagtacttcaagaactgctgatatc | |
| | gagcttgctacaagggactttccgctggggactttccagggaggcgtggcctgggcgggactggggagtggcga | |
| | gccctcagatcctgcatataagcagctgctttttgcctgtactgggtctctctggttagaccagatctgagcctgggag | |
| | ctctctggctaactagggaacccactgcttaagcctcaataaagcttgccttgagtgcttcaagtagtgtgtgcccgtct | |
| | gttgtgtgactctggtaactagagatccctcagacccttttagtcagtgtggaaaatctctagcagtggcgcccgaaca | |
| | gggacttgaaagcgaaagggaaaccagaggagctctctcgacgcaggactcggcttgctgaagcgcgcacggca | |
| | agaggcgaggggcggcgactggtgagtacgccaaaaattttgactagcggaggctagaaggagagagatgggtg | |
| | cgagagcgtcagtattaagcgggggagaattagatcgcgatgggaaaaaattcggttaaggccagggggaaaga | |
| | aaaaatataaattaaaacatatagtatgggcaagcagggagctagaacgattcgcagttaatcctggcctgttagaaa | |
| | catcagaaggctgtagacaaatactgggacagctacaaccatcccttcagacaggatcagaagaacttagatcattat | |
| | ataatacagtagcaaccctctattgtgtgcatcaaaggatagagataaaagacaccaaggaagctttagacaagata | |
| | gaggaagagcaaaacaaaagtaagaccaccgcacagcaagcggccggtgatcttcagacctggacgatatatatg | |
| | agggacaattggagaagtgaattatataaatataaagtagtaaaaattgaaccattaggagtagcacccaccaaggc | |
| | aaagagaagagtggtgcagagagaaaaaagagcagtgggaataggagctttgttccttgggttcttgggagcagca | |
| | ggaagcactatgggcgcagcgtcaatgacgctgacggtacaggccagacaattattgtctggtatagtgcagcagc | |
| | agaacaatttgctgagggctattgaggcgcaacagcatctgttgcaactcacagtctggggcatcaagcagctccag | |
| | gcaagaatcctggctgtggaaagatacctaaaggatcaacagctcctggggatttggggttgctctggaaaactcatt | |
| | tgcaccactgctgtgccttggaatgctagttggagtaataaatctctggaacagatttggaatcacacgacctggatgg | |
| | agtgggacagagaaattaacaattacacaagcttaatacactccttaattgaagaatcgcaaaaccagcaagaaaag | |
| | aatgaacaagaattattggaattagataaatgggcaagtttgtggaattggtttaacataacaaattggctgtggtatata | |
| | aaattattcataatgatagtaggaggcttggtaggtttaagaatagtttttgctgtactttctatagtgaatagagttaggca | |
| | gggatattcaccattatcgtttcagacccacctcccaaccccgaggggacccgacaggcccgaaggaatagaaga | |
| | agaaggtggagagagagacagagacagatccattcgattagtgaacggatctcgacggtcgccaaatggcagtatt | |
| | catccacaattttaaaagaaaaggggggattggggggtacagtgcaggggaaagaatagtagacataatagcaac | |
| | agacatacaaactaaagaattacaaaaacaaattacaaaaattcaaaattttcgggtttattacagggacagcagagat | |
| | ccagtttggatcgataagcttgatatcgaattgggtaggggaggcgcttttcccaaggcagtctggagcatgcgcttta | |
| | gcagccccgctgggcacttggcgctacacaagtggcctctggcctcgcacacattccacatccaccggtaggcgc | |
| | caaccggctccgttctttggtggccccttcgcgccaccttctactcctcccctagtcaggaagttcccccccgccccg | |
| | cagctcgcgtcgtgcaggacgtgacaaatggaagtagcacgtctcactagtctcgtgcagatggacagcaccgctg | |
| | agcaatggaagcgggtaggcctttggggcagcggccaatagcagctttgctccttcgctttctgggctcagaggctg | |
| | ggaaggggtgggtccgggggcgggctcaggggcgggctcaggggcggggcgggcgcccgaaggtcctccgg | |
| | aggcccggcattctgcacgcttcaaaagcgcacgtctgccgcgctgttctcctcttcctcatctccgggcctttcgaatt | |
| | ctcacgcgtcaagtggagcaaggcaggtggacagtggatccttgacttgcggccgcaactcccacctgcaacatgc | |
| | gtgactgactgaggccgcgactctagagtcgacctgcaggcatgcaagcttgatatcaagcttatcgataatcaacct | |
| | ctggattacaaaatttgtgaaagattgactggtattcttaactatgttgctccttttacgctatgtggatacgctgctttaatg | |
| | cctttgtatcatgctattgcttcccgtatggctttcattttctcctccttgtataaatcctggttgctgtctctttatgaggag | |
| | ttgtggcccgttgtcaggcaacgtggcgtggtgtgcactgtgtttgctgacgcaacccccactggttggggcattgcca | |
| | ccacctgtcagctcctttccgggactttcgctttccccctccctattgccacggcggaactcatcgccgcctgccttgc | |
| | ccgctgctggacaggggctcggctgttgggcactgacaattccgtggtgttgtcggggaaatcatcgtcctttccttg | |
| | gctgctcgcctgtgttgccacctggattctgcgcgggacgtccttctgctacgtcccttcggccctcaatccagcgga | |
| | ccttccttcccgcggcctgctgccggctctgcggcctcttccgcgtcttcgccttcgccctcagacgagtcggatctcc | |
| | ctttgggccgcctccccgcat | |
| 211 | PXXHy (wherein, X represents an arbitrary residue; Hy, a hydrophobic residue such | Exemplary |
| | as Leu, Ile, Val, Phe, Trp, Tyr, Val, Met, and Pro) | cleavage |
| | | sequence of |
| | | MMP-9 |
| 212 | PXXHy(S/T) (wherein, X represents an arbitrary residue; Hy, a hydrophobic residue | Exemplary |
| | such as Leu, Ile, Val, Phe, Trp, Tyr, Val, Met, and Pro) | cleavage |
| | | sequence of |
| | | MMP-9 |
| 213 | P(L/Q)GMTS | Exemplary |
| | | cleavage |
| | | sequence of |
| | | MMP-9 |
| 214 | P(L/G)GMT | Exemplary |
| | | cleavage |
| | | sequence of |
| | | MMP-9 |
| 215 | VGR | exemplary |
| | | urokinase-type |
| | | plasminogen |
| | | activator (uPA) |
| | | or tissue |
| | | plasminogen |
| | | activator (tPA) |
| | | cleavage site |
| 216 | ENLYTQS | exemplary |
| | | tobacco etch |
| | | virus (TEV) |
| | | protease |
| | | cleavage site |
| 217 | DDDDK | exemplary |
| | | enterokinase |
| | | cleavage site |
| 218 | LVPR | exemplary |
| | | thrombin |
| | | cleavage site |
| 219 | MALPVTALLLPLALLLHAARPEQKLISEEDLDIQMTQTTSSLSASLGDRVTISC | pIZ341 |
| | RASQDISKYLNWYQQKPDGTVKLLIYHTSRLHSGVPSRFSGSGSGTDYSLTIS | 556 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSTTTPAPRPPTPAPTIASQPLSLRPEACRPAAGGAVHTRGLDF | |
| | ACDFMYVAAAAFVLLFFVGCGVLLSRKRRRMKLLSSIEQACDICRLKKLKCS | |
| | KEKPKCAKCLKNNWECRYSPKTKRSPLTRAHLTEVESRLERLEQLFLLIFPRE | |
| | DLDMILKMDSLQDIKALLTGLFVQDNVNKDAVTDRLASVETDMPLTLRQHR | |
| | ISATSSSEESSNKGQRQLTVSAAAGGSGGSGGSDALDDFDLDMLGSDALDDF | |
| | DLDMLGSDALDDFDLDMLGSDALDDFDLDMLGS | |
| 220 | MALPVTALLLPLALLLHAARPEQKLISEEDLDIQMTQTTSSLSASLGDRVTISC | pIZ358 |
| | RASQDISKYLNWYQQKPDGTVKLLIYHTSRLHSGVPSRFSGSGSGTDYSLTIS | 550 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSIEVMYPPPYLDNEKSNGTIIHVKGKHLCPSPLFPGPSKPFMY | |
| | VAAAAFVLLFFVGCGVLLSRKRRRMKLLSSIEQACDICRLKKLKCSKEKPKC | |
| | AKCLKNNWECRYSPKTKRSPLTRAHLTEVESRLERLEQLFLLIFPREDLDMIL | |
| | KMDSLQDIKALLTGLFVQDNVNKDAVTDRLASVETDMPLTLRQHRISATSSS | |
| | EESSNKGQRQLTVSAAAGGSGGSGGSDALDDFDLDMLGSDALDDFDLDML | |
| | GSDALDDFDLDMLGSDALDDFDLDMLGS | |
| 221 | MALPVTALLLPLALLLHAARPEQKLISEEDLDIQMTQTTSSLSASLGDRVTISC | pIZ359 |
| | RASQDISKYLNWYQQKPDGTVKLLIYHTSRLHSGVPSRFSGSGSGTDYSLTIS | 538 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSGGGGSGGGGSGGGGSESKYGPPCPPCPFMYVAAAAFVLLF | |
| | FVGCGVLLSRKRRRMKLLSSIEQACDICRLKKLKCSKEKPKCAKCLKNNWEC | |
| | RYSPKTKRSPLTRAHLTEVESRLERLEQLFLLIFPREDLDMILKMDSLQDIKAL | |
| | LTGLFVQDNVNKDAVTDRLASVETDMPLTLRQHRISATSSSEESSNKGQRQL | |
| | TVSAAAGGSGGSGGSDALDDFDLDMLGSDALDDFDLDMLGSDALDDFDLD | |
| | MLGSDALDDFDLDMLGS | |
| 222 | MALPVTALLLPLALLLHAARPEQKLISEEDLDIQMTQTTSSLSASLGDRVTISC | pIZ360 |
| | RASQDISKYLNWYQQKPDGTVKLLIYHTSRLHSGVPSRFSGSGSGTDYSLTIS | 697 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSLHCVGDTYPSNDRCCHECRPGNGMVSRCSRSQNTVCRPCG | |
| | PGFYNDVVSSKPCKPCTWCNLRSGSERKQLCTATQDTVCRCRAGTQPLDSY | |
| | KPGVDCAPCPPGHFSPGDNQACKPWTNCTLAGKHTLQPASNSSDAICEDRDP | |
| | PATQPQETQGPPARPITVQPTEAWPRTSQGPSTRPVEVPGGRAFMYVAAAAF | |
| | VLLFFVGCGVLLSRKRRRMKLLSSIEQACDICRLKKLKCSKEKPKCAKCLKN | |
| | NWECRYSPKTKRSPLTRAHLTEVESRLERLEQLFLLIFPREDLDMILKMDSLQ | |
| | DIKALLTGLFVQDNVNKDAVTDRLASVETDMPLTLRQHRISATSSSEESSNKG | |
| | QRQLTVSAAAGGSGGSGGSDALDDFDLDMLGSDALDDFDLDMLGSDALDD | |
| | FDLDMLGSDALDDFDLDMLGS | |
| 223 | MALPVTALLLPLALLLHAARPEQKLISEEDLQVQLQQSGGGLVKPGGSLRLS | pIZ343FYIA |
| | CAASGFTFSSYAMHWVRQAPGKGLEWVAVISYDGSNKYYADSVKGRFTISR | 542 |
| | DNSKNTLYLQMDSLRAEDTAVYYCAKEGDSSRWSYDLWGRGTLVTVSSGG | |
| | GGSGGGGSGGGGSQSALTQPASVSGSPGQSITISCTGTSSDVGGYNYVSWYQ | |
| | QHPGKAPKVMIYDVTNRPSGVSNRFSGSKSGNTASLTISGLQAEDEADYYCS | |
| | SYTIASTLVVFGGGTKLTVLTTTPAPRPPTPAPTIASQPLSLRPEACFMYVAAA | |
| | AFVLLFFVGCGVLLSRKRRRMKLLSSIEQACDICRLKKLKCSKEKPKCAKCL | |
| | KNNWECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSEESSN | |
| | KGQRQLTVSAAAGGSGGSGGSDALDDFDLDMLGSDALDDFDLDMLGSDAL | |
| | DDFDLDMLGSDALDDFDLDMLGS | |
| 224 | METDTLLLWVLLLWVPGSTGDMVSKGEELFTGVVPILVELDGDVNGHKFSV | pIZ343eGFP |
| | SGEGEGDATYGKLTLKFICTTGKLPVPWPTLVTTLTYGVQCFSRYPDHMKQH | 525 |
| | DFFKSAMPEGYVQERTIFFKDDGNYKTRAEVKFEGDTLVNRIELKGIDFKED | |
| | GNILGHKLEYNYNSHNVYIMADKQKNGIKVNFKIRHNIEDGSVQLADHYQQ | |
| | NTPIGDGPVLLPDNHYLSTQSALSKDPNEKRDHMVLLEFVTAAGITLGMDEL | |
| | YKTTTPAPRPPTPAPTIASQPLSLRPEACFMYVAAAAFVLLFFVGCGVLLSRK | |
| | RRRMKLLSSIEQACDICRLKKLKCSKEKPKCAKCLKNNWECRYSPKTKRSPL | |
| | TRAHLTEVESRLERLEQLFLLIFPREDLDMILKMDSLQDIKALLTGLFVQDNV | |
| | NKDAVTDRLASVETDMPLTLRQHRISATSSSEESSNKGQRQLTVSAAAGGSG | |
| | GSGGSDALDDFDLDMLGSDALDDFDLDMLGSDALDDFDLDMLGSDALDDF | |
| | DLDMLGS | |
| 225 | MALPVTALLLPLALLLHAARPEQKLISEEDLDIQMTQTTSSLSASLGDRVTISC | pIZ342 |
| | RASQDISKYLNWYQQKPDGTVKLLIYHTSRLHSGVPSRFSGSGSGTDYSLTIS | 537 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSTTTPAPRPPTPAPTIASQPLSLRPEAFMYVAAAAFVLLFFVG | |
| | CGVLLSRKRRRMKLLSSIEQACDICRLKKLKCSKEKPKCAKCLKNNWECRYS | |
| | PKTKRSPLTRAHLTEVESRLERLEQLFLLIFPREDLDMILKMDSLQDIKALLTG | |
| | LFVQDNVNKDAVTDRLASVETDMPLTLRQHRISATSSSEESSNKGQRQLTVS | |
| | AAAGGSGGSGGSDALDDFDLDMLGSDALDDFDLDMLGSDALDDFDLDMLG | |
| | SDALDDFDLDMLGS | |
| 226 | MALPVTALLLPLALLLHAARPEQKLISEEDLDIQMTQTTSSLSASLGDRVTISC | pIZ362 |
| | RASQDISKYLNWYQQKPDGTVKLLIYHTSRLHSGVPSRFSGSGSGTDYSLTIS | 529 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSRPAAGGAVHTRGLDFACDFMYVAAAAFVLLFFVGCGVLLS | |
| | RKRRRMKLLSSIEQACDICRLKKLKCSKEKPKCAKCLKNNWECRYSPKTKRS | |
| | PLTRAHLTEVESRLERLEQLFLLIFPREDLDMILKMDSLQDIKALLTGLFVQDN | |
| | VNKDAVTDRLASVETDMPLTLRQHRISATSSSEESSNKGQRQLTVSAAAGGS | |
| | GGSGGSDALDDFDLDMLGSDALDDFDLDMLGSDALDDFDLDMLGSDALDD | |
| | FDLDMLGS | |
| 227 | MALPVTALLLPLALLLHAARPEQKLISEEDLDIQMTQTTSSLSASLGDRVTISC | pIZ363 |
| | RASQDISKYLNWYQQKPDGTVKLLIYHTSRLHSGVPSRFSGSGSGTDYSLTIS | 530 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSCRPAAGGAVHTRGLDFACDFMYVAAAAFVLLFFVGCGVL | |
| | LSRKRRRMKLLSSIEQACDICRLKKLKCSKEKPKCAKCLKNNWECRYSPKTK | |
| | RSPLTRAHLTEVESRLERLEQLFLLIFPREDLDMILKMDSLQDIKALLTGLFVQ | |
| | DNVNKDAVTDRLASVETDMPLTLRQHRISATSSSEESSNKGQRQLTVSAAAG | |
| | GSGGSGGSDALDDFDLDMLGSDALDDFDLDMLGSDALDDFDLDMLGSDAL | |
| | DDFDLDMLGS | |
| 228 | MALPVTALLLPLALLLHAARPEQKLISEEDLQVQLQQSGGGLVKPGGSLRLS | pIZ361FYIA |
| | CAASGFTFSSYAMHWVRQAPGKGLEWVAVISYDGSNKYYADSVKGRFTISR | 543 |
| | DNSKNTLYLQMDSLRAEDTAVYYCAKEGDSSRWSYDLWGRGTLVTVSSGG | |
| | GGSGGGGSGGGGSQSALTQPASVSGSPGQSITISCTGTSSDVGGYNYVSWYQ | |
| | QHPGKAPKVMIYDVTNRPSGVSNRFSGSKSGNTASLTISGLQAEDEADYYCS | |
| | SYTIASTLVVFGGGTKLTVLTTTPAPRPPTPAPTIASQPLSLRPEACFMYVAAA | |
| | AFVLLFFVGCGVLLSKRKRKHMKLLSSIEQACDICRLKKLKCSKEKPKCAKC | |
| | LKNNWECRYSPKTKRSPLTRAHLTEVESRLERLEQLFLLIFPREDLDMILKMD | |
| | SLQDIKALLTGLFVQDNVNKDAVTDRLASVETDMPLTLRQHRISATSSSEESS | |
| | NKGQRQLTVSAAAGGSGGSGGSDALDDFDLDMLGSDALDDFDLDMLGSDA | |
| | LDDFDLDMLGSDALDDFDLDMLGS | |
| 229 | MALPVTALLLPLALLLHAARPEQKLISEEDLQVQLVQSGAEVKKPGASVKVS | pIZ343BCMA |
| | CKASGYSFPDYYINWVRQAPGQGLEWMGWIYFASGNSEYNQKFTGRVTMT | 542 |
| | RDTSINTAYMELSSLTSEDTAVYFCASLYDYDWYFDVWGQGTMVTVSSGGG | |
| | GSGGGGSGGGGSDIVMTQTPLSLSVTPGQPASISCKSSQSLVHSNGNTYLHW | |
| | YLQKPGQSPQLLIYKVSNRFSGVPDRFSGSGSGTDFTLKISRVEAEDVGIYYCS | |
| | QSSIYPWTFGQGTKLEIKTTTPAPRPPTPAPTIASQPLSLRPEACFMYVAAAAF | |
| | VLLFFVGCGVLLSRKRRRMKLLSSIEQACDICRLKKLKCSKEKPKCAKCLKN | |
| | NWECRYSPKTKRSPLTRAHLTEVESRLERLEQLFLLIFPREDLDMILKMDSLQ | |
| | DIKALLTGLFVQDNVNKDAVTDRLASVETDMPLTLRQHRISATSSSEESSNKG | |
| | QRQLTVSAAAGGSGGSGGSDALDDFDLDMLGSDALDDFDLDMLGSDALDD | |
| | FDLDMLGSDALDDFDLDMLGS | |
| 230 | MALPVTALLLPLALLLHAARPEQKLISEEDLQVQLVQSGAEVKKPGASVKVS | pIZ361BCMA |
| | CKASGYSFPDYYINWVRQAPGQGLEWMGWIYFASGNSEYNQKFTGRVTMT | 543 |
| | RDTSINTAYMELSSLTSEDTAVYFCASLYDYDWYFDVWGQGTMVTVSSGGG | |
| | GSGGGGSGGGGSDIVMTQTPLSLSVTPGQPASISCKSSQSLVHSNGNTYLHW | |
| | YLQKPGQSPQLLIYKVSNRFSGVPDRFSGSGSGTDFTLKISRVEAEDVGIYYCS | |
| | QSSIYPWTFGQGTKLEIKTTTPAPRPPTPAPTIASQPLSLRPEACFMYVAAAAF | |
| | VLLFFVGCGVLLSKRKRKHMKLLSSIEQACDICRLKKLKCSKEKPKCAKCLK | |
| | NNWECRYSPKTKRSPLTRAHLTEVESRLERLEQLFLLIFPREDLDMILKMDSL | |
| | QDIKALLTGLFVQDNVNKDAVTDRLASVETDMPLTLRQHRISATSSSEESSNK | |
| | GQRQLTVSAAAGGSGGSGGSDALDDFDLDMLGSDALDDFDLDMLGSDALD | |
| | DFDLDMLGSDALDDFDLDMLGS | |
| 231 | MALPVTALLLPLALLLHAARPEQKLISEEDLDIQMTQSPSSLSASVGDRVTITC | pIZ343(4D5-8) |
| | RASQDVNTAVAWYQQKPGKAPKLLIYSASFLYSGVPSRFSGSRSGTDFTLTIS | 542 |
| | SLQPEDFATYYCQQHYTTPPTFGQGTKVEIKRTGSTSGSGKPGSGEGSEVQLV | |
| | ESGGGLVQPGGSLRLSCAASGFNIKDTYIHWVRQAPGKGLEWVARIYPTNGY | |
| | TRYADSVKGRFTISADTSKNTAYLQMNSLRAEDTAVYYCSRWGGDGFYAM | |
| | DVWGQGTLVTVSSGSTTTPAPRPPTPAPTIASQPLSLRPEACFMYVAAAAFVL | |
| | LFFVGCGVLLSRKRRRMKLLSSIEQACDICRLKKLKCSKEKPKCAKCLKNNW | |
| | ECRYSPKTKRSPLTRAHLTEVESRLERLEQLFLLIFPREDLDMILKMDSLQDIK | |
| | ALLTGLFVQDNVNKDAVTDRLASVETDMPLTLRQHRISATSSSEESSNKGQR | |
| | QLTVSAAAGGSGGSGGSDALDDFDLDMLGSDALDDFDLDMLGSDALDDFD | |
| | LDMLGSDALDDFDLDMLGS | |
| 232 | MALPVTALLLPLALLLHAARPEQKLISEEDLDIQMTQSPSSLSASVGDRVTITC | pIZ361(4D5-8) |
| | RASQDVNTAVAWYQQKPGKAPKLLIYSASFLYSGVPSRFSGSRSGTDFTLTIS | 543 |
| | SLQPEDFATYYCQQHYTTPPTFGQGTKVEIKRTGSTSGSGKPGSGEGSEVQLV | |
| | ESGGGLVQPGGSLRLSCAASGFNIKDTYIHWVRQAPGKGLEWVARIYPTNGY | |
| | TRYADSVKGRFTISADTSKNTAYLQMNSLRAEDTAVYYCSRWGGDGFYAM | |
| | DVWGQGTLVTVSSGSTTTPAPRPPTPAPTIASQPLSLRPEACFMYVAAAAFVL | |
| | LFFVGCGVLLSKRKRKHMKLLSSIEQACDICRLKKLKCSKEKPKCAKCLKNN | |
| | WECRYSPKTKRSPLTRAHLTEVESRLERLEQLFLLIFPREDLDMILKMDSLQDI | |
| | KALLTGLFVQDNVNKDAVTDRLASVETDMPLTLRQHRISATSSSEESSNKGQ | |
| | RQLTVSAAAGGSGGSGGSDALDDFDLDMLGSDALDDFDLDMLGSDALDDF | |
| | DLDMLGSDALDDFDLDMLGS | |
| 233 | MALPVTALLLPLALLLHAARPEQKLISEEDLDIQMTQSPSSLSASVGDRVTITC | pIZ343(4D5-7) |
| | RASQDVNTAVAWYQQKPGKAPKLLIYSASFLESGVPSRFSGSRSGTDFTLTIS | 542 |
| | SLQPEDFATYYCQQHYTTPPTFGQGTKVEIKRTGSTSGSGKPGSGEGSEVQLV | |
| | ESGGGLVQPGGSLRLSCAASGFNIKDTYIHWVRQAPGKGLEWVARIYPTNGY | |
| | TRYADSVKGRFTISADTSKNTAYLQMNSLRAEDTAVYYCSRWGGDGFYAM | |
| | DVWGQGTLVTVSSGSTTTPAPRPPTPAPTIASQPLSLRPEACFMYVAAAAFVL | |
| | LFFVGCGVLLSRKRRRMKLLSSIEQACDICRLKKLKCSKEKPKCAKCLKNNW | |
| | ECRYSPKTKRSPLTRAHLTEVESRLERLEQLFLLIFPREDLDMILKMDSLQDIK | |
| | ALLTGLFVQDNVNKDAVTDRLASVETDMPLTLRQHRISATSSSEESSNKGQR | |
| | QLTVSAAAGGSGGSGGSDALDDFDLDMLGSDALDDFDLDMLGSDALDDFD | |
| | LDMLGSDALDDFDLDMLGS | |
| 234 | MALPVTALLLPLALLLHAARPEQKLISEEDLDIQMTQSPSSLSASVGDRVTITC | pIZ361(4D5-7) |
| | RASQDVNTAVAWYQQKPGKAPKLLIYSASFLESGVPSRFSGSRSGTDFTLTIS | 543 |
| | SLQPEDFATYYCQQHYTTPPTFGQGTKVEIKRTGSTSGSGKPGSGEGSEVQLV | |
| | ESGGGLVQPGGSLRLSCAASGFNIKDTYIHWVRQAPGKGLEWVARIYPTNGY | |
| | TRYADSVKGRFTISADTSKNTAYLQMNSLRAEDTAVYYCSRWGGDGFYAM | |
| | DVWGQGTLVTVSSGSTTTPAPRPPTPAPTIASQPLSLRPEACFMYVAAAAFVL | |
| | LFFVGCGVLLSKRKRKHMKLLSSIEQACDICRLKKLKCSKEKPKCAKCLKNN | |
| | WECRYSPKTKRSPLTRAHLTEVESRLERLEQLFLLIFPREDLDMILKMDSLQDI | |
| | KALLTGLFVQDNVNKDAVTDRLASVETDMPLTLRQHRISATSSSEESSNKGQ | |
| | RQLTVSAAAGGSGGSGGSDALDDFDLDMLGSDALDDFDLDMLGSDALDDF | |
| | DLDMLGSDALDDFDLDMLGS | |
| 235 | MALPVTALLLPLALLLHAARPDYKDDDDKEVQLLESGGGLVQPGGSLRLSC | pRay068A |
| | AASGFTFSSYAMSWVRQAPGKGLEWVSSISGSGDYIYYADSVKGRFTISRDIS | 416 |
| | KNTLYLQMNSLRAEDTAVYYCAKEGTGANSSLADYRGQGTLVTVSSTTTPA | |
| | PRPPTPAPTIASQPLSLRPEACFMYVAAAAFVLLFFVGCGVLLSKRKRKHMKL | |
| | LSSIEQACDICRLKKLKCSKEKPKCAKCLKNNWECRYSPKTKRSPLTRAHLTE | |
| | VESRLERLEQLFLLIFPREDLDMILKMDSLQDIKALLTGLFVQDNVNKDAVTD | |
| | RLASVETDMPLTLRQHRISATSSSEESSNKGQRQLTVSAAAGGSGGSGGSDA | |
| | LDDFDLDMLGSDALDDFDLDMLGSDALDDFDLDMLGSDALDDFDLDMLGS | |
| 236 | MALPVTALLLPLALLLHAARPDYKDDDDKEVQLLESGGGLVQPGGSLRLSC | pRay068B |
| | AASGFTFSSYAMSWVRQAPGKGLEWVSSISGSGDYIYYADSVKGRFTISRDIS | 426 |
| | KNTLYLQMNSLRAEDTAVYYCAKEGTGANSSLADYRGQGTLVTVSSFVPVF | |
| | LPAKPTTTPAPRPPTPAPTIASQPLSLRPEACFMYVAAAAFVLLFFVGCGVLLS | |
| | KRKRKHMKLLSSIEQACDICRLKKLKCSKEKPKCAKCLKNNWECRYSPKTK | |
| | RSPLTRAHLTEVESRLERLEQLFLLIFPREDLDMILKMDSLQDIKALLTGLFVQ | |
| | DNVNKDAVTDRLASVETDMPLTLRQHRISATSSSEESSNKGQRQLTVSAAAG | |
| | GSGGSGGSDALDDFDLDMLGSDALDDFDLDMLGSDALDDFDLDMLGSDAL | |
| | DDFDLDMLGS | |
| 237 | MALPVTALLLPLALLLHAARPEQKLISEEDLDIQMTQTTSSLSASLGDRVTISC | pIZ370 |
| | RASQDISKYLNWYQQKPDGTVKLLIYHTSRLHSGVPSRFSGSGSGTDYSLTIS | 543 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSTTTPAPRPPTPAPTIASQPLSLRPEACATVVIVVCVSFLVFMII | |
| | LGVFRIRAAHRRTMRMKLLSSIEQACDICRLKKLKCSKEKPKCAKCLKNNWE | |
| | CRYSPKTKRSPLTRAHLTEVESRLERLEQLFLLIFPREDLDMILKMDSLQDIKA | |
| | LLTGLFVQDNVNKDAVTDRLASVETDMPLTLRQHRISATSSSEESSNKGQRQ | |
| | LTVSAAAGGSGGSGGSDALDDFDLDMLGSDALDDFDLDMLGSDALDDFDL | |
| | DMLGSDALDDFDLDMLGS | |
| 238 | MALPVTALLLPLALLLHAARPEQKLISEEDLDIQMTQTTSSLSASLGDRVTISC | pIZ371 |
| | RASQDISKYLNWYQQKPDGTVKLLIYHTSRLHSGVPSRFSGSGSGTDYSLTIS | 541 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSTTTPAPRPPTPAPTIASQPLSLRPEACIATVVIIISVCMLVFVV | |
| | AMGVYRVRIAHQHMKLLSSIEQACDICRLKKLKCSKEKPKCAKCLKNNWEC | |
| | RYSPKTKRSPLTRAHLTEVESRLERLEQLFLLIFPREDLDMILKMDSLQDIKAL | |
| | LTGLFVQDNVNKDAVTDRLASVETDMPLTLRQHRISATSSSEESSNKGQRQL | |
| | TVSAAAGGSGGSGGSDALDDFDLDMLGSDALDDFDLDMLGSDALDDFDLD | |
| | MLGSDALDDFDLDMLGS | |
| 239 | MALPVTALLLPLALLLHAARPEQKLISEEDLDIQMTQTTSSLSASLGDRVTISC | pTMD201 |
| | RASQDISKYLNWYQQKPDGTVKLLIYHTSRLHSGVPSRFSGSGSGTDYSLTIS | 537 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSTTTPAPRPPTPAPTIASQPLSLRPEACATVVIVVCVSFLVFMII | |
| | LGVFRKRRRMKLLSSIEQACDICRLKKLKCSKEKPKCAKCLKNNWECRYSPK | |
| | TKRSPLTRAHLTEVESRLERLEQLFLLIFPREDLDMILKMDSLQDIKALLTGLF | |
| | VQDNVNKDAVTDRLASVETDMPLTLRQHRISATSSSEESSNKGQRQLTVSAA | |
| | AGGSGGSGGSDALDDFDLDMLGSDALDDFDLDMLGSDALDDFDLDMLGSD | |
| | ALDDFDLDMLGS | |
| 240 | MALPVTALLLPLALLLHAARPEQKLISEEDLDIQMTQTTSSLSASLGDRVTISC | pTMD202 |
| | RASQDISKYLNWYQQKPDGTVKLLIYHTSRLHSGVPSRFSGSGSGTDYSLTIS | 538 |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGGSYAMDY | |
| | WGQGTSVTVSSTTTPAPRPPTPAPTIASQPLSLRPEACIATVVIIISVCMLVFVV | |
| | AMGVYRKRRRMKLLSSIEQACDICRLKKLKCSKEKPKCAKCLKNNWECRYS | |
| | PKTKRSPLTRAHLTEVESRLERLEQLFLLIFPREDLDMILKMDSLQDIKALLTG | |
| | LFVQDNVNKDAVTDRLASVETDMPLTLRQHRISATSSSEESSNKGQRQLTVS | |
| | AAAGGSGGSGGSDALDDFDLDMLGSDALDDFDLDMLGSDALDDFDLDMLG | |
| | SDALDDFDLDMLGS | |
| 241 | ATVVIVVCVSFLVFMIILGVF | TMD Domain |
| | | of SEQ ID |
| | | NOS: 237 and |
| | | 239 |
| 242 | IATVVIIISVCMLVFVVAMGVY | TMD Domain |
| | | of SEQ ID |
| | | NOS: 238 and |
| | | 240 |

### REFERENCES

D.L. Porter et al., N Engl J Med (2011) 365(8): 725-33.
L. Morsut et al., Cell (2016) 164(4): 780-91.
M.F. Naso et al., BioDrugs (2017) 31(4):317-34.
M. Nasri et al., Cytotechnology (2014) 66(6):1031-38.
K.T. Roybal et al., Cell (2016) 167(2):419-32.
Samulski et al., Annu Rev Virol (2014) 1:427.
Sakuma et al., Biochem J (2012) 443:603.
D.J. Watson et al., “Viral vectors for gene therapy: methods and protocols” 2003 (Totowa, NJ, USA: Humana Press); pp. 383-404.

What is claimed is:

1. A chimeric polypeptide comprising, from N-terminus to C-terminus:
a) an extracellular ligand-binding domain having a binding affinity for a selected ligand;
b) a linking polypeptide having:
(i) at least about 80% sequence identity to a Notch juxtamembrane domain (JMD);
(ii) at least about 80% sequence identity to a Notch JMD wherein the LIN-12-Notch repeat (LNR) and/or a heterodimerization domain (HD) of a Notch receptor has been deleted;
(iii) at least about 80% sequence identity to a polypeptide hinge domain;
(iv) at least about 80% sequence identity to a Robol JMD including at least one fibronectin repeat; or
(v) a polypeptide having about 2 to about 40 amino acids;
c) a transmembrane domain (TMD) comprising one or more ligand-inducible proteolytic cleavage sites;
d) a stop-transfer sequence (STS), wherein the STS is heterologous to the TMD;
and
e) an intracellular domain comprising a transcriptional regulator, wherein binding of the selected ligand to the extracellular ligand-binding domain induces cleavage at the ligand-inducible proteolytic cleavage site between the transcriptional regulator and the linking polypeptide.

2. The chimeric polypeptide of claim 1, wherein the STS comprises an amino acid sequence having at least about 80% sequence identity to any one of SEQ ID NOs: 100-174 and 207.

3. The chimeric polypeptide of either claim 1 or 2, wherein the STS comprises an amino acid sequence having at least about 90% sequence identity to any one of SEQ ID NOs:
100-174 and 207.

4. The chimeric polypeptide of any one of claims 1 to 3, wherein the STS comprises an amino acid sequence having at least about 95% sequence identity to any one of SEQ ID NOs: 100-174 and 207.

5. The chimeric polypeptide of any one of claims 1 to 4, wherein the STS comprises an amino acid sequence that differs by no more than one amino acid from any one of SEQ ID NOs: 100-102, 104-113, 115-116, 118-174, and 207.

6. The chimeric polypeptide of any one of claims 1 to 5, wherein the STS comprises an amino acid sequence having at least about 80% sequence identity to any one of SEQ ID NOs: 105, 107, 123, 143, 146, 153-155, 161, 163, 164, 166, 171, 172, 174, and 207.

7. The chimeric polypeptide of any one of claims 1 to 6, wherein the STS comprises an amino acid sequence having at least about 90% sequence identity to any one of SEQ ID NOs: 105, 107, 123, 143, 146, 153-155, 161, 163, 164, 166, 171, 172, 174, and 207.

8. The chimeric polypeptide of any one of claims 1 to 7, wherein the STS comprises an amino acid sequence having at least about 95% sequence identity to any one of SEQ ID NOs: 105, 107, 123, 143, 146, 153-155, 161, 163, 164, 166, 171, 172, 174, and 207.

9. The chimeric polypeptide of any one of claims 1 to 8, wherein the STS comprises an amino acid sequence that differs by no more than one amino acid from any one of SEQ ID NOs: 105, 107, 123, 143, 146, 153-155, 161, 163, 164, 166, 171, 172, 174, and 207.

10. The chimeric polypeptide of any one of claims 1 to 9, wherein the STS is selected from the group of STS sequences consisting of APLP1, APLP2, APP, TGBR3, CSF1R, CXCL16, CX3CL1, DAG1, DCC, DNER, DSG2, CDH1, GHR, HLA-A, IFNAR2, IGF1R, IL1R1, ERN2, KCNE1, KCNE2, CHL1, LRP1, LRP2, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDHS, PKHD1, NECTIN1, KL, IL6R, EFNB1, CD44, CLSTN1, LRP8, PCDHGC3, NRG1, LRP1B, JAG2, EFNB2, DLL1, CLSTN2, EPCAM, ErbB4, KCNE3, CDH2, NRG2, PTPRK, BTC, EPHA4, IL1R2, KCNE4, SCN2B, Nradd, PTPRM, Notch1, Notch2, Notch3, and Notch4.

11. The chimeric polypeptide of any one of claims 1 to 10, wherein the extracellular domain comprises an antigen-binding moiety capable of binding to a ligand on the surface of a cell.

12. The chimeric polypeptide any one of claims 1 to 11, wherein the cell is a pathogen.

13. The chimeric polypeptide of any one of claims 1 to 12, wherein the ligand comprises a protein or a carbohydrate.

14. The chimeric polypeptide of any one of claims 1 to 13, wherein the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE, alkaline phosphatase, placental-like 2 (ALPPL2), B-cell maturation antigen (BCMA), green fluorescent protein (GFP), enhanced green fluorescent protein (eGFP), and signal regulatory protein α (SIRPα).

15. The chimeric polypeptide of any one of claims 1 to 14, wherein the ligand is selected from cell surface receptors, adhesion proteins, integrins, mucins, lectins, tumor associated antigens, and tumor-specific antigens.

16. The chimeric polypeptide of any one of claims 1 to 15, wherein the ligand is a tumor-associated antigen or a tumor-specific antigen.

17. The chimeric polypeptide of any one of claims 1 to 16, wherein the extracellular ligand-binding domain comprises the ligand-binding portion of a receptor.

18. The chimeric polypeptide of any one of claims 1 to 17, wherein the antigen-binding moiety is selected from the group consisting of an antibody, a nanobody, a diabody, a triabody, or a minibody, a F(ab′)2 fragment, a Fab fragment, a single chain variable fragment (scFv), a single domain antibody (sdAb), or a functional fragment of an antibody.

19. The chimeric polypeptide of claim 18, wherein the antigen-binding moiety comprises an scFv.

20. The chimeric polypeptide of any one of claims 11 to 19, wherein the antigen-binding moiety is a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD117), MUC1, NCAM, PAP, PDGFR-β, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

21. The chimeric polypeptide of claim 20, wherein the tumor-associated antigen is CD19, CEA, HER2, MUC1, CD20, ALPPL2, or EGFR.

22. The chimeric polypeptide of claim 21, wherein the tumor-associated antigen is CD19.

23. The chimeric polypeptide of any one of claims 1 to 22, wherein the ligand-inducible proteolytic cleavage site is a y secretase cleavage site.

24. The chimeric polypeptide of any one of claims 1 to 23, wherein the transcriptional regulator comprises a transcriptional activator, or a transcriptional repressor.

25. The chimeric polypeptide of any one of claims 1 to 24, wherein the intracellular domain comprises a nuclear localization sequence and a transcriptional regulator sequence selected from Gal4-VP16, Gal4-VP64, tetR-VP64, ZFHD1-VP64, Gal4-KRAB, and HAP1-VP16.

26. The chimeric polypeptide of any one of claims 1 to 25, further comprising a signal sequence, a detectable label, a tumor-specific cleavage site, a disease-specific cleavage site, or a combination thereof.

27. The chimeric polypeptide of any one of claims 1 to 19, wherein the linking polypeptide comprises an amino acid sequence having at least 80% sequence identity to any one of SEQ ID NOS: 178-180 and 183-198.

28. The chimeric polypeptide of claim 27, wherein the linking polypeptide comprises an amino acid sequence having at least 90% sequence identity to any one of SEQ ID NOS: 178-180 and 183-198.

29. The chimeric polypeptide of claim 29, wherein the linking polypeptide comprises an amino acid sequence having at least 95% sequence identity to any one of SEQ ID NOS: 178-180 and 183-198.

30. The chimeric polypeptide of claim 29, wherein the linking polypeptide comprises an amino acid sequence substantially identical to any one of SEQ ID NOS: 178-180 and 183-198.

31. The chimeric polypeptide of claim 30, wherein the TMD comprises an amino acid sequence substantially identical to either of SEQ ID NOS: 181, 206, 241, and 242.

32. The chimeric polypeptide of claim 32, wherein the TMD comprises an amino acid sequence having at least 90% sequence identity to either of SEQ ID NOS: 181, 206, 241, and 242.

33. The chimeric polypeptide of any one of claims 1 to 33, wherein the TMD comprises an amino acid sequence having at least 95% sequence identity to either of SEQ ID NOS: 181, 206, 241, and 242.

34. The chimeric polypeptide of any one of claims 1 to 33, wherein the TMD comprises an amino acid sequence substantially identical to either of SEQ ID NOS: 181, 206, 241, and 242.

35. The chimeric polypeptide of any one of claims 1 to 34, wherein:
a) the linking polypeptide comprises an amino acid sequence having at least 80% sequence identity to any one of SEQ ID NOS: 178-180 and 183-198;
b) the TMD comprises an amino acid sequence having at least 80% sequence identity to any one of SEQ ID NO: 181, 206, 241, and 242; and
c) the stop transfer sequence domain comprises an amino acid sequence having at least 80% sequence identity to SEQ ID NO: 100-174 and 207.

36. The chimeric polypeptide of any one of claims 1 to 35, wherein the chimeric polypeptide comprises an amino acid sequence having at least 80% sequence identity to any one of SEQ ID NOS: 1-99.

37. A recombinant nucleic acid comprising a nucleotide sequence encoding a chimeric polypeptide according to any one of claims 1 to 29.

38. The recombinant nucleic acid of claim 30, wherein the nucleotide sequence is incorporated into an expression cassette or an expression vector.

39. The recombinant nucleic acid of claim 31, wherein the expression vector is a viral vector.

40. The recombinant nucleic acid of claim 32, wherein the viral vector is a lentiviral vector, an adenovirus vector, an adeno-associated virus vector, or a retroviral vector.

41. A recombinant cell comprising:
a) a chimeric polypeptide according to any one of claims 1 to 29; and/or
b) a recombinant nucleic acid according to any one of claims 30 to 33.

42. The recombinant cell of claim 41, wherein the cell is a mammalian cell.

43. The recombinant cell of claim 35, wherein the mammalian cell is an immune cell, a neuron, an epithelial cell, and endothelial cell, or a stem cell.

44. The recombinant cell of claim 36, wherein the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

45. The recombinant cell of any one of claims 34 to 37, further comprising:
a) a second chimeric polypeptide according to any one of claims 1 to 29; and/or
b) a second nucleic acid according to any one of claims 30 to 33;
wherein the first chimeric polypeptide and the second chimeric polypeptide do not have the same sequence, and/or the first nucleic acid or the second nucleic acid do not have the same sequence.

46. The recombinant cell of claim 38, wherein the first chimeric polypeptide modulates the expression and/or activity of the second chimeric polypeptide.

47. The recombinant cell of any one of claims 34 to 39, further comprising:
a) an expression cassette encoding a protein of interest operably linked to a promoter, wherein expression of the protein is modulated by the chimeric receptor transcriptional regulator.

48. The recombinant cell of claim 40, wherein the protein of interest is heterologous to the cell.

49. The recombinant cell of claim 40 or 41, wherein the promoter is GAL4.

50. The recombinant cell of claim 39 or 49, wherein the protein of interest is a cytokine, a cytotoxin, a chemokine, an immunomodulator, a pro-apoptotic factor, an anti-apoptotic factor, a hormone, a differentiation factor, a dedifferentiation factor, an immune cell receptor, or a reporter.

51. A cell culture comprising a recombinant cell according to any one of claims 41 to 50, and a culture medium.

52. A pharmaceutical composition comprising a pharmaceutically acceptable carrier, and one or more of the following:
a) a recombinant nucleic acid according to any one of claims 37 to 40; or
b) a recombinant cell according to any one of claims 41 to 50.

53. The pharmaceutical composition of claim 52, wherein the composition comprises a recombinant nucleic acid according to any one of claims 37 to 40, and a pharmaceutically acceptable carrier.

54. The pharmaceutical composition of claim 53, wherein the recombinant nucleic acid is encapsulated in a viral capsid or a lipid nanoparticle.

55. A method for modulating an activity of a cell, the method comprising:
a) providing a recombinant cell according to any one of claims 41 to 50; and
b) contacting the recombinant cell with the selected ligand, wherein binding of the selected ligand to the extracellular ligand-binding domain induces cleavage of a ligand-inducible proteolytic cleavage site and releases the transcriptional regulator, wherein the released transcriptional regulator modulates an activity of the recombinant cell.

56. The method of claim 55, the contacting is carried out in vivo, ex vivo, or in vitro.

57. The method of any one of claims 55 to 56, wherein the activity of the cell is selected from the group consisting of: expression of a selected gene of the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell.

58. The method of any one of claims 55 to 57, wherein the released transcriptional regulator modulates expression of a gene product of the cell.

59. The method of any one of claims 55 to 58, wherein the released transcriptional regulator modulates expression of a heterologous gene product.

60. The method of any one of claims 55 to 59, wherein the gene product of the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor, a toxin, a toxin derived protein, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

61. The method of any one of claims 55 to 60, wherein the released transcriptional regulator modulates differentiation of the cell, and wherein the cell is an immune cell, a stem cell, a progenitor cell, or a precursor cell.

62. A method for inhibiting a target cell in an individual, the method comprising administering to the individual an effective number of the recombinant cell according to any one of claims 41 to 50, wherein the recombinant cell inhibits the target cell in the individual.

63. The method of claim 62, wherein the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T cell lymphoma cell, a renal cancer cell, a sarcoma cell, a stomach cancer cell, a carcinoma cell, a mesothelioma cell, or a sarcoma cell.

64. The method of claim 62, wherein the target cell is a pathogenic cell.

65. A method for the treatment of a health condition in an individual in need thereof, the method comprising: administering to the individual a first therapy comprising an effective number of the recombinant cell according to any one of claims 41 to 50, wherein the recombinant cell treats the health condition in the individual.

66. The method of claim 65, further comprising administering to the individual a second therapy.

67. The method of claim 66, wherein the second therapy is selected from the group consisting of chemotherapy, radiotherapy, immunotherapy, hormonal therapy, or toxin therapy.

68. The method of any one of claims 65 to 67, wherein the first therapy and the second therapy are administered together, in the same composition or in separate compositions.

69. The method claim 68, wherein the first therapy and the second therapy are administered at the same time.

70. The method of any one of claims 66 to 67, wherein the first therapy and the second therapy are administered sequentially.

71. The method of claim 70, wherein the first therapy is administered before the second therapy.

72. The method of claim 70, wherein the first therapy is administered after the second therapy.

73. The method of claim 70, wherein the first therapy and the second therapy are administered in rotation.

74. A system for modulating an activity of a cell, inhibiting a target cancer cell, or treating a health condition in an individual in need thereof, wherein the system comprises one or more of the following:
a) a chimeric polypeptide according to any one of claims 1 to 36;
b) a recombinant nucleic acid according to any one of claims 37 to 40;
c) a recombinant cell according to any one of claims 41 to 50; and
d) a pharmaceutical composition according to any one of claims 52 to 54.

75. A method for making the recombinant cell according to any one of claims 41 to 50, comprising:
a) providing a cell capable of protein expression; and
b) contacting the provided cell with a recombinant nucleic acid according to any one of claims 37 to 40.

76. The method of claim 75, wherein the cell is obtained by leukapheresis performed on a sample obtained from a human subject, and the cell is contacted ex vivo.

77. The method of claim 75, wherein the recombinant nucleic acid is encapsulated in a viral capsid or a lipid nanoparticle.

78. The use of one or more of the following for the treatment of a health condition:
a) a chimeric polypeptide according to any one of claims 1 to 36;
b) a recombinant nucleic acid according to any one of claims 37 to 40;
c) a recombinant cell according to any one of claims 41 to 50; and
d) a composition according to any one of claims 52 to 54.

79. The use of claim 78, wherein the health condition is cancer.

80. The use of the invention of any one of claims 1 to 79, for the manufacture of a medicament for the treatment of a health condition.
